(19) United States
(12) Patent Application Publication
(10) Pub. No.: US 2023/0279055 A1
(43) Pub. Date: Sep. 7, 2023

(54) De Novo Design of Immunoglobulin-like Domains

(71) Applicants: UNIVERSITY OF WASHINGTON, Seattle, WA (US); El Consejo Superior de Investigaciones Científicas, Madrid (ES)

(72) Inventors: David Baker, Seattle, WA (US); Tamuka Chidyausiku, Seattle, WA (US); Jason C. Klima, Seattle, WA (US); Enrique Marcos Benteo, Seattle, WA (US); F. Xavier Gomis Rüth, Madrid (ES); Soraia Dos Reis Mendes, Madrid (ES); Ulrich Eckhard, Madrid (ES); Marta Nadal Rovira, Madrid (ES); Jorge Luis Roel Touris, Madrid (US)

(21) Appl. No.: 18/177,367

(22) Filed: Mar. 2, 2023

Related U.S. Application Data
(60) Provisional application No. 63/316,733, filed on Mar. 4, 2022.

(51) Int. Cl. C07K 14/00; C12N 15/63

(52) U.S. Cl. CPC C07K 14/00; C12N 15/63

(57) ABSTRACT

The disclosure provides antibody-like polypeptides of the formula X1-X2-X3-X4-X5-X6-X7-X8-X9-X10-X11-X12-X13-X14-X15, wherein the domains are as defined herein, nucleic acids encoding the polypeptides, and methods for use and design of the polypeptides.

### CROSS REFERENCE

This application claims priority to U.S. Provisional Patent Application Ser. No. 63/316,733 filed Mar. 4, 2022, incorporated by reference herein in its entirety.

### FEDERAL FUNDING STATEMENT

This invention was made with government support under Grant No. FA8750-17-C-0219, awarded by the Defense Advanced Research Projects Agency. The government has certain rights in the invention

### REFERENCE TO AN ELECTRONIC SEQUENCE LISTING

A computer readable form of the Sequence Listing is filed with this application by electronic submission and is hereby incorporated by reference in its entirety. The Sequence Listing is contained in the XML file created on Feb. 27, 2023, having the name “22-0095_SequenceListing.xml” and is 379,834 bytes in size.

### BACKGROUND

Antibodies and antibody derivatives such as nanobodies contain immunoglobulin-like (Ig) β-sandwich scaffolds which anchor the hypervariable antigen-binding loops and constitute the largest growing class of drugs. Current engineering strategies for this class of compounds rely on naturally existing Ig frameworks, which can be hard to modify and have limitations in manufacturability, designability and range of action.

### SUMMARY

In one aspect, the disclosure provides polypeptides comprising the formula X1-X2-X3-X4-X5-X6-X7-X8-X9-X10-X11-X12-X13-X14-X15, wherein:
X1 is optional, and when present comprises 1 2, or 3 residues with loop secondary structure;
X2 comprises 5, 6, 7, or 8 residues with β-strand secondary structure;
X3 comprises 2, 3 or 4 residues with loop secondary structure, forming a β-hairpin tertiary structure motif;
X4 comprises 6, 7, or 8 residues with β-strand secondary structure;
X5 comprises 3, 4, 5 or 6 residues with loop secondary structure, forming a β-arch tertiary structure motif (i.e., a connection between X4 and X6);
X6 comprises 6 or 7 residues with β-strand secondary structure;
X7 comprises 2, 3, or 4 residues with loop secondary structure, forming a β-hairpin tertiary structure motif;
X8 comprises 6 or 7 residues with β-strand secondary structure;
X9 comprises 3, 4 or 5 residues with loop secondary structure, forming a β-arch tertiary structure motif (i.e., a connection between X8 and X10);
X10 comprises 4, 5, 6, 7 or 8 residues with β-strand secondary structure;
X11 comprises one of the following, forming a β-arch tertiary structure motif:
   3, 4, 5, 6, 7, or 8 residues with loop secondary structure; or
   2, 3, or 4 residues with loop secondary structure, followed by 3, 4, 5, or 6 residues with α-helical secondary structure, followed by 1, 2, or 3 residues with loop secondary structure;
X12 comprises 6, 7, or 8 residues with β-strand secondary structure;
X13 comprises 2, 3, or 4 residues with loop secondary structure, forming a β-hairpin tertiary structure motif;
X14 comprises 5, 6, 7, or 8 residues with β-strand secondary structure; and
X15 is optional, and when present comprises 1, 2 or 3 residues with loop secondary structure.

In one embodiment, neither X1 nor X15 are present; one of X1 or X15 is present (for example, X1 is present; or X15 is present); or X1 and X15 are both present. In another embodiment, X11 comprises 2, 3, or 4 residues with loop secondary structure, followed by 3, 4, 5, or 6 residues with α-helical secondary structure, followed by 1, 2, or 3 residues with loop secondary structure. In a further embodiment, X11 comprises a domain structure of 2L5H1L, 2L5H2L, 2L6H1L, 3L4H2L, 3L5H2L, 4L4H2L, 4L4H3L (where “L” stands for loop secondary structure and “H” stands for α-helical secondary structure). In one embodiment, 1, 2, or all 3 β-arch motifs (X5, X9, and X11) have atoms involved in hydrogen bonds between (i) two backbone atoms, (ii) one backbone and a sidechain atom, or (iii) two-sidechain atoms.

In another embodiment, 1, 2, 3, 4, 5, 6, 7, or all 8 of the following are true:
(a) X2 forms an antiparallel β-strand pairing with X4;
(b) X4 forms an antiparallel β-strand pairing with X10;
(c) X2, X4, and X10 form a first layer of β-sheets, with X2 and X10 as edge β-strands;
(d) X6 forms an antiparallel β-strand pairing with X8;
(e) X6 forms an antiparallel β-strand pairing with X12;
(f) X12 forms an antiparallel β-strand pairing with X14;
(g) X6, X8, X12 and X14 form a second layer of β-sheets, with X8 and X14 as edge β-strands; and/or
(h) the first layer of β-sheets and the second layer of β-sheets form a β-sandwich tertiary structure motif.

In a further embodiment, X4, X6, and X12 comprise alternating hydrophobic and hydrophilic residues, and optionally wherein 1, 2, 3, or all 4 of X2, X8, X10, and X14 comprise alternating hydrophobic and hydrophilic residues.

In one embodiment, X4, X6, and X12 independently comprise the amino acid sequence selected from the group consisting of SEQ ID NO:1-87. In another embodiment, 1, 2, 3, or all 4 of X2, X8, X10, and X14 comprise alternating hydrophobic and hydrophilic residues, independently comprising the amino acid sequence selected from the following group consisting of SEQ ID NO:88-123.

In one embodiment, X2, X8, X10, and X14 comprise at least one polar amino acid residue selected from Arg, Lys, Glu, Gln, and His. In another embodiment, X2, X8, X10, and X14 independently comprise the amino acid sequence selected from the group consisting of SEQ ID NO:88-203. In a further embodiment, X2, X4, X6, X8, X10, X12, and X14 independently comprise an amino acid sequence selected from the group consisting of SEQ ID NO:1-203.

In one embodiment, X5, X9, and X11 comprise (i) at least one polar amino acid selected from Asn, Ser, Thr, Glu, and Gln in the domain or in the residue immediately preceding or following the domain, where the polar residue is involved in at least one hydrogen bond between (i) two backbone atoms, (ii) one backbone and a sidechain atom, or (iii) two-sidechain atoms, and (iv) a glycine or proline residue. In another embodiment, X5, X9, and X11 independently comprise the amino acid sequence selected from the group consisting of SEQ ID NO:236-286, and DGP, DRP, EGP, NGP, NKG, NPG, PPG, and RGE.

In a further embodiment, the X3, X7, and X13 domains each comprise at least one glycine residue. In one embodiment, the X3, X7, and X13 domains independently comprise the amino acid sequence selected from the group consisting of APGT (SEQ ID NO:287), DG, EG, GD, GE, GG, GK, GKGV (SEQ ID NO:288), GN, KGNR (SEQ ID NO:289), KNN, NG, PG, and RGDS (SEQ ID NO:290).

In one embodiment, at least two non-contiguous β-strands include a cysteine residue, wherein the at least two non-contiguous β-strand cysteine residues are capable of forming a disulfide bond. In another embodiment, the first residue of the X6 domain and the last residue of the X12 domain are cysteine residues capable of forming a disulfide bond. In a further embodiment, the polypeptide comprises a disulfide bond between non-contiguous β-strands.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical, not including any functional domain insertions, to the amino acid sequence selected from group consisting of SEQ ID NO:204-235 and 291-301.

In another embodiment, the polypeptide further comprises one or more functional domains inserted into the polypeptide. In one embodiment, one or more functional domains are inserted into domain X1 and/or X15. In a further embodiment, one or more functional domains are inserted into domain X3, X5, X7, X9, X11, and/or X13.

In another embodiment, any one or more domain further comprises an attached fluorophore, chemiluminescent compound, or reactive moieties for “click” chemistry.

In one embodiment, the disclosure provides multimers, comprising 2, 3, 4, 5, 6, or more copies of the polypeptide of any embodiment or combination of embodiments herein. In another embodiment, the multimer comprises a dimer. In another embodiment, one or more polypeptides in the multimer are deleted for domains X1, X14, and/or X15; optionally wherein one or more polypeptides in the multimer are deleted for domains X14 and X15.

In other embodiments, the disclosure provides nucleic acids encoding the polypeptide or multimer of any embodiment or combination of embodiments herein; an expression vector comprising a nucleic acid of the disclosure operatively linked to a suitable control sequence; host cells comprising a polypeptide, multimer, nucleic acid, or expression vector of any embodiment or combination of embodiments herein; pharmaceutical composition comprising a polypeptide, multimer, nucleic acid, expression vector, or host cell of any embodiment or combination of embodiments herein; and a pharmaceutically acceptable carrier; methods for use of a polypeptide, multimer, nucleic acid, expression vector, host cell, or pharmaceutical composition of any embodiment or combination of embodiments herein; and methods for designing the polypeptide or multimer of any preceding claim, comprising any method as disclosed herein.

### DESCRIPTION OF THE FIGURES

FIG. 1. Design rules for cross-β motifs in β-sandwiches. a, Cartoon representation of a 7-stranded immunoglobulin-like domain model formed by two β-sheets packing face-to-face, and the corresponding cross-β motif, which generates rotations and translations between the two opposing β-sheets. b, Topology diagram of a cross-β motif with circles and arrows representing β-strand residue positions and connections, respectively. Dark- and light-colored circles correspond to residues with side chains pointing inwards or protruding from the sandwich, respectively. c, Efficiency of pairs of common β-arch loop geometries (described with ABEGO backbone torsions) in forming cross-β motifs obtained from Rosetta™ folding simulations. Loop geometries were classified in four groups according to the sidechain directions of the adjacent residues. Black boxes highlight loop combinations observed in natural Ig domains. On the right, changes in cross-β motif geometry due to loop geometry. d, β-arch helices are formed by a short α-helix connected to the adjacent β-strands with short loops, and are complementary to β-arch loops for connecting cross-β motifs. e, Topology diagram of a 7-stranded Ig domain. β-arch loops are indicated as Lᵢ, where i is the β-arch number. f, Examples of de novo Ig backbones generated with different geometries and β-arch connections following the described rules.

FIG. 2. Folding and stability of designed proteins. a, Examples of design models. b, Simulated folding energy landscapes, with each dot representing the lowest energy structure obtained from ab initio folding trajectories starting from an extended chain or local relaxation of the designed structure. The x-axis depicts the Cα-RMSD from the designed model and the y-axis, the Rosetta™ all-atom energy. c, Far-ultraviolet circular dichroism spectra (25° C., 55° C., 75° C., 95° C.).

FIG. 3. Structural characterization of dIG14. a, Cartoon representation of the dIG14 design model in comparison with the five AlphaFold™ predicted models. The RMSD with respect to the first AlphaFold™ model is shown. b, dIG14 design model in comparison with the crystal structure. Sidechain packing interactions in the non-terminal edge β-strands were well recapitulated in the crystal structure (left inset). Change in the strand-pairing register observed in the crystal structure as highlighted by the two arrows (right inset). c, Homodimer interface by antiparallel pairing between β-strands 1 and 6 enabled by flipping out of the C-terminal β-strand; the monomer core becomes more accessible and the interface is primarily formed by hydrophobic contacts (right inset).

FIG. 4. Crystal structure of dIG8-CC and functional loop scaffolding. a, dIG8-CC design model in comparison with the crystal structure. b, cross-β motif connections and core sidechain interactions in the design and the crystal structure. The β-arch helix and loop conformations are well preserved across monomer copies in the crystal asymmetric units (insets). c, Homodimer interface by parallel pairing between the two terminal β-strands, which are stabilized through hydrophobic and salt-bridge interactions (inset). d, Computational model of dIG8-CC with a grafted EF-hand motif (design EF61_dIG8-CC, cartoon), showing Th³⁺ (sphere) bound to EF-hand motif residues (sticks). Th³⁺ luminescence is sensitized by absorption of light by a proximal tyrosine residue on the EF-hand motif with subsequent fluorescence resonance energy transfer (FRET) to Tb³⁺, resulting in Tb³⁺ luminescence. e, Means (lines) and s.d. of the means (shadings) of three technical replicates showing luminescence emission spectra in 10 mM Th³⁺ final concentrations for EF61_dIG8-CC and dIG8-CC at 500 μg/mL, with phosphate-buffered saline control without protein. f, Background-subtracted Th³⁺ luminescence from excitation at 280 nm and emission at 544 nm for EF61_dIG8-CC and dIG8-CC normalized to dIG8-CC at 500 μg/mL final concentrations. Error bars represent the s.d. of the mean of three technical replicates.

FIG. 5. Topology of immunoglobulin-like domains. a, Cartoon three-dimensional representation of an Ig structure (left), and strand pairing organization of the constituent seven β-strands (right). Cross-β interactions have higher sequence separation (high contact order) than β-hairpins, which slows down folding. b, From the folding and design perspective, the main limiting factor for correctly assembling the Ig structure is formation of the cross-β motif, since the three β-hairpins can easily form independently of each other.

FIG. 6. Frequently observed p-arch loops in naturally occurring protein structures. a, The Ramachandran plot is conveniently discretized in ABEGO torsion bins describing local backbone geometry at the residue level (“A”: right-handed α-helix region; “B”, extended region; “G”, left-handed α-helix region; “E”, extended region with positive ϕ; and “O”, if the peptide bond deviates from planarity). b, Definition of the β-arch sidechain orientation based on the relative orientation between the translation vector (v₁) and the C_{α}C_{β} vector of the two adjacent β-strand residues. If the C_{α}C_{β} vector of the preceding residue is oriented in the same direction as v₁ the sidechain orientation is “⬇”, otherwise “⬆”. The same applies to the residue following the loop but considering −v₁ as the translation vector. Loop positions are colored according to their ABEGO bin, as shown in panel a. c, β-arch loops (ranging between 3 and 5 residues) spanning the four possible sidechain orientations that are most frequently observed in a non-redundant set of naturally occurring protein structures.

FIG. 7 Coupling between the two β-arches forming cross-β motifs. a, β-arch sliding distance definition. Cartoon representation (left) and diagram (right) of a cross-β motif. We define v₁ and v₂ as the translation vectors connecting the Cα's of the residues preceding and following β-arch loops 1 and 2, respectively; and the S₃₁ vector between the centers of the two N-terminal strands (1 and 3). The sliding distance is the projection of the β-arch translation vectors onto the S₃₁ vector. b, Correlation between the two β-arch sliding distances in simulated cross-β motifs with low twist rotations (between −10 and 10°). c, Distribution of β-arch sliding distances in β-arch loops from naturally occurring protein structures.

FIG. 8. Distributions of cross-β geometrical parameters obtained from naturally occurring Ig domains and Rosetta folding simulations. a, Median (dotted line) and median absolute deviations for each parameter: distance (10.9±0.8 Å), twist (−32.1±7.7°), roll (12.0±12.2°) and tilt (−4.0±11.1°). Distributions correspond to a set of 275 natural Ig domains with sequence identity below 40%. b, Median (dotted line) and median absolute deviations for each parameter: distance (10.9±1.0 Å), twist (5.7±11.00), roll (9.7±18.0°) and tilt (4.5±9.6°). Distributions correspond to 22,507 cross-β motif models generated by Rosetta folding simulations exploring different combinations of strand lengths (5-7 residues) and frequently observed β-arch loops (3-5 residues). c, Median (dotted line) and median absolute deviations for each parameter: distance (13.7±1.0 Å), twist (21.1±17.2°), roll (10.3±20.4°) and tilt (1.2±11.2). Distributions correspond to 12,335 cross-β motif models generated by Rosetta™ fragment assembly simulations exploring different combinations of strand lengths (5-7 residues) and β-arch helices (3-5 residues).

FIG. 9. β-arch loop twisting. a, Definition of β-arch twist based on the dihedral angle formed between the α-carbons Cα (i−2), Cα (i), Cα (j) and Cα (j+2); where i and j correspond to the residues preceding and following the β-arch loop. b, Distributions of β-arch twist values for loops with frequently observed ABEGO torsion bins forming cross-β motifs in Rosetta™ folding simulations; sampling both positive and negative rotations.

FIG. 10. β-arch helices favoring cross-β motifs obtained from Rosetta folding simulations. The 10 most frequently observed loop-helix-loop ABEGO patterns of each possible sidechain orientation are shown. Frequencies are calculated as the total number of counts across β-arches from all generated cross-β motifs by Rosetta™ folding simulations with a sequence-independent model. Cross-β motif examples for the most frequently observed ABEGO pattern of each sidechain orientation is shown. Most ABEGO patterns have a “B” torsion in the residue preceding the helix, which is typically observed at the start of α-helices in general as it provides N-terminal hydrogen bond capping.

FIG. 11. Structural diversity of the designed proteins. Uniform manifold approximation and projection (UMAP) analysis of computationally designed (“dIGs”) and naturally occurring (“Natives”) Ig domains based on pairwise distances calculated as the TM-score. Experimentally tested designs are shown in orange. The designs broadly sample a structural space distinct from natural Ig proteins.

FIG. 12. Biochemical characterization of the dIG8 design. a, Far-ultraviolet circular dichroism spectra (25° C., 95° C.). b, Thermal denaturation monitored at 220 nm by circular dichroism. The design denatures at temperatures above 95° C. c, SEC-MALS analysis. The protein is monodispersed and has an estimated molecular weight of 16.6 kDa, which lies between that corresponding to the theoretical monomer (10.3 kDa) and dimer (20.6 kDa). The protein includes the thrombin cleavage site and the hexa-histidine purification tag, which adds 2.3 kDa to the design. d, Chemical denaturation with guanidine hydrochloride monitored at 220 nm by circular dichroism. The cooperative unfolding transition indicates that the protein is well-folded. All experiments were carried out with PBS buffer.

FIG. 13. Biochemical characterization of the dIG7 and dIG21 designs. a, Far-ultraviolet circular dichroism spectra (25° C., 75° C., 95° C.). b, SEC-MALS analysis. dIG7 and dIG21 are monodispersed and have estimated molecular weights of 23.4 and 10.1 KDa, which correspond to dimer and monomer respectively. All experiments were carried out with PBS buffer.

FIG. 14. Size-exclusion chromatography combined with multi-angle scattering data. dIG8-CC has a predicted molecular weight between that corresponding to the monomer and dimer, suggesting an equilibrium between both states. dIG14 and other representative designs are predicted to be dimers in solution. Samples were prepared in 20 mM Tris·HCl, 150 mM sodium chloride, pH 7.5.

FIG. 15. Structures predicted for design dIG14 by Rosetta ab initio folding simulations, RoseTTAFold and AlphaFold. Rosetta ab initio folding simulations revealed that the pairing between β-strands 3 and 6 has two conformational states very close in energy, one as designed and the other as observed in the crystal structure. Accurate deep-learning structure prediction with RoseTTAFold™ and AlphaFold™ predict the register shift as designed and with high confidence, which disagrees with the experimental structure. None of the methods predict the C-terminal strand flip out as observed in the crystal, but all predict a conformational rearrangement of the designed β-arch helix; overall pointing to ongoing challenges in predicting structures with multiple possible conformational states and the need of combining folding simulations with deep-learning approaches to verify the accuracy of the designs.

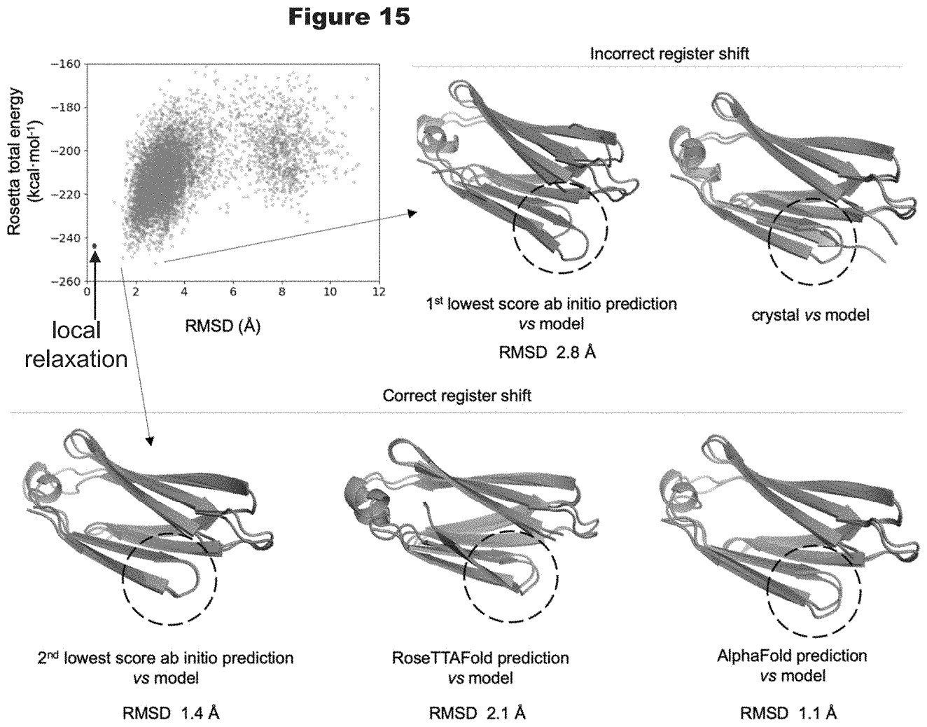

FIG. 16. Naturally occurring protein structures most similar to design dIG8-CC. The closest structural analog with experimental structure available (PDB id: 1CVR) was identified by a TM-align search over a curated dataset of immunoglobulin-like domains as identified by SCOP (those under the Ig β-sandwich fold classification and with X-ray structure resolution <2.5 Å). Closest structural analogues in the AlphaFold™ Protein Structure Database with a confident or highly-confident prediction (pLDDT>70) are also shown (AF-A0A0R0G453). Normalized™-scores are indicated in parentheses.

FIG. 17. Docking calculations on the dIG8-CC homodimer interface. a, Docking calculations of two dIG8-CC monomers using ambiguous restraints between terminal edge strands recapitulate the parallel interface observed in the crystal structure (left). Docking restrained toward the opposite edge predict dimer orientations with disrupted edge-to-edge strand pairing and worse docking scores (right); overall supporting that the terminal edge strands are more dimerization-prone. b, the crystal dimer interface is formed primarily by hydrophobic (top) and salt bridge (bottom) interactions. c, Docking calculations for single-point mutants replacing interface hydrophobics by lysine or glutamate, both of which are known to efficiently disrupt edge-to-edge interfaces as inward-pointing charged residues. All mutants are effective in disrupting the native interface. Some mutants flip the dimer orientation to form antiparallel interfaces with diminished backbone hydrogen-bonded strand pairing and overall higher docking scores. The lowest-score decoy for the most populated cluster of each simulations is shown. Docking scores, calculated with the HADDOCK™ docking software, are provided.

FIG. 18. The dIG14 crystal structure suggests a route to design single-chain Ig dimers through edge-to-edge interfaces. a, Fusing the two 6-stranded Ig domains involved in the dIG14 dimer interface (left) with a short GG linker enables formation of a 12-stranded single-chain Ig dimer. The design model (right) was built by loop insertion between the two chains of the crystal structure. b, Best AlphaFold™ (AF) predicted structure observed from different views. In the center, the AF model is superimposed with the design model with a Cα-RMSD 0.5 Å. c, pLDDT values for the best AlphaFold™ model reveals an overall high-confidence prediction. The dotted vertical line separates the two fused monomers. d, There is a large energy gap between the conformations sampled by Rosetta™ ab initio folding simulations and the designed single-chain dimer structure. Since this is a relatively large protein for ab initio structure prediction (140 amino acids), achieving near-native sampling remains difficult. e, Size-exclusion chromatogram of the expressed and purified single-chain dimer, which is monodisperse and elutes at 13.4 mL as expected for a protein in this size range.

FIG. 19. Protein purification for crystallization studies. Representative final size-exclusion chromatograms of dIG8-CC (a) and dIG14 (c). Retention volumes in mL are indicated above the respective peak. Subsequent SDS-PAGE analysis of dIG8-CC (b) and dIG14 (d) after concentration, with (dIG8-CC) or without (dIG8-CC and dIG14) β-mercaptoethanol (BM).

FIG. 20. Crystal structure of dIG8-CC and functional loop scaffolding. a, Design model of dIG8-CC with a disulfide bridge (spheres) between β-strands 3 and 6. b, SEC-MALS analysis of dIG8-CC estimates a molecular weight between monomer (8.3 kDa) and dimer (16.6 kDa). c, Design model in comparison with the crystal structure with PDB accession code 7SKP (chain C). d, Cross-β motif connections and core sidechain interactions in the design and the crystal structure. The β-arch helix and loop conformations are well preserved across monomer copies in the crystal asymmetric units (insets). e, Crystal homodimer interface by parallel pairing between the two terminal β-strands, which are stabilized through hydrophobic and salt-bridge interactions (inset). f, Computational model of dIG8-CC with a grafted EF-hand motif (design EF61_dIG8-CC, cartoon), showing Tb³⁺ (sphere) bound to EF-hand motif residues (sticks). Tb³⁺ luminescence is sensitized by absorption of light by a proximal tyrosine residue on the EF-hand motif with subsequent fluorescence resonance energy transfer (FRET) to Tb³⁺, resulting in Tb³⁺ luminescence. g, Far-ultraviolet circular dichroism spectra of EF61_dIG8-CC without Tb³⁺ (25° C.; 55° C.; 75° C.; 95° C.). h, Time-resolved luminescence emission spectra in 100 μM Tb³⁺ final concentrations for EF61_dIG8-CC and dIG8-CC at 20 μM. Time-resolved luminescence intensity is given in relative fluorescence units (RFU). i, Tb³⁺ concentration-dependent time-resolved luminescence intensity of 20 μM EF61_dIG8-CC using excitation wavelength λₑₓ=280 nm and emission wavelength λₑₘ=544 nm. Normalized intensities are fit to a one-site binding model by non-linear least squares regression (K_{d}=267 μM).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique. 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In one aspect, the disclosure provides polypeptides comprising the formula X1-X2-X3-X4-X5-X6-X7-X8-X9-X10-X11-X12-X13-X14-X15, wherein:
X1 is optional, and when present comprises 1 2, or 3 residues with loop secondary structure;
X2 comprises 5, 6, 7, or 8 residues with β-strand secondary structure;
X3 comprises 2, 3 or 4 residues with loop secondary structure, forming a β-hairpin tertiary structure motif;
X4 comprises 6, 7, or 8 residues with β-strand secondary structure;
X5 comprises 3, 4, 5 or 6 residues with loop secondary structure, forming a β-arch tertiary structure motif (i.e., a connection between X4 and X6);
X6 comprises 6 or 7 residues with β-strand secondary structure;
X7 comprises 2, 3, or 4 residues with loop secondary structure, forming a β-hairpin tertiary structure motif;
X8 comprises 6 or 7 residues with β-strand secondary structure;
X9 comprises 3, 4 or 5 residues with loop secondary structure, forming a β-arch tertiary structure motif (i.e., a connection between X8 and X10);
X10 comprises 4, 5, 6, 7 or 8 residues with β-strand secondary structure;
X11 comprises one of the following, forming a β-arch tertiary structure motif:
   3, 4, 5, 6, 7, or 8 residues with loop secondary structure; or
   2, 3, or 4 residues with loop secondary structure, followed by 3, 4, 5, or 6 residues with α-helical secondary structure, followed by 1, 2, or 3 residues with loop secondary structure;
X12 comprises 6, 7, or 8 residues with β-strand secondary structure;
X13 comprises 2, 3, or 4 residues with loop secondary structure, forming a β-hairpin tertiary structure motif;
X14 comprises 5, 6, 7, or 8 residues with β-strand secondary structure; and
X15 is optional, and when present comprises 1, 2 or 3 residues with loop secondary structure.

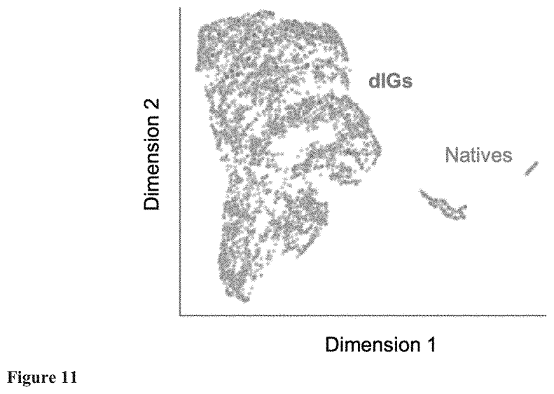

Antibodies and antibody derivatives such as nanobodies contain immunoglobulin-like (Ig) β-sandwich scaffolds which anchor the hypervariable antigen-binding loops and constitute the largest growing class of drugs. The current inventors have developed design rules for the central feature of the Ig fold architecture—the non-local cross-β structure connecting the two β-sheets—and use these to de novo design highly stable seven-stranded Ig domains, confirm their structures through X-ray crystallography, and show they can correctly scaffold functional loops. The high stability of the designs permits grafting functional loops into Ig frameworks with new backbones, exemplified by the EF-hand terbium-binding loop inserted into the C-terminal β-hairpin of dIG8-CC in the examples that follow. Thus, the polypeptides of the disclosure provide antibody-like scaffolds with improved properties. The designs differ substantially from natural Ig domains in global structure, as discussed in the examples (see, for example, FIG. 11 and Table 6).

The X1 and X15 domains are optional and may be present or absent. In various embodiments, neither X1 nor X15 are present; one of X1 or X15 is present (for example, X1 is present; or X15 is present); or X1 and X15 are both present.

X11 forms a β-arch tertiary structure motif, and various embodiments for forming this structure motif are noted above. In one embodiment, X11 comprises 2, 3, or 4 residues with loop secondary structure, followed by 3, 4, 5, or 6 residues with α-helical secondary structure, followed by 1, 2, or 3 residues with loop secondary structure. In another embodiment, X11 comprises a domain structure of 2L5H1L, 2L5H2L, 2L6H1L, 3L4H2L, 3L5H2L, 4L4H2L, 4L4H3L (where “L” stands for loop secondary structure and “H” stands for α-helical secondary structure). For example, the domain structure 2L5H1L means the following: 2L stands for two loop residues, 5H stands for 5 α-helical residues, and 1L stands for 1 loop residue. The meanings of the other domain structures will be understood by those of skill in the art based on the teachings herein.

X5 and X9 also form a β-arch tertiary structure motif, as described above. In various embodiments, 1, 2, or all 3 β-arch motifs (X5, X9, and X11) have atoms involved in hydrogen bonds between (i) two backbone atoms, (ii) one backbone and a sidechain atom, or (iii) two-sidechain atoms. The hydrogen bonds can be within the same β-arch or between the β-arch and other atoms for neighboring domains in the 3-dimensional (3D) structure.

In various other embodiments, 1, 2, 3, 4, 5, 6, 7, or all 8 of the following are true:
(a) X2 forms an antiparallel β-strand pairing with X4;
(b) X4 forms an antiparallel β-strand pairing with X10;
(c) X2, X4, and X10 form a first layer of β-sheets, with X2 and X10 as edge β-strands;
(d) X6 forms an antiparallel β-strand pairing with X8;
(e) X6 forms an antiparallel β-strand pairing with X12;
(f) X12 forms an antiparallel β-strand pairing with X14;
(g) X6, X8, X12 and X14 form a second layer of β-sheets, with X8 and X14 as edge β-strands; and/or
(h) the first layer of 3-sheets and the second layer of 3-sheets form a β-sandwich tertiary structure motif.

In one specific embodiment, all of (a)-(h) are true.

In one embodiment, X4, X6, and X12 comprise alternating hydrophobic and hydrophilic residues. In another embodiment, 1, 2, 3, or all 4 of X2, X8, X10, and X14 comprise alternating hydrophobic and hydrophilic residues. The X4, X6, and X12 domains may form non-edge β-strands, and the X2, X8, X10, and X14 domains may form edge β-strands in, for example, β-sheet and β-sandwich forming embodiments of the disclosure. In these embodiments, the domains as recited may comprise hydrophobic residues in the core (i.e., sidechains pointing toward the interior of the β-sandwich) and polar or charged hydrophilic residues on a solvent-exposed surface (i.e., sidechains pointing toward the exterior of the β-sandwich).

In some embodiments, X4, X6, and X12 independently comprise the amino acid sequence selected from the group consisting of SEQ ID NO: 1-87, as shown in Table 1. In these embodiments, the X4, X6, and X12 domains may each comprise the same amino acid sequence, may each comprise different amino acid sequences, or a combination thereof.

TABLE 1

| | | | | | |
|---|---|---|---|---|---|
| AEIEVRW | AEIHLEF | AEVEVEC | AEVEVEV | AEVRFHY | AEVRIEK |
| SEQ ID | SEQ ID | SEQ ID | SEQ ID | SEQ ID | SEQ ID |
| NO: 1 | NO: 2 | NO: 3 | NO: 4 | NO: 5 | NO: 6 |
| AEVRLEI | ARIEIKV | ARVEFEY | ARVEIEV | ARVEMEV | ARVEVEE |
| SEQ ID | SEQ ID | SEQ ID | SEQ ID | SEQ ID | SEQ ID |
| NO: 7 | NO: 8 | NO: 9 | NO: 10 | NO: 11 | NO: 12 |
| ATLEVEK | ATVHVEK | ATVRIEY | CRVRVTA | EFHVEA | EIEVEV |
| SEQ ID | SEQ ID | SEQ ID | SEQ ID | SEQ ID | SEQ ID |
| NO: 13 | NO: 14 | NO: 15 | NO: 16 | NO: 17 | NO: 18 |
| EVEVEVKA | EVEVTVEY | EVRVEVEM | EYEVEVEN | GKVRIEF | GRLELEY |
| SEQ ID | SEQ ID | SEQ ID | SEQ ID | SEQ ID | SEQ ID |
| NO: 19 | NO: 20 | NO: 21 | NO: 22 | NO: 23 | NO: 24 |
| GRVEFEV | GRVEIKA | GRVEVQV | GRVEVRF | GTIEIEV | GTVEIEV |
| SEQ ID | SEQ ID | SEQ ID | SEQ ID | SEQ ID | SEQ ID |
| NO: 25 | NO: 26 | NO: 27 | NO: 28 | NO: 29 | NO: 30 |
| GTVEVEV | IRFEMTM | IRFEMVM | IRFEVEV | IRVEVEV | KFEFRN |
| SEQ ID | SEQ ID | SEQ ID | SEQ ID | SEQ ID | SEQ ID |
| NO: 31 | NO: 32 | NO: 33 | NO: 34 | NO: 35 | NO: 36 |
| KIEVEN | KIEVRVTN | KMRVRLR | KMRVRLRN | KVEIRVRS | KVEVHVEA |
| SEQ ID | SEQ ID | SEQ ID | SEQ ID | SEQ ID | SEQ ID |
| NO: 37 | NO: 38 | NO: 39 | NO: 40 | NO: 41 | NO: 42 |
| KVEVRN | KVHIRT | KYELRIRN | MRIEVRA | MRVEFTW | QFTIEW |
| SEQ ID | SEQ ID | SEQ ID | SEQ ID | SEQ ID | SEQ ID |
| NO: 43 | NO: 44 | NO: 45 | NO: 46 | NO: 47 | NO: 48 |
| QWEVRIRN | RIEIQIHN | RIRVEVQE | RIRVRN | RLEVEIRN | RLRIHVEV |
| SEQ ID | SEQ ID | SEQ ID | SEQ ID | SEQ ID | SEQ ID |
| NO: 49 | NO: 50 | NO: 51 | NO: 52 | NO: 53 | NO: 54 |
| RVEIEIV | RVEIEN | RVEIEY | RVEVRVEN | RVEVRVQS | RVRLEY |
| SEQ ID | SEQ ID | SEQ ID | SEQ ID | SEQ ID | SEQ ID |
| NO: 55 | NO: 56 | NO: 57 | NO: 58 | NO: 59 | NO: 60 |
| RVRVRN | RYEFRN | RYRLEIRT | TFEVEIRN | TIEFRFEN | TIEIRS |
| SEQ ID | SEQ ID | SEQ ID | SEQ ID | SEQ ID | SEQ ID |
| NO: 61 | NO: 62 | NO: 63 | NO: 64 | NO: 65 | NO: 66 |
| TIHVEVEN | TIQLHQ | TIRVEN | TIRVEVR | TIRVTVEN | TVEVTV |
| SEQ ID | SEQ ID | SEQ ID | SEQ ID | SEQ ID | SEQ ID |
| NO: 67 | NO: 68 | NO: 69 | NO: 70 | NO: 71 | NO: 72 |
| VEVHIKV | VEVRMEK | VEVRVRA | VEVRVRN | VHVEVEI | VRFRVVG |
| SEQ ID | SEQ ID | SEQ ID | SEQ ID | SEQ ID | SEQ ID |
| NO: 73 | NO: 74 | NO: 75 | NO: 76 | NO: 77 | NO: 78 |
| VRFTVEM | VRIRVT | VRITVEV | VRVEVEV | VRVEVKA | VRVEVVY |
| SEQ ID | SEQ ID | SEQ ID | SEQ ID | SEQ ID | SEQ ID |
| NO: 79 | NO: 80 | NO: 81 | NO: 82 | NO: 83 | NO: 84 |
| VRVRVTA | VTLEVEF | YRVEVEN | | | |
| SEQ ID | SEQ ID | SEQ ID | | | |
| NO: 85 | NO: 86 | NO: 87 | | | |

In various other embodiments, 1, 2, 3, or all 4 of X2, X8, X10, and X14 comprise alternating hydrophobic and hydrophilic residues, independently comprising the amino acid sequence selected from the following group consisting of SEQ ID NO:88-123, as shown in Table 2. In these embodiments, the X2, X8, X10, and X14 domains may each comprise the same amino acid sequence, may each comprise different amino acid sequences, or a combination thereof.

TABLE 2

| | | | | | |
|---|---|---|---|---|---|
| AKYELKG | AKYEVEL | ELEI | ELEIRMD | ELELHF | ELEVE |
| SEQ ID | SEQ ID | SEQ ID | SEQ ID | SEQ ID | SEQ ID |
| NO: 88 | NO: 89 | NO: 90 | NO: 91 | NO: 92 | NO: 93 |
| ELRIEF | EVEVTFS | FEVRVQWS | IEVRVD | IEVRVK | KVQLELH |
| SEQ ID | SEQ ID | SEQ ID | SEQ ID | SEQ ID | SEQ ID |
| NO: 94 | NO: 95 | NO: 96 | NO: 97 | NO: 98 | NO: 99 |
| KYSYEYT | LEIR | LEVRME | MEVRVS | MEYR | QMRVEIS |
| SEQ ID | SEQ ID | SEQ ID | SEQ ID | SEQ ID | SEQ ID |
| NO: 100 | NO: 101 | NO: 102 | NO: 103 | NO: 104 | NO: 105 |
| RIEVEVT | RLELHFQ | RLTVEFT | RVEVE | RVRFEFR | SVEVR |
| SEQ ID | SEQ ID | SEQ ID | SEQ ID | SEQ ID | SEQ ID |
| NO: 106 | NO: 107 | NO: 108 | NO: 109 | NO: 110 | NO: 111 |
| TIEVQ | TLRLRG | TVRVEF | VEIRYK | VEVRIS | VEVRLE |
| SEQ ID | SEQ ID | SEQ ID | SEQ ID | SEQ ID | SEQ ID |
| NO: 112 | NO: 113 | NO: 114 | NO: 115 | NO: 116 | NO: 117 |
| VEVRVEFE | VEVRVTY | VRLRFTV | VRVEV | VRWTWRIS | WEVRVRWK |
| SEQ ID | SEQ ID | SEQ ID | SEQ ID | SEQ ID | SEQ ID |
| NO: 118 | NO: 119 | NO: 120 | NO: 121 | NO: 122 | NO: 123 |

In another embodiment X2, X8, X10, and X14 each comprise at least one polar amino acid residue selected from Arg, Lys, Glu, Gln, and His. The X2, X8, X10, and X14 domains may form edge β-strands in, for example, β-sheet and β-sandwich forming embodiments of the disclosure. In these embodiments, the domains comprise at least one inward-facing (i.e., sidechains pointing toward the interior of the β-sandwich) polar amino acid (e.g., Arg, Lys, Glu, Gln, His).

In further embodiments, X2, X8, X10, and X14 independently comprise the amino acid sequence selected from the group consisting of SEQ ID NO: 88-203. The amino acid sequence of SEQ ID NO: 124-203 are provided in Table 3. In this embodiment, the X2, X8, X10, and X14 domains may each comprise the same amino acid sequence, may each comprise different amino acid sequences, or a combination thereof.

TABLE 3

| | | | | | |
|---|---|---|---|---|---|
| EEVEIEV | EKRTVTV | EKRTYEFQ | EKVRTRYR | EKYEVRI | EKYRFQL |
| SEQ ID | SEQ ID | SEQ ID | SEQ ID | SEQ ID | SEQ ID |
| NO: 124 | NO: 125 | NO: 126 | NO: 127 | NO: 128 | NO: 129 |
| EKYTYT | ERIEVQ | ERIQFEA | ETLEVE | ETREYTV | ETVEVEV |
| SEQ ID | SEQ ID | SEQ ID | SEQ ID | SEQ ID | SEQ ID |
| NO: 130 | NO: 131 | NO: 132 | NO: 133 | NO: 134 | NO: 135 |
| EVRVQEK | FRVEVREK | IHVELRKE | IRIEVRSS | IRVEIEK | KKFTYTV |
| SEQ ID | SEQ ID | SEQ ID | SEQ ID | SEQ ID | SEQ ID |
| NO: 136 | NO: 137 | NO: 138 | NO: 139 | NO: 140 | NO: 141 |
| KKLEYQY | KKYEYTV | KREERTF | KREEYHM | KRERWRFR | KRERYTL |
| SEQ ID | SEQ ID | SEQ ID | SEQ ID | SEQ ID | SEQ ID |
| NO: 142 | NO: 143 | NO: 144 | NO: 145 | NO: 146 | NO: 147 |
| KRETYTV | KRHEVHL | KRYEYTA | KTEETQ | LEVRRK | LEVRVRKS |
| SEQ ID | SEQ ID | SEQ ID | SEQ ID | SEQ ID | SEQ ID |
| NO: 148 | NO: 149 | NO: 150 | NO: 151 | NO: 152 | NO: 153 |
| QREEYRM | QREEYTL | QRERYHV | QRETYTA | QRETYTV | QRFTFEFT |
| SEQ ID | SEQ ID | SEQ ID | SEQ ID | SEQ ID | SEQ ID |
| NO: 154 | NO: 155 | NO: 156 | NO: 157 | NO: 158 | NO: 159 |
| QRFTYRY | QRQHFQ | QRQRYQ | QRREYTG | QRREYTV | QRYEFTA |
| SEQ ID | SEQ ID | SEQ ID | SEQ ID | SEQ ID | SEQ ID |
| NO: 160 | NO: 161 | NO: 162 | NO: 163 | NO: 164 | NO: 165 |
| QRYEYHM | QRYRFE | QRYTFEVR | QRYTYEVR | QRYTYTL | QTVEY |
| SEQ ID | SEQ ID | SEQ ID | SEQ ID | SEQ ID | SEQ ID |
| NO: 166 | NO: 167 | NO: 168 | NO: 169 | NO: 170 | NO: 171 |
| REYRYKL | RKIETELT | RLRYRTK | RREEYTA | RREEYTL | RRERYEA |
| SEQ ID | SEQ ID | SEQ ID | SEQ ID | SEQ ID | SEQ ID |
| NO: 172 | NO: 173 | NO: 174 | NO: 175 | NO: 176 | NO: 177 |
| RRERYTA | RRETYTV | RRFTYTG | RRYEVR | SELRVT | SEVHVRFE |
| SEQ ID | SEQ ID | SEQ ID | SEQ ID | SEQ ID | SEQ ID |
| NO: 178 | NO: 179 | NO: 180 | NO: 181 | NO: 182 | NO: 183 |
| STKWRFE | TEMRVEI | TEVRVEQ | TLETTA | TQYRYEFE | TRIEFE |
| SEQ ID | SEQ ID | SEQ ID | SEQ ID | SEQ ID | SEQ ID |
| NO: 184 | NO: 185 | NO: 186 | NO: 187 | NO: 188 | NO: 189 |
| TRYEYE | TTETYTV | TTITVEGT | TTYEYTV | TTYQRTI | TTYRYEV |
| SEQ ID | SEQ ID | SEQ ID | SEQ ID | SEQ ID | SEQ ID |
| NO: 190 | NO: 191 | NO: 192 | NO: 193 | NO: 194 | NO: 195 |
| TTYRYRL | VEFRLREE | VEIEVRTK | VEVRIREE | VEVRIRKN | VEVRITEK |
| SEQ ID | SEQ ID | SEQ ID | SEQ ID | SEQ ID | SEQ ID |
| NO: 196 | NO: 197 | NO: 198 | NO: 199 | NO: 200 | NO: 201 |
| VEVRQS | VRTSYTM | | | | |
| SEQ ID | SEQ ID | | | | |
| NO: 202 | NO: 203 | | | | |

In other embodiments, X2, X4, X6, X8, X10, X12, and X14 independently comprise an amino acid sequence selected from SEQ ID NO: 1-203. In this embodiment, the X2, X4, X6, X8, X10, X12, and X14 domains may each comprise the same amino acid sequence, may each comprise different amino acid sequences, or a combination thereof.

In other embodiments, X5, X9, and X11 comprise (i) at least one polar amino acid selected from Asn, Ser, Thr, Glu, and Gin in the domain or in the residue immediately preceding or following the domain, where the polar residue is involved in at least one hydrogen bond between (i) two backbone atoms, (ii) one backbone and a sidechain atom, or (iii) two-sidechain atoms, and (iv) a glycine or proline residue. The X5, X9, and X11 domains form β-arches and, for example, in β-sheet and β-sandwich forming embodiments of the disclosure, these domains comprise at least one inward-facing polar amino acid forming hydrogen bonds (Asn, Ser, Thr, Glu, Gin) and a glycine or proline. The polar amino acid can also be in the residue preceding or following the β-arch loop. For instance, in X5 this would mean that the last residue of X4 or the first of X6 could be a polar amino acid involved in a hydrogen bond. The hydrogen bond(s) can be within the same β-arch or between the β-arch and other atoms from neighboring domains in the 3D structure.

In another embodiment, X5, X9, and X11 independently comprise the amino acid sequence selected from the group consisting of SEQ ID NO:236-286, and DGP, DRP, EGP, NGP, NKG, NPG, PPG, and RGE. In this embodiment, the X5, X9, and X11 domains may each comprise the same amino acid sequence, may each comprise different amino acid sequences, or a combination thereof.

TABLE 4

| | | | | |
|---|---|---|---|---|
| AGARE | ANDPHKFNRP | APPGQD | VSPEELKNL | VSTAEKQGI |
| SEQ ID | SEQ ID | SEQ ID | SEQ ID | SEQ ID |
| NO: 236 | NO: 237 | NO: 238 | NO: 239 | NO: 240 |
| DSSTP | VQPGA | GEPGQD | GGPGE | GPPGQD |
| SEQ ID | SEQ ID | SEQ ID | SEQ ID | SEQ ID |
| NO: 241 | NO: 242 | NO: 243 | NO: 244 | NO: 245 |
| GRPGE | GTDKP | GTDRP | GVPPGG | IRAR |
| SEQ ID | SEQ ID | SEQ ID | SEQ ID | SEQ ID |
| NO: 246 | NO: 247 | NO: 248 | NO: 249 | NO: 250 |
| ISPEEAKN | ISPEELKNA | KSDRP | KSSQP | LSPEQQNN |
| SEQ ID | SEQ ID | SEQ ID | SEQ ID | SEQ ID |
| NO: 251 | NO: 252 | NO: 253 | NO: 254 | NO: 255 |
| LTKP | NDSSTP | VPLRQE | VPNGR | NNSDRP |
| SEQ ID | SEQ ID | SEQ ID | SEQ ID | SEQ ID |
| NO: 256 | NO: 257 | NO: 258 | NO: 259 | NO: 260 |
| TTDPRELKNA | NPGD | NPGE | NPGEE | NPGG |
| SEQ ID | SEQ ID | SEQ ID | SEQ ID | SEQ ID |
| NO: 261 | NO: 262 | NO: 263 | NO: 264 | NO: 265 |
| NPGT | NSDKP | NSDQP | NSDRP | NSSQP |
| SEQ ID | SEQ ID | SEQ ID | SEQ ID | SEQ ID |
| NO: 266 | NO: 267 | NO: 268 | NO: 269 | NO: 270 |
| PGKRP | TSPKP | PPGAPP | PPGS | PPGT |
| SEQ ID | SEQ ID | SEQ ID | SEQ ID | SEQ ID |
| NO: 271 | NO: 272 | NO: 273 | NO: 274 | NO: 275 |
| PPNSK | QSDRP | RAPSP | TSPDSSQNKGL | RGKTP |
| SEQ ID | SEQ ID | SEQ ID | SEQ ID | SEQ ID |
| NO: 276 | NO: 277 | NO: 278 | NO: 279 | NO: 280 |
| RSDQP | RSDRP | RSSQP | RVPNAR | SPSDDRQKRP |
| SEQ ID | SEQ ID | SEQ ID | SEQ ID | SEQ ID |
| NO: 281 | NO: 282 | NO: 283 | NO: 284 | NO: 285 |
| TADQP | | | | |
| SEQ ID | | | | |
| NO: 286 | | | | |

In some embodiments, the X3, X7, and X13 domains each comprise at least one glycine residue. In other embodiments, the X3, X7, and X13 domains independently comprise the amino acid sequence selected from the group consisting of APGT (SEQ ID NO:287), DG, EG, GD, GE, GG, GK, GKGV (SEQ ID NO:288), GN, KGNR (SEQ ID NO:289), KNN, NG, PG, and RGDS (SEQ ID NO:290). In this embodiment, the X3, X7, and X13 domains may each comprise the same amino acid sequence, may each comprise different amino acid sequences, or a combination thereof.

In one embodiment of all embodiments herein, at least two non-contiguous β-strands include a cysteine residue, wherein the at least two non-contiguous β-strand cysteine residues are capable of forming a disulfide bond. “Non-contiguous” β-strands are those that are not contiguous in the primary amino acid sequence (i.e.: X2 and X4 are contiguous β-strands; X2 and X6 are non-contiguous β-strands; etc.). The meaning of non-contiguous β-strands will be understood by those of skill in the art based on the teachings herein. In another embodiment, the first residue of the X6 domain and the last residue of the X12 domain are cysteine residues capable of forming a disulfide bond. In a further embodiment, the polypeptide comprises a disulfide bond between non-contiguous β-strands, such as a disulfide bond formed between the X6 and X12 domains.

In a further embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical, not including any functional domain insertions, to the amino acid sequence selected from the group consisting of SEQ ID NO:204-235. The sequences are shown in Table 5, and include annotations in the form X1+X2+X3+X4+X5+X6+X7+X8+X9+X10+X11+X12+X13+X14+X15, showing the position of the domains within the primary amino acid sequence.

TABLE 5

| SEQ | | |
|---|---|---|
| ID | Design | |
| NO | name | Amino acid sequence |
| 204 | dIG1 | TVEVRIRKNGNEYEVEVENRSDRPAEVRFHYDGTTETYTVPPGT |
| | | RLRYRTKLTKPMRIEVRAGNTTYEYTVS |
| | | T\|VEVRIRKN\|GN\|EYEVEVEN\|RSDRP\|AEVRFHY\|DG\|TTET |
| | | YTV\|PPGT\|RLRYRTK\|LTKP\|MRIEVRA\|GN\|TTYEYTV\|S |
| 205 | dIG2 | EIHVELRKEGDRVEVRVENRSSQPGTVEIEVDGQRYEFTANPGE |
| | | RIQFEARGKTPVRVEVVYGNTTYRYEVR |
| | | E\|IHVELRKE\|GD\|RVEVRVEN\|RSSQP\|GTVEIEV\|DG\|QRYE |
| | | FTA\|NPG\|ERIQFEA\|RGKTP\|VRVEVVY\|GN\|TTYRYEV\|R |
| 206 | dIG3 | RVRVEVKNNKIEVENNSDQPAEIHLEFGGRRFTYTGNKGERIEV |
| | | QISPEEAKNARIEIKVGDKKLEYQYH |
| | | R\|VRVEV\|KNN\|KIEVEN\|NSDQP\|AEIHLEF\|GG\|RRFTYTG\| |
| | | NKG\|ERIEVQ\|ISPEEAKN\|ARIEIKV\|GD\|KKLEYQY\|H |
| 207 | dIG4 | RVEVRISGNTIRVENRSDRPARVEFEYGGRREEYTAPPGSELRV |
| | | TISPEELKNARVEIEYGGQRYRFEVT |
| | | R\|VEVRIS\|GN\|TIRVEN\|RSDRP\|ARVEFEY\|GG\|RREEYTA\| |
| | | PPG\|SELRVT\|ISPEELKNA\|RVEIEY\|GG\|QRYRFE\|VT |
| 208 | dIG5 | KIRIEVRSSGNTIHVEVENNSDRPVRIRVTAPGTTLETTANPGE |
| | | RVRFEFRGVPPGGEVEVEVKAGDEKVRTRYRS |
| | | K\|IRIEVRSS\|GN\|TIHVEVEN\|NSDRP\|VRIRVT\|APGT\|TLE |
| | | TTA\|NPGE\|RVRFEFR\|GVPPGG\|EVEVEVKA\|GD\|EKVRTRY |
| | | R\|S |
| 209 | dIG6 | TVEVRITEKNGQWEVRIRNRSSQPARVEVEEGGRREEYTLNPGD |
| | | ELELHFTSPKPVRITVEVGGQRYTYTLR |
| | | T\|VEVRITEK\|NG\|QWEVRIRN\|RSSQP\|ARVEVEE\|GG\|RREE |
| | | YTL\|NPGD\|ELELHF\|TSPKP\|VRITVEV\|GG\|QRYTYTL\|R |
| 210 | dIG7 | RMEVRVSNGRVEIENKSSQPGRVEVRFNGKRYEYTANPGERVEV |
| | | EVSPEELKNLRVRLEYDGKTEETQYS |
| | | R\|MEVRVS\|NG\|RVEIEN\|KSSQP\|GRVEVRF\|NG\|KRYEYTA\| |
| | | NPGE\|RVEVE\|VSPEELKNL\|RVRLEY\|DG\|KTEETQ\|YS |
| 211 | dIG8 | RIEVRVDNGRVRVRNGTDRPVRVRVTAGGETREYTVNPGTELEV |
| | | ELSPEQQNNAEVEVEVGNEKYRFQLG |
| | | R\|IEVRVD\|NG\|RVRVRN\|GTDRP\|VRVRVTA\|GG\|ETREYTV\| |
| | | NPGT\|ELEVE\|LSPEQQNN\|AEVEVEV\|GN\|EKYRFQL\|G |
| 212 | dIG9 | SIRVEIEKRGDSYRVEVENRSDQPAEIEVRWNGRRERYEANKGE |
| | | TVEVEVRAPSPVEVRVRAGNTEVRVEQR |
| | | S\|IRVEIEK\|RGDS\|YRVEVEN\|RSDQP\|AEIEVRW\|NG\|RRER |
| | | YEA\|NKG\|ETVEVEV\|RAPSP\|VEVRVRA\|GN\|TEVRVEQ\|R |
| 213 | dIG10 | RVEVRISGNTIEIRSEGPGRLELEYNGQREEYTLNPGTRIEFEG |
| | | RPGEEVRVEVEMNGQRYTFEVRF |
| | | R\|VEVRIS\|GN\|TIEIRS\|EGP\|GRLELEY\|NG\|QREEYTL\|NP |
| | | G\|TRIEFE\|GRPGE\|EVRVEVEM\|NG\|QRYTFEVR\|F |
| 214 | dIG11 | RLEVRMEGKKVEVRNNSDRPMRVEFTWNGQRERYHVNPGETLEV |
| | | EVQPGARVEVRVQSGDTQYRYEFEL |
| | | R\|LEVRME\|GK\|KVEVRN\|NSDRP\|MRVEFTW\|NG\|QRERYHV\| |
| | | NPG\|ETLEVE\|VQPGA\|RVEVRVQS\|GD\|TQYRYEFE\|L |
| 215 | dIG12 | SLEVRVRKSGNTFEVEIRNKSDRPAEVRLEIGGRRETYTVPPGS |
| | | TLRLRGPGKRPGRVEIKAGDAKYEVELR |
| | | S\|LEVRVRKS\|GN\|TFEVEIRN\|KSDRP\|AEVRLEI\|GG\|RRET |
| | | YTV\|PPGS\|TLRLRG\|PGKRP\|GRVEIKA\|GD\|AKYEVEL\|R |
| 216 | dIG13 | YVEIRYKGEKVHIRTNGPVTLEVEFEGKRERYTLNPGEELEIRI |
| | | RARRIRVEVQEGDRKIETELTF |
| | | Y\|VEIRYK\|GE\|KVHIRT\|NGP\|VTLEVEF\|EG\|KRERYTL\|NP |
| | | GEE\|LEIR\|IRAR\|RIRVEVQE\|GD\|RKIETELT\|F |
| 217 | dIG14 | RVEVRVEFEGDKMRVRLRNDSSTPVEVHIKVGDEKRTVTVNPGE |
| | | EVEVTFSANDPHKFNRPQFTIEWGGQRQHFQHH |
| | | R\|VEVRVEFE\|GD\|KMRVRLRN\|DSSTP\|VEVHIKV\|GD\|EKRT |
| | | VTV\|NPGE\|EVEVTFS\|ANDPHKFNRP\|QFTIEW\|GG\|QRQHF |
| | | Q\|HH |
| 218 | dIG15 | RPKVQLELHGNKMRVRLRNDSSTPVEVHIKVGDEKRTVTVNPGE |
| | | EVEVTFSTTDPRELKNATIQLHQGDQTVEYRVD |
| | | RP\|KVQLELH\|GN\|KMRVRLR\|NDSSTP\|VEVHIKV\|GD\|EKRT |
| | | VTV\|NPGE\|EVEVTFS\|TTDPRELKNA\|TIQLHQ\|GD\|QTVEY\| |
| | | RVD |
| 219 | dIG16 | EVEIEVRTKNGKIEVRVTNRSDRPVEVRMEKGGQRETYTAPPGS |
| | | TVRVEFSPSDDRQKRPTVEVTVNGRRYEVRVH |
| | | E\|VEIEVRTK\|NG\|KIEVRVTN\|RSDRP\|VEVRMEK\|GG\|QRET |
| | | YTA\|PPGS\|TVRVEF\|SPSDDRQKRP\|TVEVTV\|NG\|RRYEVR\| |
| | | VH |
| 220 | dIG17 | RVEFRLREEGDRYRLEIRTDRPGTIEIEVNGRRERYTANPGTTI |
| | | TVEGTRGEEVEVTVEYDGKRERWRFRM |
| | | R\|VEFRLREE\|GD\|RYRLEIRT\|DRP\|GTIEIEV\|NG\|RRERYT |
| | | A\|NPG\|TTITVEGT\|RGE\|EVEVTVEY\|DG\|KRERWRFR\|M |
| 221 | dIG18 | RVRWTWRISGNTIEFRFENNSDRPARVEIEVDGQRREYTVNPGE |
| | | RLELHFQAGAREIRVEVEVGKEKYEVRIRF |
| | | R\|VRWTWRIS\|GN\|TIEFRFEN\|NSDRP\|ARVEIEV\|DG\|QRRE |
| | | YTV\|NPGE\|RLELHFQ\|AGARE\|IRVEVEV\|GK\|EKYEVRI\|RF |
| 222 | dIG19 | RVEVRIREEGDKYELRIRNRSDRPAEVRIEKGGKRETYTVNPGE |
| | | ELRIEFPPGAPPGRVEVQVGDKKYEYTVK |
| | | R\|VEVRIREE\|GD\|KYELRIRN\|RSDRP\|AEVRIEK\|GG\|KRET |
| | | YTV\|NPGE\|ELRIEF\|PPGAPP\|GRVEVQV\|GD\|KKYEYTV\|K |
| 223 | dIG20 | VVEVRLEGERIRVRNNSDRPATVHVEKDGQRETYTVNPGEELEI |
| | | TSPDSSQNKGLRLRIHVEVNGQRFTFEFTM |
| | | VIVEVRLE\|GE\|RIRVRN\|NSDRP\|ATVHVEK\|DG\|QRETYTV\| |
| | | NPGE\|ELEI\|TSPDSSQNKGL\|RLRIHVEV\|NG\|QRFTFEFT\|M |
| 224 | dIG21 | SIEVRVKGDRYEFRNNSDKPATLEVEKNGKREEYHMNPGESVEV |
| | | RGEPGQDIRFEMVMEGTTYRYRLS |
| | | S\|IEVRVK\|GD\|RYEFRN\|NSDKP\|ATLEVEK\|NG\|KREEYHM\| |
| | | NPGE\|SVEVR\|GEPGQD\|IRFEMVM\|EG\|TTYRYRL\|S |
| 225 | dIG22 | SIEVRVKGDRYEFRNNSDKPATLEVEKNGKREEYHMNPGESVEV |
| | | RGPPGQDIRFEMTMDGTTYRYRLS |
| | | S\|IEVRVK\|GD\|RYEFRN\|NSDKP\|ATLEVEK\|NG\|KREEYHM\| |
| | | NPGE\|SVEVR\|GPPGQD\|IRFEMTM\|DG\|TTYRYRL\|S |
| 226 | dIG23 | DLEVRRKDGKFEFRNNSDKPATLEVEKDGQREEYRMNPGETIEV |
| | | QAPPGQDVRFTVEMPGREYRYKLD |
| | | D\|LEVRRK\|DG\|KFEFRN\|NSDKP\|ATLEVEK\|DG\|QREEYRM\| |
| | | NPGE\|TIEVQ\|APPGQD\|VRFTVEM\|PG\|REYRYKL\|D |
| 227 | dIG24 | TFEVRVQWSGNTIRVTVENQSDRPATVRIEYGNTTYQRTINPGD |
| | | RLTVEFTGGPGEVHVEVEINGKREERTFTK |
| | | T\|FEVRVQWS\|GN\|TIRVTVEN\|QSDRP\|ATVRIEY\|GN\|TTYQ |
| | | RTI\|NPGD\|RLTVEFT\|GGPGE\|VHVEVEI\|NG\|KREERTF\|TK |
| 228 | dIG25 | EVQMRVEISGDTIRVEVRNNSDRPGRVEFEVGGVRTSYTMNPGE |
| | | RIEVEVTVSTAEKQGIKVEVHVEAGDEKRTYEFQM |
| | | EV\|QMRVEIS\|GD\|TIRVEVR\|NNSDRP\|GRVEFEV\|GG\|VRTS |
| | | YTM\|NPGE\|RIEVEVT\|VSTAEKQGI\|KVEVHVEA\|GD\|EKRTY |
| | | EFQ\|M |
| 229 | dIG26 | RVEVRVQEKNGKVEIRVRSDGPVRVEVEVGGQRREYTGNPGEEV |
| | | EIEVTADQPVRVEVKAGDKKFTYTVSE |
| | | RV\|EVRVQEK\|NG\|KVEIRVRS\|DGP\|VRVEVEV\|GG\|QRREYT |
| | | G\|NPG\|EEVEIEV\|TADQP\|VRVEVKA\|GD\|KKFTYTV\|SE |
| 230 | dIG27 | MFRVEVREKNGRVEVRVENRSDRPGTVEVEVGGVRLRFTVNPGE |
| | | ELEIRMDVPNGRRVEIEIVGKGVKYSYEYTV |
| | | M\|FRVEVREK\|NG\|RVEVRVEN\|RSDRP\|GTVEVEV\|GG\|VRLR |
| | | FTV\|NPGE\|ELEIRMD\|VPNGR\|RVEIEIV\|GKGV\|KYSYEYT\| |
| | | V |
| 231 | dIG28 | SWEVRVRWKNGRLEVEIRNNSSQPGKVRIEFDGKRHEVHLNPGE |
| | | STKWRFENPGGEFHVEAGKEKYTYTV |
| | | S\|WEVRVRWK\|NG\|RLEVEIRN\|NSSQP\|GKVRIEF\|DG\|KRHE |
| | | VHL\|NPGE\|STKWRFE\|NPGG\|EFHVEA\|GK\|EKYTYT\|V |
| 232 | dIG29 | RVEVRQSGNTIEIRSEGPGRLELEYNGQREEYTLNPGTRYEYEG |
| | | RPGEEVRVEVEMNGQRYTYEVRS |
| | | R\|VEVRQS\|GN\|TIEIRS\|EGP\|GRLELEY\|NG\|QREEYTL\|NP |
| | | G\|TRYEYE\|GRPGE\|EVRVEVEM\|NG\|QRYTYEVR\|S |
| 233 | dIG30 | RSEVHVRFEGERIEIQIHNGTDKPARVEMEVNGQRYEYHMPPNS |
| | | KMEYRVPLRQEIRFEVEVGGQRFTYRYTS |
| | | R\|SEVHVRFE\|GE\|RIEIQIHN\|GTDKP\|ARVEMEV\|NG\|QRYE |
| | | YHM\|PPNSK\|MEYR\|VPLRQE\|IRFEVEV\|GG\|QRFTYRY\|TS |
| 234 | dIG31 | RVEVRVTYKGNRVEVRVRNNSDRPVRFRVVGPGAKYELKGNPGT |
| | | EMRVEIRVPNAREIEVEVNGQRQRYQM |
| | | R\|VEVRVTY\|KGNR\|VEVRVRN\|NSDRP\|VRFRVVG\|PG\|AKYE |
| | | LKG\|NPG\|TEMRVEI\|RVPNAR\|EIEVEV\|NG\|QRQRYQ\|M |
| 235 | dIG8-CC | RIEVRVDNGRVRVRNGTDRPCRVRVTAGGETREYTVNPGTELEV |
| | | ELSPEQQNNAEVEVECGNEKYRFQLG |
| | | R\|IEVRVD\|NG\|RVRVRN\|GTDRP\|CRVRVTA\|GG\|ETREYTV\| |
| | | NPGT\|ELEVE\|LSPEQQNN\|AEVEVEC\|GN\|EKYRFQL\|G |

The “|” symbol indicates the demarcation of the domains X1-X15 within the primary amino acid sequence.

In one embodiment, amino acid substitutions relative to the reference polypeptide are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Proteins comprising conservative amino acid substitutions can be tested in any one of the assays described herein to confirm that a desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gin (Q); (3) acidic: Asp (D), Glu (E); (4) basic: Lys (K), Arg (R), His (H). Alternatively, naturally occurring residues can be divided into groups based on common sidechain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, glycine and/or proline residues are maintained relative to the reference polypeptide.

In another embodiment, the polypeptides of any embodiment disclosed herein further comprise one or more functional domains inserted into the polypeptide. The high stability of the polypeptides of the disclosure permits grafting functional loops into Ig frameworks with new backbones, exemplified by the EF-hand terbium-binding loop inserted into the C-terminal β-hairpin of dIG8-CC in the examples.

As used herein, a functional domain is any polypeptide domain that provides any functional benefit to the polypeptide as suitable for an intended use. Non-limiting examples of such functional domains include detectable polypeptides, small-molecule binding motifs, metal ion binding motifs, polypeptide binding motifs, nucleic acid binding motifs, substrate binding motifs including SpyTag™ or SpyCatcher™ motifs, green fluorescent proteins and variants thereof, luminescent proteins and variants thereof, antibodies, other scaffolding proteins (i.e., combinations of polypeptides of the present disclosure, scaffolding proteins for higher-order protein assemblies, etc.) or enzymes.

In one embodiment, one or more functional domains are inserted into domain X1 and/or X15. By way of non-limiting example, X1 and X15 may be covalently fused to chemical linkers (i.e., for attachment to substrates) and/or directly to one or more functional domain. In other embodiments, one or more functional domains are inserted into domain X3, X5, X7, X9, X11, and/or X13. In this embodiment, domain X3, X5, X7, X9, X11, and/or X13 may be functionalized/diversified by insertion of one or more functional domain or non-functional loops (i.e., polyglycine). This implies a loop or domain with any function in combination with polypeptide linkers that may be used for incorporation onto the protein (i.e., within a β-arch or β-hairpin motif, such that flanking loop residues surrounding the inserted functional motif may be used as linkers connecting the functional motif to the dIG protein). One or more functional motifs may be inserted into any one or more of X3, X5, X7, X9, X11, and/or X13 in any combination. In this embodiment, the polypeptide may be designed to mimic the function of complementarity-determining regions (CDRs).

In one embodiment, any one or more domain further comprises an attached fluorophore, chemiluminescent compound, or reactive moieties for “click” chemistry. A non-limiting example of a polypeptide containing insertions of functional domains comprises the amino acid sequence of SEQ ID NO:300, with the functional domain (EF-hand calcium-binding motif) shown in bold font and underlining, with added linkers also in bold font.

“EF61_dIG8-CC” sequence:

(SEQ ID NO: 300)
RIEVRVDNGRVRVRNGTDRPCRVRVTAGGETREYTVNPGTELEVELSPEQ
QNNAEVEVECTVDDKDGDGYISAAEAAVEKYRFQLG

And with sequence annotation of the form

(SEQ ID NO: 300)
X1 + X2 + X3 + X4 + X5 + X6 + X7 + X8 + X9 + X10 +
X11 + X12 + X13 + X14 + X15:
R + IEVRVD + NG + RVRVRN + GTDRP + CRVRVTA + GG +
ETREYTV + NPGT + ELEVE + LSPEQQNN + AEVEVEC +
TVDDKDGDGYISAAEAAV + EKYRFQL + G

As used throughout the present application, the term “polypeptide” is used in its broadest sense to refer to a sequence of subunit D- or L-amino acids, including canonical and non-canonical amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another embodiment, the disclosure comprises multimers, comprising 2, 3, 4, 5, 6, or more copies of the polypeptide of any embodiment or combinations herein. The multimers could be formed either by interaction of dIG monomers having the full sequence (from X1 to X15) or without X1, X14 and/or X15. For example, removing the last β-strand can also allow formation of stable multimers. Thus, in various embodiments, one or more polypeptides in the multimer are deleted for domains X1, X14, and/or X15. In another embodiment, one or more polypeptides in the multimer are deleted for domains X14 and X15.

The polypeptides in the multimer may all be identical, may all be different, or a combination thereof. The multimer may be any suitable multimer. In one embodiment, the multimer comprises a dimer. The multimer may be formed in any manner suitable for an intended use. In one embodiment, the multimers such as dimers may be formed by β-strand pairing of edge strands to form intermolecular protein-protein interfaces. In another embodiment, the multimers such as dimers may be covalently fused via polypeptide linker into a single chain protein, such that the dimers of the polypeptide scaffolds may be formed by β-strand pairing of edge strands to form intramolecular protein-protein interfaces. One non-limiting example of a single-chain dimer comprises the amino acid sequence of SEQ ID NO:301.

“dIG14-scdim” sequence (underlined is a short diglycine polypeptide linker):

(SEQ ID NO 301)
GRVEVRVEFEGDKMRVRLRNDSSTPVEVHIKVGDEKRTVTVNPGEEVEVT
FSANDPHKFNRPQFTIEWGGGGRVEVRVEFEGDKMRVRLRNDSSTPVEVH
IKVGDEKRTVTVNPGEEVEVTFSANDPHKFNRPQFTIEWG

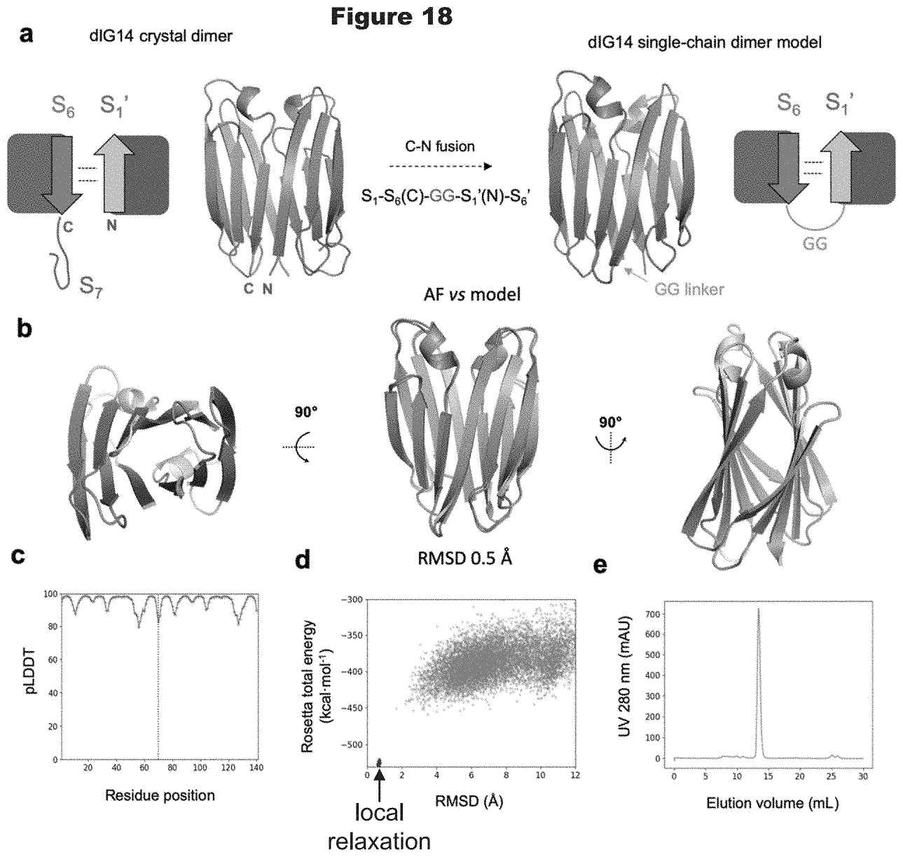

In this embodiment, dIG14-scdim is present in two copies, but deleted for domains X14 and X15 (the first monomer contains X1 to X13 and is linked to a second monomer also containing domains with X1-X13). Data supporting this particular design is in current FIG. 18. Other specific embodiments are provided below as SEQ ID NO: 291-299.

Single-chain dimers: Dimers fused by a polypeptide linker (underlined).
The dIG8-scdim sequences were designed following a very similar strategy to that already described for dIG14-scdim (Example 2).

dIG14-scdim
(SEQ ID NO: 291)
GRVEVRVEFEGDKMRVRLRNDSSTPVEVHIKVGDE
KRTVTVNPGEEVEVTFSANDPHKFNRPQFTIEWGG
GGRVEVRVEFEGDKMRVRLRNDSSTPVEVHIKVGD
EKRTVTVNPGEEVEVTFSANDPHKFNRPQFTIEWG
dIG8-scdim1
(SEQ ID NO: 292)
TETIEVRVDNGRVRVRNGTDRPIRVRVTAGGETRE
YTVNPGTELEVELSPEQQNQAIVVIHIGNEVFMFV
LARDEEWVKRAEKLAEELNVRILVIVLNGRVRVRN
GTDRPMRVRVTAGGETREYTVNPGTELEVELSPEQ
QNNAEVEVEVGNRKWRFQLG
dIG8-scdim2
(SEQ ID NO: 293)
RIEVRVDNGRVRVRNGTDRPIRVRVTAGGETREYT
VNPGTELEVELSPEQQNQAIVVVHIGNRVFMWVLA
RDEEWVKRAEKLAEELNVRILVIVLNGRVRVRNGT
DRPMRVRVTAGGETREYTVNPGTELEVELSPEQQN
NAEVEVEVGNEKWRFQLG
dIG8-scdim3
(SEQ ID NO: 294)
TQTIEVRVDNGRVRVRNGTDRPIRVRVTAGGETRE
YTVNPGTELEVELSPEQQNNAMVEVQVGNEIVFFI
LAHNEELAKRWWEEAKQRAKILVMVLNGRVRVRNG
TDRPMRVRVTAGGETREYTVNPGTELEVELSPEQQ
NNAEVEVEVGNNKYRFQLG

### Functional Designs

The full insertion is in bold (linkers+calcium binding motif). The binding motif sequence is underlined. These sequences contain 1 or 2 simultaneous functional insertions.

dIG14-scdim + EF3a
(SEQ ID NO: 295)
GRVEVRVEFEGDKMRVRLRNDSSTPVEVHIKVGDE
KRTVTVNPGEEVEVTFSANDPHKFNRPQFTIEWKD
DKDGDGYISAAEKGRVEVRVEFEGDKMRVRLRNDS
STPVEVHIKVGDEKRTVTVNPGEEVEVTFSANDPH
KFNRPQFTIEWG
dIG14-scdim + EF1a + EF3a
(SEQ ID NO: 296)
GRVEVRVEFEPIEDKDGDGYISAAEAAAAKMRVRL
RNDSSTPVEVHIKVGDEKRTVTVNPGEEVEVTFSA
NDPHKFNRPQFTIEWKDDKDGDGYISAAEKGRVEV
RVEFEGDKMRVRLRNDSSTPVEVHIKVGDEKRTVT
VNPGEEVEVTFSANDPHKFNRPQFTIEWG
dIG14-scdim + EF2e + EF3a
(SEQ ID NO: 297)
GRVEVRVEFEGDKMRVRLRNDSSTPVEVHIKVGGD
KDGDGYISAAEAKDAEKDAEKRTVTVNPGEEVEVT
FSANDPHKFNRPQFTIEWKDDKDGDGYISAAEKGR
VEVRVEFEGDKMRVRLRNDSSTPVEVHIKVGDEKR
TVTVNPGEEVEVTFSANDPHKFNRPQFTIEWG
dIG14-scdim + EF1a + EF4
(SEQ ID NO: 298)
GRVEVRVEFEPIEDKDGDGYISAAEAAAAKMRVRL
RNDSSTPVEVHIKVGDEKRTVTVNPGEEVEVTFSA
NDPHKFNRPQFTIEWGGGGRVEVRVEFEPIEDKDG
DGYISAAEAAAAKMRVRLRNDSSTPVEVHIKVGDE
KRTVTVNPGEEVEVTFSANDPHKFNRPQFTIEWG
dIG14-scdim + EF3a + EF4
(SEQ ID NO: 299)
GRVEVRVEFEGDKMRVRLRNDSSTPVEVHIKVGDE
KRTVTVNPGEEVEVTFSANDPHKFNRPQFTIEWKD
DKDGDGYISAAEKGRVEVRVEFEPIEDKDGDGYIS
AAEAAAAKMRVRLRNDSSTPVEVHIKVGDEKRTVT
VNPGEEVEVTFSANDPHKFNRPQFTIEWG

Thus, in further embodiments, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical, not including any functional domain insertions, to the amino acid sequence selected from the group consisting of SEQ ID NO: 291-301.

In another aspect the disclosure provides nucleic acids encoding the polypeptide or multimer of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise polypeptides, multimers, nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another embodiment, the disclosure provides pharmaceutical compositions comprising:
(a) the polypeptide, multimer, nucleic acid, expression vector, or host cell of any preceding claim; and
(b) a pharmaceutically acceptable carrier.

In one embodiment, the pharmaceutical composition comprises a polypeptide or multimer that includes a therapeutic functional domain.

In another aspect, the disclosure provides methods for using the polypeptide, multimer, nucleic acid, expression vector, host cell, or pharmaceutical composition of any embodiment or combination of embodiments herein, including but not limited to scaffolding functional domains for any suitable use, diagnostics, therapeutics, and biosensing.

In another aspect, the disclosure provides methods for designing the polypeptides or multimers of any embodiment herein, comprising any method as disclosed in the examples.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### Example 1

### Abstract

Antibodies and antibody derivatives such as nanobodies contain immunoglobulin-like (Ig) β-sandwich scaffolds which anchor the hypervariable antigen-binding loops and constitute the largest growing class of drugs. Current engineering strategies for this class of compounds rely on naturally existing Ig frameworks, which can be hard to modify and have limitations in manufacturability, designability and range of action. Here we develop design rules for the central feature of the Ig fold architecture—the non-local cross-β structure connecting the two β-sheets—and use these to de novo design highly stable seven-stranded Ig domains, confirm their structures through X-ray crystallography, and show they can correctly scaffold functional loops. Our approach provides a new class of antibody-like scaffolds with tailored structures and superior biophysical properties.

### Introduction

To date, approaches to engineering antibodies rely on naturally occurring Ig backbone frameworks, and mainly focus on optimizing the antigen-binding loops and/or multimeric formats for improving targeting efficiency or biophysical properties.

### Results

### Principles for Designing Cross-β Motifs

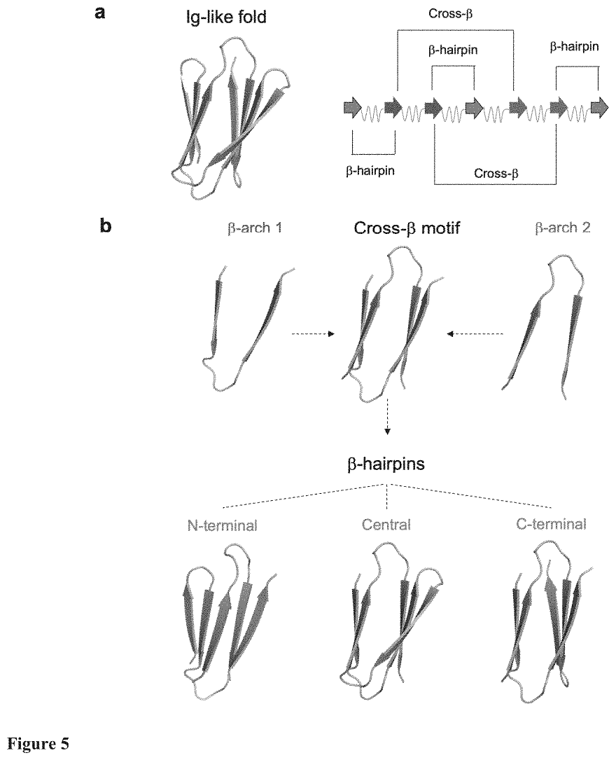

We began by investigating how the structural requirements associated with cross-β motifs constrain the geometry of the two β-arches connecting the β-strands. Since β-arch connections have four possible sidechain orientation patterns (8) (“⬆⬆”, “⬆⬇”, “⬇⬆” and “⬇⬇”) depending on whether the C_{α}C_{β} vector of the β-strand residues preceding and following the connection point to the concave (“⬇”) or convex (“⬆”) face of the arch, there are sixteen possible cross-β motif connection orientations in total. For example, the “⬆⬆/⬇⬇” cross-β connection orientation means that the first and second β-arch connections have the “⬆⬆” and “⬇⬇” orientations, respectively. Due to the alternating pleating of β-strands, the cross-β connection orientation and the length of the β-strands in the two β-sheets are strongly coupled: if paired β-strands have no register shift, they must be odd-numbered in four cross-β orientations, even-numbered in four cross-β orientations, and odd-numbered in one of the two β-sheets and even-numbered in the other in the remaining eight cases. Guided by this principle, we studied the efficiency in forming cross-β motifs of highly structured β-arch connections; too flexible β-arches can hinder folding as they increase the protein contact order (14)—the average sequence separation between contacting residues—which slows down folding. The cross-β motif is the highest contact order part of the Ig fold architecture, and thus the rate of formation of this structure likely determines the overall rate of folding and thus contributes to the balance between folding and aggregation; once the cross-β is formed, folding is likely completed rapidly as the remaining β-hairpins are sequence-local (FIG. 5).

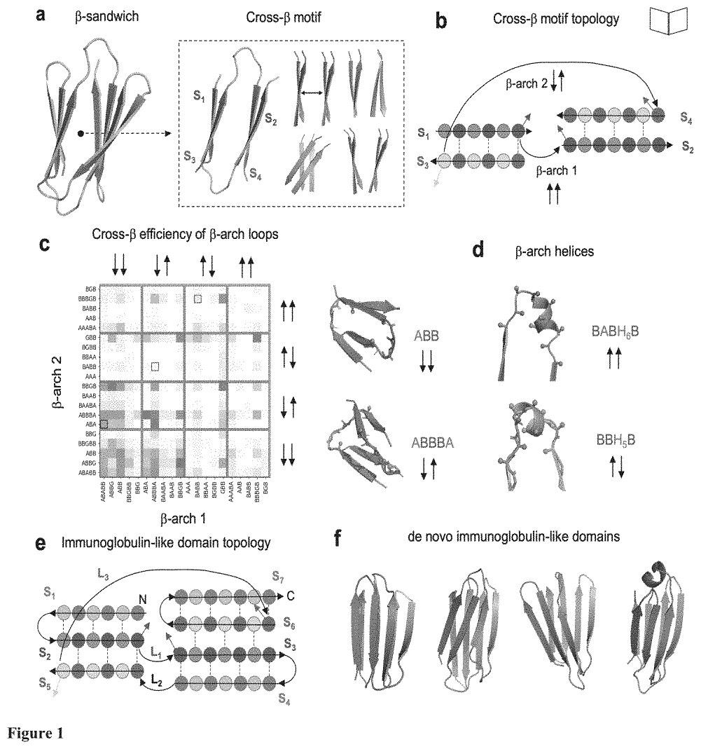

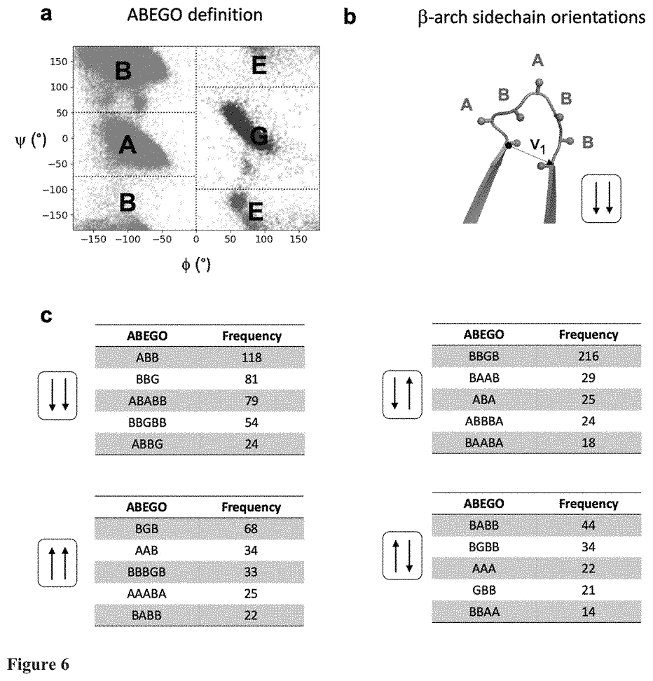

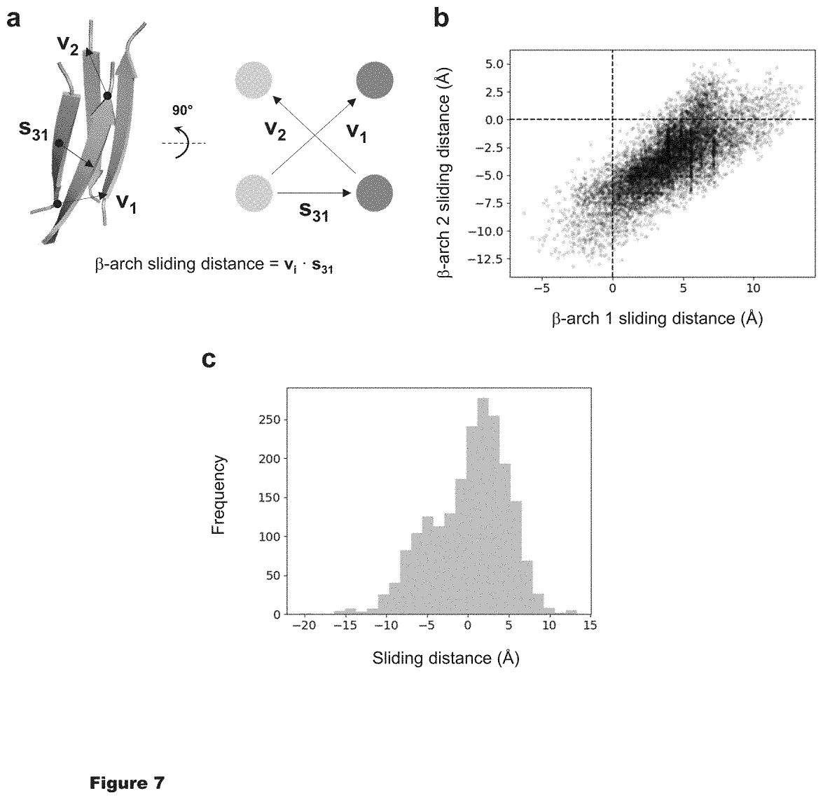

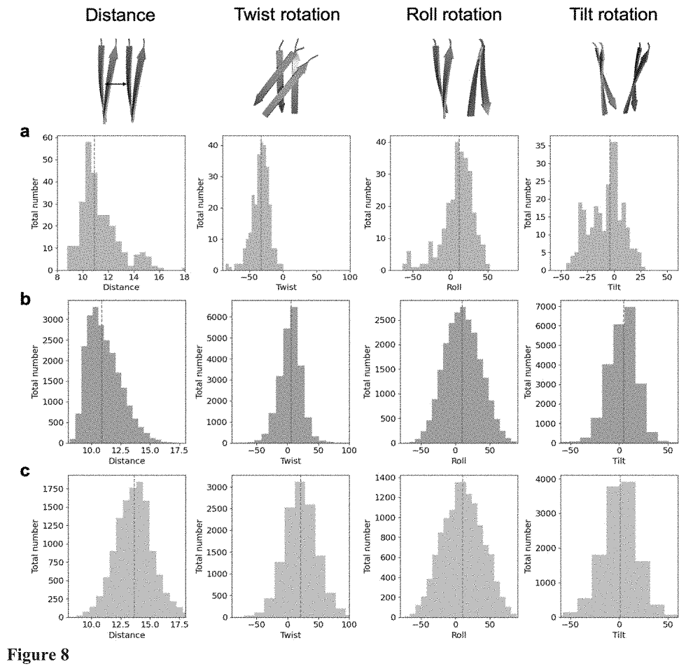

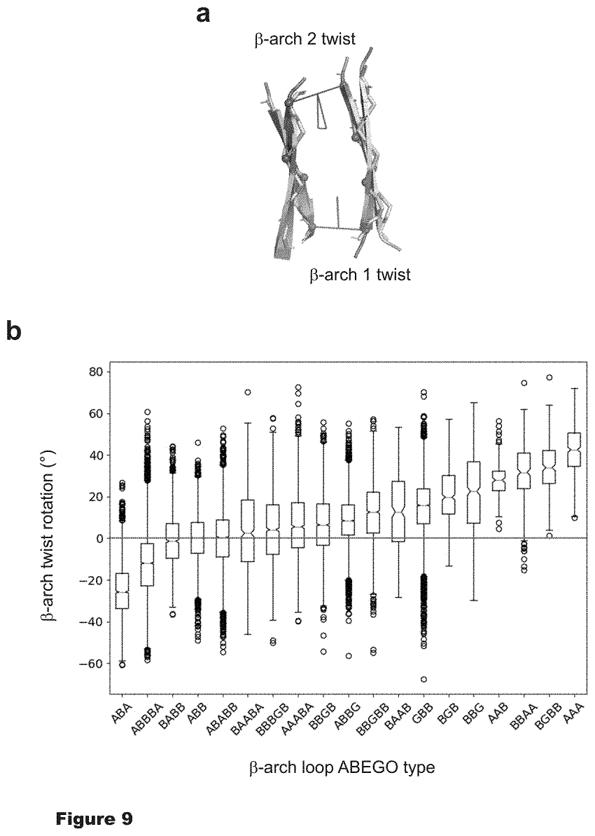

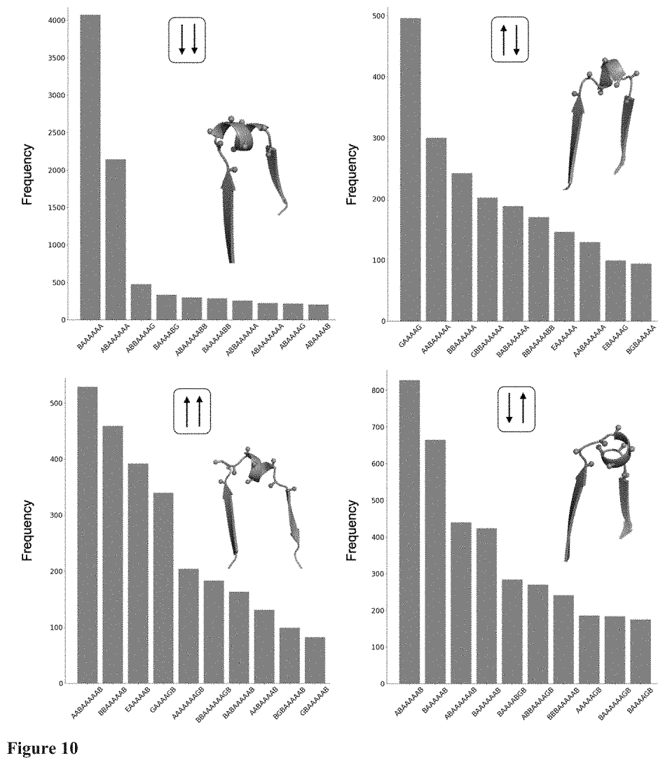

We generated cross-β motifs exploring combinations of short β-arch loops frequently observed in naturally occurring proteins and spanning the sixteen possible sidechain orientations (FIG. 6), along with β-strand length, using Rosetta™ folding simulations with a sequence-independent model (7, 15) biased by the ABEGO torsion bins (see FIG. 6a for a definition) describing loop backbone geometry (16) (FIG. 1c). For cross-β motifs to form, the geometry of the two β-arch loops must allow the concerted spanning of the proper distance along the β-sheet pairing direction and along an axis connecting the two opposing β-sheets so that the two following β-strands cross and switch the order of β-strand pairing in the opposite β-sheet (FIG. 7). Multiple pairs of β-arch loops with the same or different ABEGO torsion bins were found to fulfill these geometrical requirements (FIG. 1c), with sampled ranges of cross-β geometrical parameter values similar to or broader than in naturally occurring Ig domains (FIG. 8). For example, β-arch loops “ABB” and “ABBBA” strongly favor cross-β motifs but with twist rotations (FIG. 9) in opposite directions (FIG. 1c, right). We next explored the efficiency of short α-helices (spanning 4-6 residues) connecting the two β-strands through short loops (of 1-3 residues) which we refer to as “β-arch helices”. For cross-β motifs formed with β-arch helices, we identified efficient loop-helix-loop patterns (i.e. helix length together with adjacent loop ABEGO-types) for the four possible β-arch sidechain orientations (FIG. 10). Overall, the formation and structure of cross-β motifs can in this way be encoded by combining β-arch loops and/or β-arch helices of specific geometry with β-strands compatible in terms of length and sidechain orientations.

### Computational Design of Ig Domains

Based on these rules relating β-arch connections with cross-β motifs, we de novo designed seven-stranded Ig topologies (FIG. 1e, f). We generated protein backbones by Rosetta™ Monte Carlo fragment assembly using blueprints (7, 15) specifying secondary structures and ABEGO torsion bins, together with hydrogen-bond restraints specifying β-strand pairing. We explored combinations of β-strand lengths (between 5 and 8 residues) and register shifts between paired β-strands 3 and 6 (between 0 and 2). β-arches 1 and 3 are those involved in the cross-β motif, and their connections were built with loop ABEGO-types having high cross-β propensity, as described above. We reasoned that β-arch helices may fit better in β-arch 3 than in β-arch 1 (FIG. 1e), which by construction is more embedded in the core (as it stacks with β-arch loop 2 forming a β-arcade), and also explored topology combinations combining β-arch 1 loops with β-arch 3 helices. The three β-hairpin loops were designed with two residues for proper control of the orientation between the two paired β-strands according to the ββ-rule (7). Those topology combinations with β-strand lengths incompatible with the expected sidechain orientations of each β-arch and β-hairpin connection were automatically discarded. We then carried out Rosetta™ sequence design calculations (17, 18) for the generated backbones. Loops were designed using consensus sequence profiles derived from fragments with the same ABEGO backbone torsions. Cysteines were not allowed during design to avoid dependence of correct folding on disulfide bond formation (in contrast to most natural Ig domains). To minimize risk of edge-to-edge interactions promoting aggregation, at least one inward-facing polar or charged amino acid (TQKRE; SEQ ID NO:302) (19) was incorporated into each solvent-exposed edge β-strand. Sequences were ranked based on energy and sidechain packing metrics, as well as local sequence-structure compatibility assessed by 9-mer fragment quality analysis (4). Folding of the top-ranked designs was quickly screened by biased forward folding simulations (5), and those with near-native sampling were subjected to Rosetta™ ab initio folding simulations from the extended chain (20). The extent to which the designed sequences encode the designed structures was also assessed through AlphaFold™ (21) or RoseTTAFold™ (22) structure prediction calculations (see below).

### Biochemical and Structural Characterization of the Designs

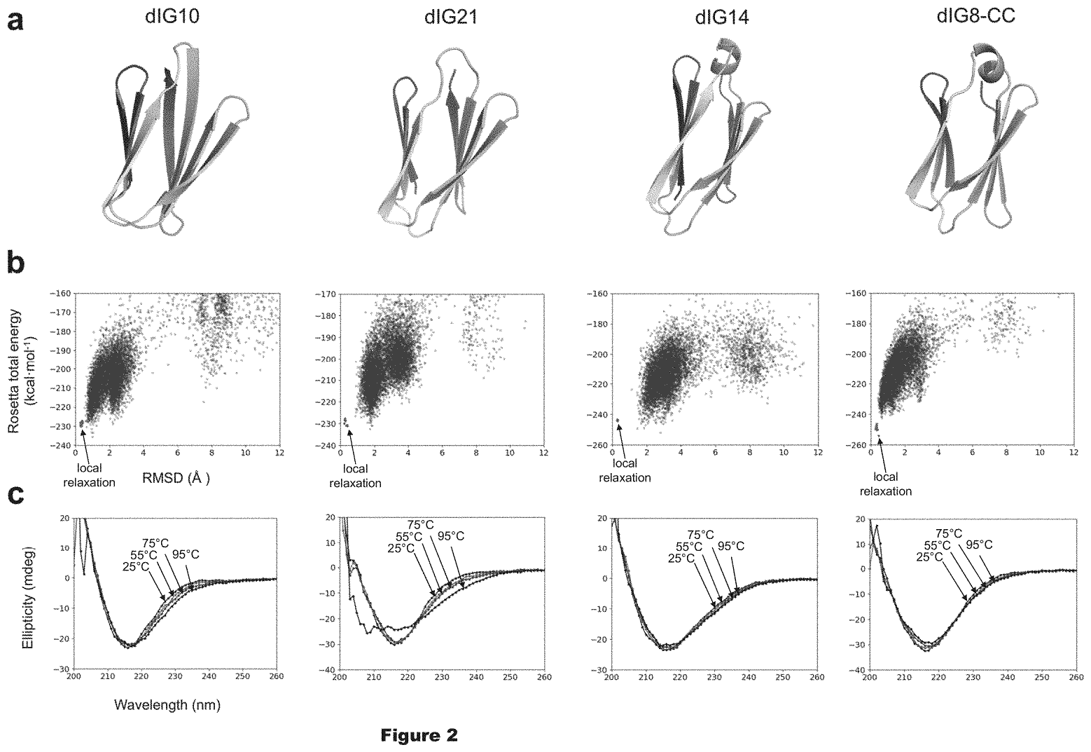

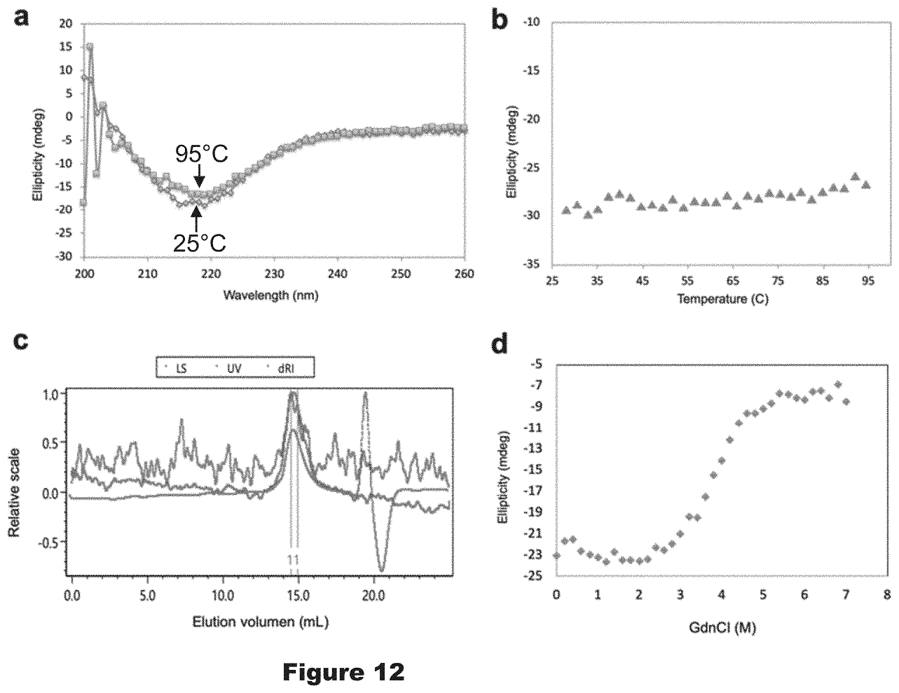

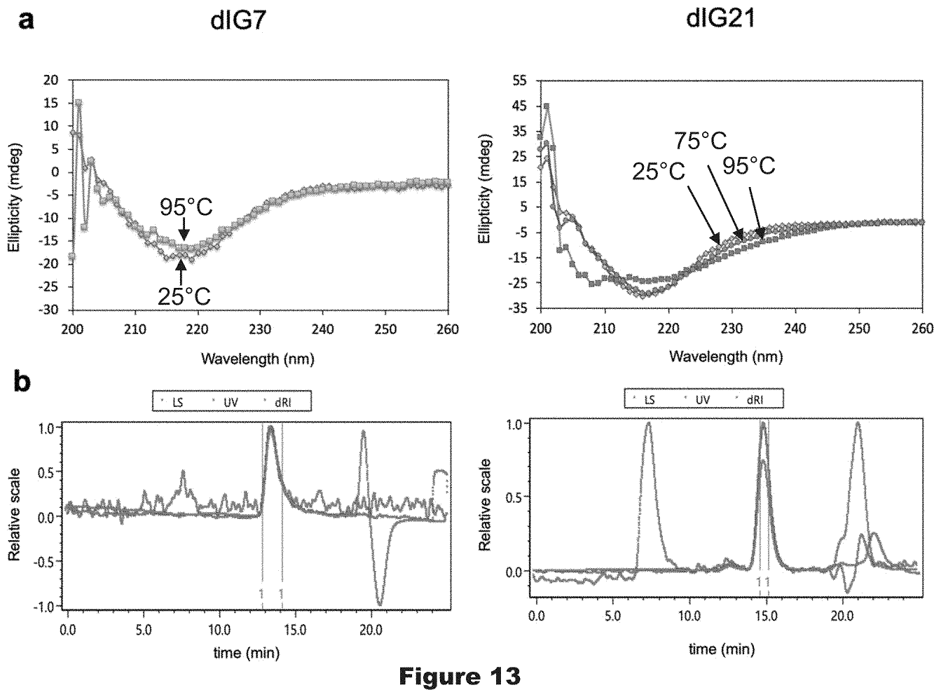

For experimental characterization, we selected 31 designs predicted to fold correctly by ab initio structure prediction (FIG. 2a, b); 29 of which had AlphaFold™ or RoseTTAFold™ predicted models with pLDDT>80 and Cα-root mean square deviations (Cα-RMSDs)<2 Å to the design models. The designed sequences contain between 66 and 79 amino acids and are unrelated to naturally occurring sequences, with Blast (23) (E-values >0.1) and more sensitive sequence-profile searches (24, 25) finding very weak or no remote homology (E-values >0.003). The designs also differ substantially from natural Ig domains in global structure (FIG. 11), and cross-β twist rotation (close to zero, which are infrequent in natural Ig domains; Table 6). We obtained synthetic genes encoding for the designed amino acid sequences (design names are dIGn, where “dIG” stands for “designed ImmunoGlobulin” and “n” is the design number). We expressed them in Escherichia coli, and purified them by affinity and size-exclusion chromatography. Overall, 24 designs were present in the soluble fraction and 8 were monodisperse, had far-UV circular dichroism spectra compatible with an all-β protein structure, and were thermostable (Tₘ>95° C., except for dIG21 with Tₘ>75° C.) (FIG. 2c, Table 7 and FIG. 12-14). In size-exclusion chromatography combined with multi-angle light scattering (SEC-MALS), five designs were dimeric, one monomeric (dIG21) and another one (dIG8) was found in equilibrium between monomer and dimer (FIG. 11-13).

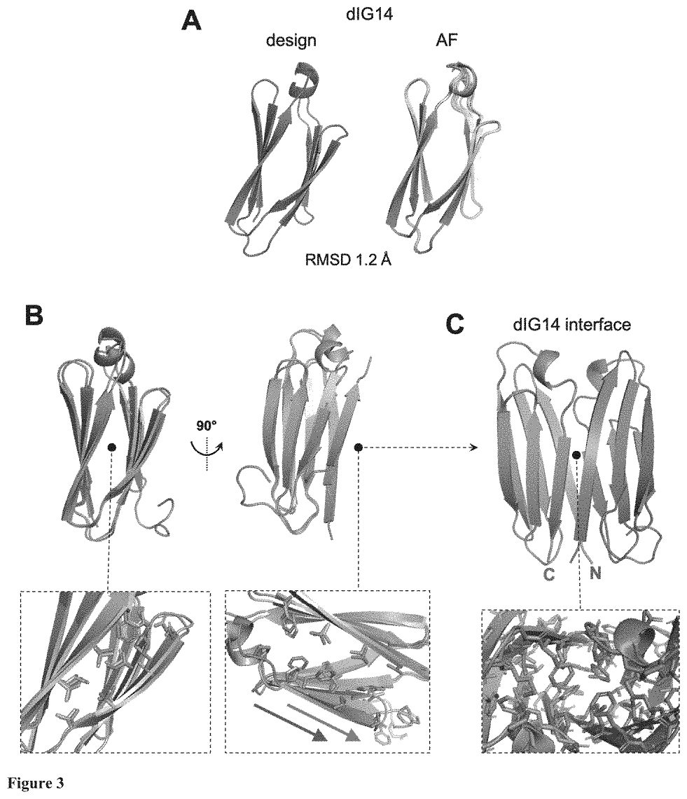

For one of the designs that was dimeric in solution (FIG. 3a) and well-folded by NMR (data not shown), dIG14, we solved a crystal structure at 2.4 Å resolution (FIG. 3b, Table 8) and found it was in excellent agreement with the computational model over the first five β-strands and their connections (Cα-RMSD of 0.7 Å). By contrast, the C-terminal region had three main differences: β-arch helix 3 was found in a different orientation, the register between paired β-strands 6 and 3 shifted by two β-strand positions (FIG. 3b, bottom right), and the C-terminal β-strand flipped out of the structure, being disordered. This conformational difference exposed the protein core and formed an edge-to-edge dimer interface mediated by antiparallel contacts between β-strands 1 and 6, overall forming a 12-stranded β-sandwich (FIG. 3c). AlphaFold™ and RoseTTAFold™ predictions recapitulated our design model (FIG. 3a), but with lower pLDDT values in the β-arch helix. Rosetta™ ab initio folding simulations, instead, sampled both the crystal and designed conformations, and were found very close in energy; suggesting that protein folding energy landscapes should be

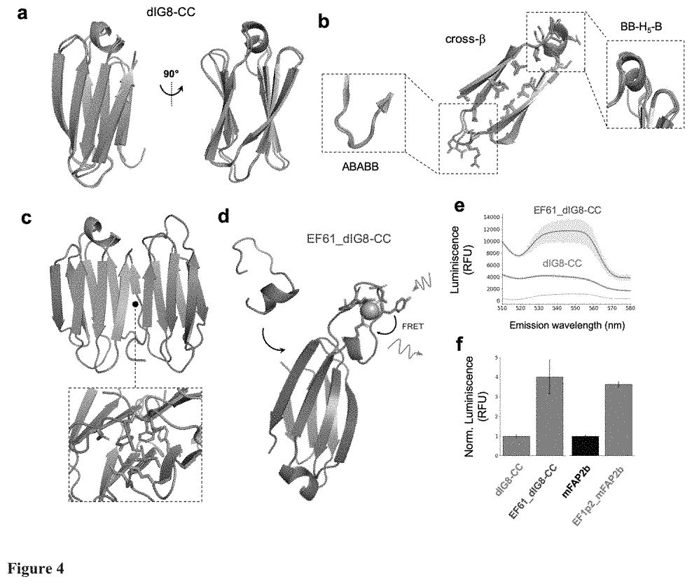

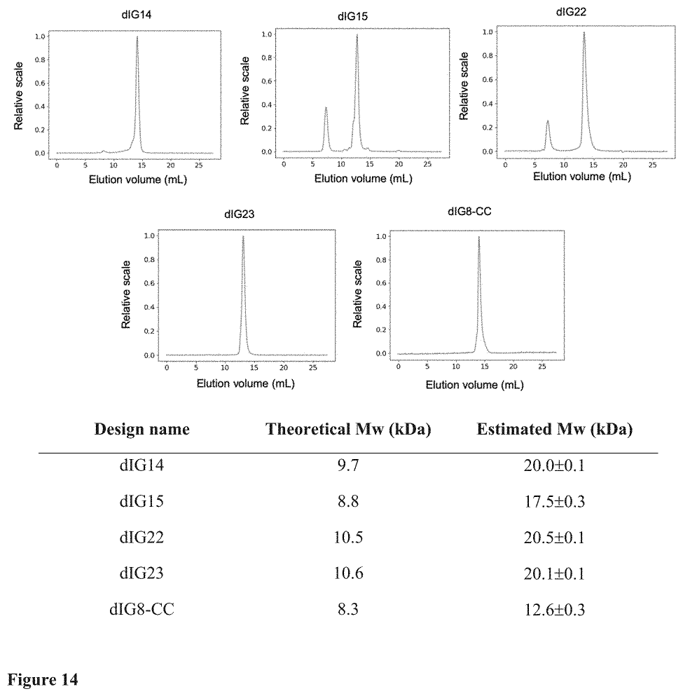

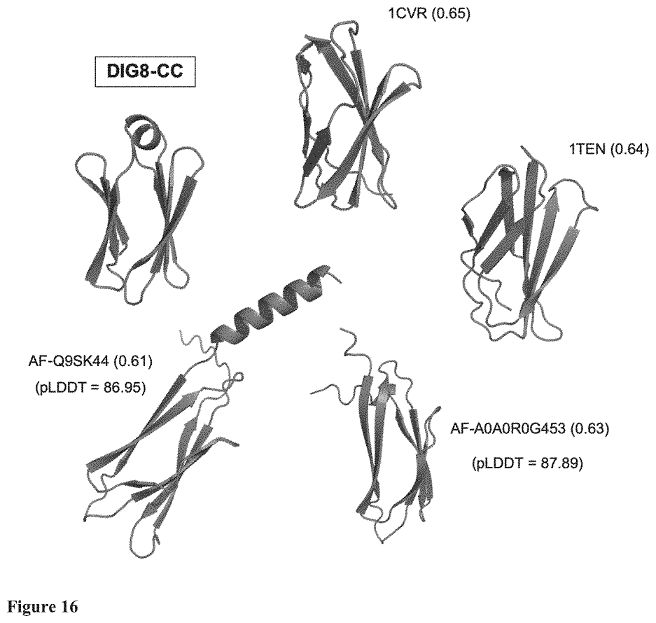

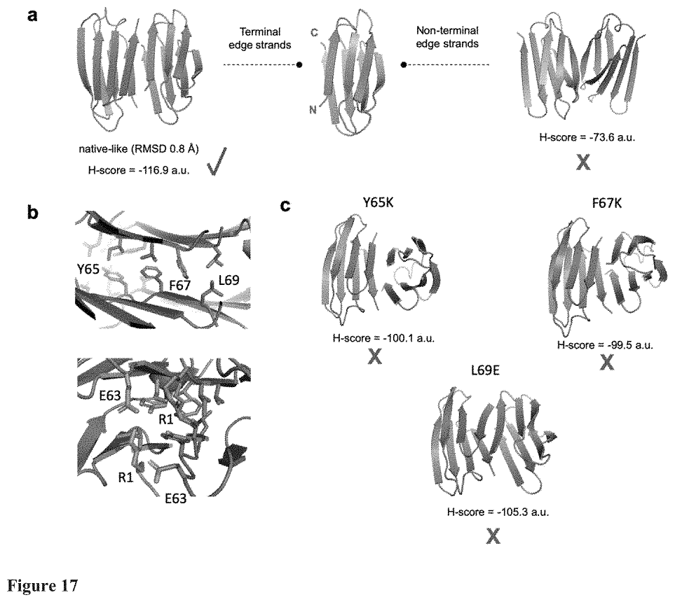

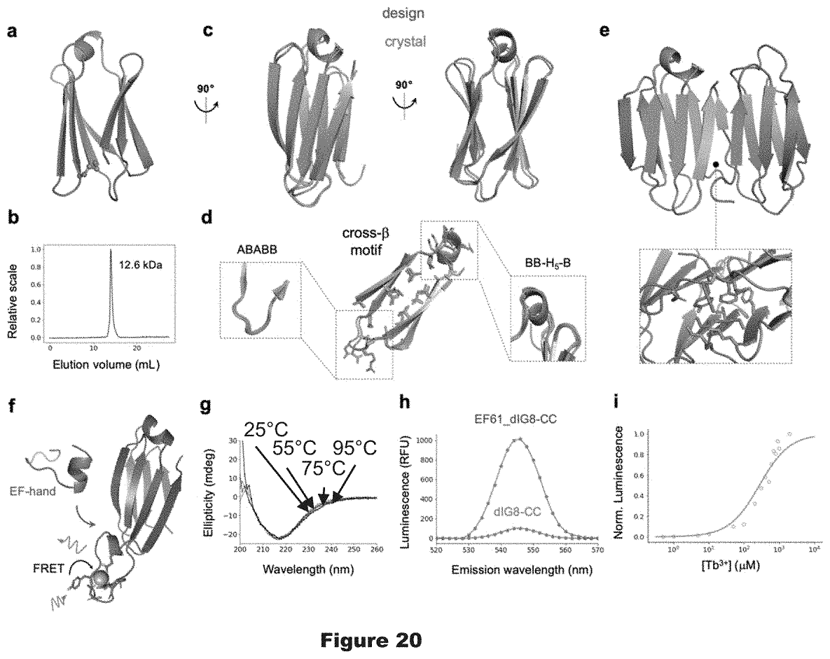

For the dIG8 design, crystallization trials yielded no hits, but we reasoned that a disulfide bond could further rigidify the structure and promote crystallization. We thus designed disulfide bonds between β-strands not forming a β-hairpin, and identified the double mutant dIG8-CC (V21C, V60C), which, like the parental protein (FIG. 12), was well-expressed, thermostable and was found in an equilibrium between monomers and dimers by SEC-MALS (FIG. 14 and FIG. 20b). We were able to obtain two crystal structures of dIG8-CC in two different space groups, with data to 2.05 and 2.30 Å resolution by molecular replacement using the design and RoseTTAFold™ predicted models (Table 8). The asymmetric unit of both crystal structures contained four protomers, and all of them closely matched the computational model with Cα-RMSDs ranging between 1.0 and 1.3 Å (FIG. 4a and FIG. 20c. The designed cross-β motif combines a β-arch loop (ABABB) with a β-arch helix (BB-H₅-B), and both were well recapitulated (Cα-RMSDs ranging between 0.7 and 1.0 Å for the two connections) across the eight monomer copies, suggesting high structural preorganization of the designed connections (FIG. 4b and FIG. 20d). The side chain of residue C21 was found in two different conformations, unbound and disulfide-bonded with C60 as in the design, which suggests that there is an equilibrium between both specimens and that the disulfide is not essential for proper folding. This is supported by the stability determined for parental dIG8. The closest Ig structural analogues found across the Protein Data Bank (PDB) and the AlphaFold Protein Structure Database (26) had a TM-score (27) ≤0.65 (FIG. 16); and contained more irregular β-strands, longer loops, and differences in the β-strand pairing organization. The crystal structures also revealed an edge-to-edge dimer interface between the N- and C-terminal β-strands, overall forming a 14-stranded β-sandwich (FIG. 4c and FIG. 20e). Docking calculations on dIG8-CC suggested that the β-sandwich edge formed by the two terminal β-strands is more dimerization-prone than the opposite edge (FIG. 17), mainly due to a more symmetrical backbone arrangement and complementary hydrophobic and salt-bridge interactions in the former, and the presence of more inward-pointing charged residues in the latter.

### Design of Functional Loops

We next sought to investigate whether de novo designed immunoglobulins could be functionalized for ligand binding. Using Rosetta™ Remodel (28), we computationally grafted and designed linkers for an EF-hand calcium-binding motif (PDB ID: 1NKF) into the three β-hairpins of dIG8-CC, and selected 12 designs for experimental testing. To assess ligand binding, we reasoned that terbium luminescence could be sensitized by energy transfer (29) by a proximal tyrosine residue on the grafted EF-hand motif (FIG. 4d and FIG. 20f). As a positive control for terbium binding, we experimentally tested a de novo designed β-barrel scaffold, EF1p2_mFAP2b (PDB ID: 6OHH), harboring an EF-hand motif with identical sequence (30). Design EF61_dIG8-CC, with the EF-hand grafted at the C-terminal β-hairpin of dIG8-CC (FIG. 4a, b and FIG. 20f) after residue 61, was the best expressed and monodisperse by SEC, thermostable by far-UV circular dichroism (FIG. 20g). EF61_dIG8-CC mixed with 100 μM TbCl₃ displayed a 10-fold higher luminescence emission intensity at 544 nm than dIG8-CC without the EF-hand motif (FIG. 20h). Tb³⁺ titrations in the presence of EF61_dIG8-CC displayed a hyperbolic increase in luminescence with increasing Tb³⁺ concentrations (FIG. 20i).

### DISCUSSION

Here, we describe the first successful de novo design of an immunoglobulin-like domain with high stability and accuracy, which was confirmed by crystal structures. This success became possible by elucidating the requirements for effective formation of cross-β motifs, which establish the non-local central core of Ig folds, by structuring β-arch connections through short loops and helices, while favoring sidechain orientations compatible with the length and pleating of the sandwiched 1-sheets.

The edge-to-edge dimer interfaces in the crystal structures of our designs differ from those found between the heavy- and light-chains of antibodies, which are arranged face-to-face, and suggest a route to de novo design rigid single-chain Ig dimers with higher structural control than single-chain variable fragments (scFvs); thereby facilitating the engineering of antibody-like formats targeting multiple epitopes. The dIG14 interface orients the N- and C-termini of the two subunits in close proximity, and a two-residue linker was predicted to correctly form the 12-stranded β-sandwich (FIG. 18). Designing Ig interfaces through the β-sandwich edge formed by the terminal β-strands also has the advantage over face-to-face dimers of decreasing the number of exposed β-strand edges, thereby reducing aggregation-propensity.

The high stability of our designs permits grafting functional loops into Ig frameworks with new backbones, as shown for the EF-hand terbium-binding loop inserted into the C-terminal β-hairpin of dIG8-CC. The present study provides a versatile generation of antibody-like scaffolds with improved properties.

TABLE 6
Cross-β geometrical parameters calculated for the designed
proteins. For comparison, median and median absolute deviation
values for cross-β parameters calculated from naturally
occurring Ig domain structures are also provided, as shown in FIG. 8a.

| Design | Distance (\acute{Å}) | Twist (°) | Roll (°) | Tilt (°) |
|---|---|---|---|---|
| dIG1 | 11.5 | −3.4 | 12.6 | −6.4 |
| dIG2 | 10.7 | −9.2 | 14.6 | 6.8 |
| dIG3 | 11.3 | −12.4 | 20.5 | 27.1 |
| dIG4 | 11.0 | −10.8 | 10.6 | 7.2 |
| dIG5 | 10.2 | −24.7 | −5.7 | −8.3 |
| dIG6 | 11.2 | 5.5 | 4.3 | −4.8 |
| dIG7 | 10.4 | −18.7 | −7.4 | −18.8 |
| dIG8 | 10.0 | −16.6 | 6.8 | 3.1 |
| dIG9 | 10.3 | −13.9 | 11.6 | 8.3 |
| dIG10 | 11.0 | −8.6 | 6.6 | −2.7 |
| dIG11 | 11.1 | −17.1 | 5.2 | 8.1 |
| dIG12 | 10.7 | 2.0 | 11.0 | 4.0 |
| dIG13 | 11.46 | −19.2 | −4.7 | −10.4 |
| dIG14 | 11.2 | −13.8 | 9.9 | 4.4 |
| dIG15 | 11.0 | −16.8 | 11.3 | −5.1 |
| dIG16 | 10.5 | −14.5 | −4.1 | −12.5 |
| dIG17 | 9.8 | −2.6 | −4.9 | −0.3 |
| dIG18 | 12.2 | −1.8 | −0.1 | −2.0 |
| dIG19 | 11.2 | −5.7 | 19.2 | 1.8 |
| dIG20 | 10.8 | 11.9 | −9.8 | −2.9 |
| dIG21 | 10.9 | −18.4 | −17.6 | −19.0 |
| dIG22 | 10.7 | 4.5 | 9.9 | −5.8 |
| dIG23 | 10.8 | 0.3 | 3.2 | −1.1 |
| dIG24 | 10.3 | −20.7 | −8.8 | −12.5 |
| dIG25 | 10.0 | −21.5 | −0.7 | −17.4 |
| dIG26 | 12.2 | 0.6 | 20.0 | 16.1 |
| dIG27 | 10.8 | −15.7 | 6.1 | 10.7 |
| dIG28 | 10.4 | 1.2 | 5.5 | 1.3 |
| dIG29 | 11.0 | −8.6 | 6.7 | 2.7 |
| dIG30 | 10.8 | −18.2 | 6.5 | −7.1 |
| dIG31 | 11.9 | −7.9 | 13.5 | 26.5 |
| Natural Ig | 10.9 ± 0.8 | −32.1 ± 7.7 | 12.0 ± 12.2 | 4.0 ± 11.1 |
| domains | | | | |

TABLE 7
Summary of the experimental characterization of designs.

| | Soluble | | CD spectra | | Oligomeric |
|---|---|---|---|---|---|
| dIG | expression | Monodisperse | (25° C.) | Tₘ(° C.) | state† |
| 1 | No | | | | |
| 2-6, 9, 11-13, | Yes | No | | | |
| 16-19, 24-31 | | | | | |
| 10, 20 | Yes | Yes | β | >95° C. | High |
| 21 | Yes | Yes | β | >75° C. | D |
| 7, 14, 15, 22, 23 | Yes | Yes | β | >95° C. | D |
| 8,8-CC | Yes | Yes | β | >95° C. | M/D |

†Oligometric state of the dominant species determined with size-exclusion chromatography coupled with multi-angle light-scattering SEC-MALS (‘M’, monomer; ‘D’, dimer).

TABLE 8
Crystallographic data.

| Dataset | dIG8-CC (tetragonal) | | dIG8-CC (orthorhombic) | | dIG14 | |
|---|---|---|---|---|---|---|
| Beam line (synchrotron) | ID30A-3 (ESRF) | | XALOC (ALBA) | | XALOC (ALBA) | |
| Space group/complexes per a.u. ª | P4₁2₁2/4 | | C222₁/4 | | P4₃2₁2/2 | |
| Cell constants (a, b and c in Å) | 58.17, 58.17, 173.50 | | 43.03, 76.52, 165.80 | | 73.91, 73.91, 97.48 | |
| Wavelength (Å) | 0.96770 | | 0.97879 | | 0.97918 | |
| Measurements/unique reflections | 409,652/13,994 | | 221,806/17,590 | | 481,439/9805 | |
| Resolution range (Å) (outermost shell) ^{c} | 55.2-2.30 | (2.43-2.30) | 38.3-2.05 | (2.17-2.05) | 58.9-2.50 | (2.65-2.50) |
| Completeness (%)/R_{merge} ^{d} | 99.9 (99.7)/0.178 (2.829) | | 99.6 (98.0)/0.101 (2.453) | | 99.6 (99.3)/0.096 (3.057) | |
| Rₚᵢₘ ^{e}/CC(½) ^{e} | 0.033 (0.520)/0.999 (0.865) | | 0.030 (0.781)/1.000 (0.601) | | 0.014 (0.435)/0.999 (0.901) | |
| Average intensity ^{f} | 15.2 | (1.5) | 15.2 | (1.3) | 30.2 | (3.2) |
| B-Factor (Wilson) (Å²)/Aver. multiplicity | 58.4/29.3 | (30.6) | 56.2/12.6 | (10.9) | 86.2/49.1 | (50.4) |
| Resolution range used for refinement (Å) | 37.2-2.30 | | 38.3-2.05 | | 58.9-2.50 | |
| Reflections used (test set) | 13,415 | (511) | 16,855 | (706) | 9429 | (376) |
| Crystallographic R_{factor} (free R_{factor}) ^{d} | 0.263 | (0.301) | 0.250 | (0.291) | 0.247 | (0.300) |
| Non-H protein atoms/waters/ligands per a.u. | 2316/22/— | | 2138/34/1 | Mg²⁺ | 1190/15/— | |
| Rmsd from target values | | | | | | |
| bonds (Å)/angles (°) | 0.002/0.48 | | 0.002/0.43 | | 0.008/0.87 | |
| Average B-factor (Å²) | 63.7 | | 63.2 | | 94.6 | |
| Protein contacts and geometry analysis ^{b} | | | | | | |
| Ramachandran favoured/outliers/all analysed | 284 (98.3%)/1/289 | | 279 (100%)/0/279 | | 142 (100%)/0/142 | |
| Bond-length/bond-angle/chirality/planarity outliers | 0/0/0/0 | | 0/0/0/0 | | 0/0/0/0 | |
| Sidechain outliers | 8 | (3.1%) | 2 | (0.9%) | 8 | (6.0%) |

| | | | |
|---|---|---|---|
| All-atom clashes/clashscore ^{b} | 18/3.9 | 16/3.8 | 11/4.7 |
| RSRZ outliers/Fₒ:F_{c} correlation | 16 (5.5%) ^{b}/0.93 | 15 (5.3%) ^{b}/0.95 | 12 (8.5%) ^{g}/0.93 |
| PDB access code | 7SKN | 7SKO | 7SKP |

^{a} Abbreviations: EDO, ethylene glycol; PEG, diethylene glycol; RSRZ, real-space R-value Z-score.
^{b} According to the wwPDB Validation Service.
^{c} Values in parenthesis refer to the outermost resolution shell if not otherwise indicated.
^{d} For definitions, see Table 1 in(2).
^{e} For definitions, see (3, 4).
^{f} Average intensity is <I/σ(I)> of unique reflections after merging according to Xscale (5).
^{g} According to Coot (<0.15; (6)).

### REFERENCES

1. C. Jost, A. Plückthun, Engineered proteins with desired specificity: DARPins, other alternative scaffolds and bispecific IgGs. Curr Opin Struct Biol. 27, 102-112 (2014).
2. J. R. Kintzing, M. V. Filsinger Interrante, J. R. Cochran, Emerging Strategies for Developing Next-Generation Protein Therapeutics for Cancer Treatment. Trends Pharmacol Sci. 37, 993-1008 (2016).
3. F. Sha, G. Salzman, A. Gupta, S. Koide, Monobodies and other synthetic binding proteins for expanding protein science: Monobodies and Other Synthetic Binding Proteins. Protein Sci. 26, 910-924 (2017).
4. E. Marcos, D. Silva, Essentials of de novo protein design: Methods and applications. WIREs Comput Mol Sci. 8 (2018), doi:10.1002/wcmos.1374.
5. E. Marcos et al., Principles for designing proteins with cavities formed by curved β sheets. Science. 355, 201-206 (2017).
6. J. Dou et al., De novo design of a fluorescence-activating β-barrel. Nature. 561, 485-491 (2018).
7. N. Koga et al., Principles for designing ideal protein structures. Nature. 491, 222-227 (2012).
8. E. Marcos et al., De novo design of a non-local β-sheet protein with high stability and accuracy. Nat Struct Mol Biol. 25, 1028-1034 (2018).
9. A. A. Vorobieva et al., De novo design of transmembrane β barrels. Science. 371 (2021), doi:10.1126/science.abc8182.
10. P. Bork, L. Holm, C. Sander, The Immunoglobulin Fold. J Mol Biol. 242, 309-320 (1994).
11. D. M. Halaby, A. Poupon, J.-P. Mornon, The immunoglobulin fold family: sequence analysis and 3D structure comparisons. Protein Engineering, Design and Selection. 12, 563-571 (1999).
12. J. Hennetin, B. Jullian, A. C. Steven, A. V. Kajava, Standard Conformations of β-Arches in β-Solenoid Proteins. J Mol Biol. 358, 1094-1105 (2006).
13. A. E. Kister, A. V. Finkelstein, I. M. Gelfand, Common features in structures and sequences of sandwich-like proteins. Proc Natl Acad Sci USA. 99, 14137-14141 (2002).
14. K. W. Plaxco, K. T. Simons, D. Baker, Contact order, transition state placement and the refolding rates of single domain proteins. J Mol Biol. 277, 985-994 (1998).
15. J. K. Leman et al., Macromolecular modeling and design in Rosetta: recent methods and frameworks. Nat Methods. 17, 665-680 (2020).
16. Y.-R. Lin et al., Control over overall shape and size in de novo designed proteins. Proc Natl Acad Sci USA. 112, E5478-E5485 (2015).
17. B. Kuhlman, D. Baker, Native protein sequences are close to optimal for their structures. Proc Natl Acad Sci USA. 97, 10383-10388 (2000).
18. B. Kuhlman et al., Design of a Novel Globular Protein Fold with Atomic-Level Accuracy. Science. 302, 1364-1368 (2003).
19. J. S. Richardson, D. C. Richardson, Natural β-sheet proteins use negative design to avoid edge-to-edge aggregation. Proc Natl Acad Sci USA. 99, 2754-2759 (2002).
20. P. Bradley, Toward High-Resolution de Novo Structure Prediction for Small Proteins. Science. 309, 1868-1871 (2005).
21. J. Jumper et al., Highly accurate protein structure prediction with AlphaFold. Nature. 596, 583-589 (2021).
22. M. Baek et al., Accurate prediction of protein structures and interactions using a three-track neural network. Science. 373, 871-876 (2021).
23. C. Camacho et al., BLAST+: architecture and applications. BMC Bioinformatics. 10, 421 (2009).
24. M. Remmert, A. Biegert, A. Hauser, J. Söding, HHblits: lightning-fast iterative protein sequence searching by HMM-HMM alignment. Nat Methods. 9, 173-175 (2012).
25. L. Zimmermann et al., A Completely Reimplemented MPI Bioinformatics Toolkit with a New HHpred Server at its Core. J Mol Biol. 430, 2237-2243 (2018).
26. K. Tunyasuvunakool et al., Highly accurate protein structure prediction for the human proteome. Nature. 596, 590-596 (2021).
27. Y. Zhang, TM-align: a protein structure alignment algorithm based on the TM-score. Nucleic Acids Res. 33, 2302-2309 (2005).
28. P.-S. Huang et al., RosettaRemodel: A Generalized Framework for Flexible Backbone Protein Design. PLoS ONE. 6, e24109 (2011).
29. S. C. Zondlo, F. Gao, N. J. Zondlo, Design of an Encodable Tyrosine Kinase-Inducible Domain: Detection of Tyrosine Kinase Activity by Terbium Luminescence. J Am Chem Soc. 132, 5619-5621 (2010).
30. J. C. Klima et al., Incorporation of sensing modalities into de novo designed fluorescence-activating proteins. Nat Commun. 12, 856 (2021).
31. T. P. Quinn et al., Betadoublet: de novo design, synthesis, and characterization of a beta-sandwich protein. Proc Natl Acad Sci USA. 91, 8747-8751 (1994).
32. Y. Yan, B. W. Erickson, Engineering of betabellin 14D: Disulfide-induced folding of a β-sheet protein. Protein Sci. 3, 1069-1073 (1994).
33. M. H. Hecht, De novo design of beta-sheet proteins. Proc Natl Acad Sci USA. 91, 8729-8730 (1994).

### Methods

### Structural Analysis of β-Arch Loops

β-arch loops of less than 9 residues were collected from a non-redundant set of 5,857 PDB structures with sequence identity <30% and resolution ≤2.0 Å. They were identified by first assigning the secondary structure with DSSP (34), and ensuring they were connecting β-strands with no hydrogen-bond pairing between them. The ABEGO torsion bins of each loop position were assigned based on their φ/ψ backbone dihedrals as defined in FIG. 6a. The sidechain orientations of the two residues (i and j) preceding and following the β-arch loop are a function of the relative orientation between their C_{α}C_{β} vector and the translation vector (v₁) connecting their C_{α}'s, as shown in FIG. 6b. The β-arch sliding distance was calculated as the dot product between v₁ and the CO vector of the preceding residue (v₁·COᵢ), which points along the β-sheet hydrogen bond direction. If the dot product between v₁ and the C_{α}C_{β}(i) vector of the preceding residue is negative, then the sliding distance is calculated as v₁·−COᵢ. The β-arch twist was calculated as the dihedral between positions C_{α}(i−2), C_{α}(i), C_{α}(j), and C_{α}(j+2).

### Cross-β Motif Analysis

To extract the cross-β geometrical parameters, we calculated the rigid body transformation between two reference frames defined at the two β-sheets comprising the cross-β motif. The reference frames were built with the vectors described above for verifying cross-β formation, i.e. S₁, S₃₁ and P_{N} for the first β-sheet; and S₄, S₂₄ and P_{C} for the second β-sheet. To minimize the dependence of cross-β parameters on differences in the internal geometry of β-strands from the two different β-sheets, we pre-generated a template antiparallel strand dimer that, before calculating the transform, is superimposed on each of the two strand dimers of the cross-β motif. The transform rotational angles were calculated as the Euler angles of the transform (twist, roll and tilt). The cross-β motif distance was calculated between the centers of the two strand dimers. The β-arch sliding distance in a cross-β motif was calculated as the dot product between the translation vectors and the vector S₃₁ connecting the centers of the two N-terminal strands (1 and 3), as defined in FIG. 7.

### Structural Analysis of Naturally Occurring Immunoglobulin-Like Domains

We searched for Ig-like domains classified in SCOP (35) as “Ig-like beta-sandwich” folds (SCOP ID 2000051) and selected those with X-ray resolution ≤2.5 Å, yielding a total of 467 annotated domains.

### Protein Backbone Generation and Sequence Design

We specified blueprint files for each target protein topology and constructed poly-valine backbones with the RosettaScripts™ (36) implementation of the BluePrintBDR (7) mover, which carries out Monte Carlo fragment assembly using 9- and 3-residue fragments picked based on the secondary structure and ABEGO torsion bins specified at each residue position. We used the fldsgn_cen centroid scoring function with reweighted terms accounting for backbone hydrogen bonding (lr_hb_bb) and planarity of the peptide bond (omega).

For constructing cross-β motifs, we followed a two-step procedure. First, the two N-terminal strands of the motif (strands 1 and 3) were generated as antiparallel β-strand dimers of desired length from φ/ψ values typical of β-strands (extended region of the Ramachandran plot) and relaxed using hydrogen-bond pairing restraints. Second, the cross-β loops and C-terminal strands (strands 2 and 4) were then appended by fragment assembly using the BluePrintBDR, as described above, combined with a strand pairing energy bonus between strands 2 and 4. We assign the two N-terminal strands to different chains (A and B), and the resulting jump between the two chains allows to fold the two C-terminal strands independent of each other. Then, the secondary structures of the resulting backbones were calculated by DSSP (34) and those with a secondary structure identity to that defined in the blueprints below 90% were discarded to guarantee correct strand pairing formation. The filtered backbones needed to fulfill two additional properties to be considered a cross-β motif: (1) the two C-terminal strands must form antiparallel strand pairing with each other, but not with any of the N-terminal strands (to guarantee β-sandwich formation); (2) the two β-arches must cross. For the latter, we checked crossing based on the relative orientation between the two vectors orthogonal to each of the two β-sheet planes packing face-to-face. The P_{N} vector orthogonal to the β-sheet formed by the two N-terminal strands is calculated as the cross product between the S₁ and S₃₁ vectors (P_{N}=S₁×S₃₁); where S₁ defines the direction of β-strand 1 (from N to C-termini) and S₃₁ connects the centers of the two N-terminal strands (1 and 3). The P_{C} vector orthogonal to the β-sheet formed by C-terminal strands is calculated similarly as P_{C}=S₄×S₂₄. If the two orthogonal vectors are parallel (if P_{N}·P_{C}>0) the two β-arches were considered to cross.

For designing 7-stranded Ig backbones, we carried out hundreds of independent blueprint-based trajectories folding each target topology in one step followed with a backbone relaxation using strand pairing constraints. We encouraged correct formation of strand pairs using custom python scripts writing distance and angle constraints specifying backbone hydrogen bond pairing at each pair of residue positions. The generated backbones were subsequently filtered based on their match with the secondary structure and ABEGO torsion bins specified in the corresponding blueprint files, and their long-range backbone hydrogen bond energy (lr_hb_bb score term). We carried out FastDesign (37) calculations using the Rosetta™ all-atom energy function ref2015 (38) to optimize sidechain identities and conformations with low-energy, efficiently packing the protein core, and compatible with their solvent accessibility. Designed sequences were filtered based on the average total energy, Holes score (39), buried hydrophobic surface, and sidechain-backbone hydrogen bond energy (for better stabilizing β-arch geometry). For loop residue positions we restricted amino acid identities based on sequence profiles derived from naturally occurring loops with the same ABEGO torsion bins (5).

### Sequence-Structure Compatibility Evaluation

The local compatibility between the designed sequences and structures was evaluated based on fragment quality. Sequence-structure pairs were considered locally compatible if for all residue positions at least one of the picked 9-mer fragments (based on sequence and secondary structure similarity with the design) had a RMSD below 1.0 Å. For designs fulfilling this requirement, we assessed their folding by Rosetta™ ab initio structure prediction in two steps. We started screening hundreds of designs quickly with biased forward folding simulations (5) (BFF) using the three 9- and 3-mers closer in RMSD to the design. Those designs with a substantial fraction (>10%) of BFF trajectories sampling structures with RMSDs to the design below 1.5 Å were then selected for standard Rosetta™ ab initio structure prediction (20). We ran AlphaFold™ (21) and the PyRosetta™ version of RoseTTAFold™ (22) with a local installation and using default parameters.

### Docking Calculations

HADDOCK™ (40) was used for the evaluation of the crystallographic interface of the design. We picked the first chain from the dIG8-CC crystal structure and used two copies of this monomer for all two-body docking simulations. Taking advantage of the ability of HADDOCK™ to build missing atoms, we constructed the mutants by renaming and removing all atoms but those forming the backbone (N, C_{α}, C, O) and the C_{β} (to maintain sidechain directionality). For the simulations targeting the crystallographic interface, we selected all residues pertaining to the first and seventh strands (segments 1-7 and 65-70) as active residues to drive the docking. For the ones aiming to the opposite interface, all residues from the third and fourth strands (segments 30-35 and 39-45) were instead used as active residues. For all docking simulations, we defined two different sets of symmetry restraints as follows: (1) We applied C2 symmetry restraints to assure a 180° symmetry axis between both molecules and (2) enabled non-crystallographic restraints (NCS) to enforce identical intermolecular contacts. All remaining docking and analysis parameters were kept as default. In terms of analysis, the generated models were evaluated by the default HADDOCK™ scoring function. This mathematical approximation is a weighted linear combination of different energy terms including: van der Waals and electrostatic intermolecular energies, a desolvation potential and a distance restraint energy term. The scoring step is followed by a clustering procedure based on the fraction of common contacts, and the resulting clusters are re-ranked according to the average HADDOCK™ score of the best 4 cluster members. For comparison purposes, we used the exact same set of parameters for all docking simulations and selected the top model from the best ranked cluster.

### Design of Disulfide Bonds

The identification of the position of disulfide bonds was carried out with a novel motif hashing protocol (41). 30,000 examples of native disulfide geometries were extracted from high-resolution protein crystal structures of the PDB. The relative orientation of the backbone atoms was calculated by determining the translation and rotation matrix between the two sets of backbone atoms. These translation and rotation matrices were hashed and stored in a hash table with the associated conformation of the sidechains. Once the hash table has been completed by including all of the examples of disulfides from the PDB, the hash table can be utilized to place disulfides into de novo designed proteins by evaluating the relative orientation within a designed protein to find which residue pairs match an example from the hash table.

### Design of EF-Hand Calcium Binding Motifs

A minimal EF-hand motif from Protein Data Bank (PDB) accession code 1NKF (42) was generated by truncating the PDB file 3-dimensional coordinates to the minimal Ca²⁺-binding sequence DKDGDGYISAAE (SEQ ID NO:303). RosettaRemodel™ (28) blueprint files were generated from the 3-dimensional coordinates of the dIG8 computational model and minimal EF-hand motif, and an in-house script used to write RosettaRemodel™ blueprint files for domain insertion of the minimal EF-hand motif into dIG8. 132 blueprint files were generated to insert the EF-hand motif after residues 8, 28, and 61 of dIG8 while systematically sampling N-terminal linker lengths of 0-3 residues with β-sheet secondary structure and C-terminal linker lengths of 0-10 residues with α-helical secondary structure. RosettaRemodel™ was run three times for each blueprint file using the pyrosetta.distributed and dask python modules (43-45). Linker compositions were de novo designed in RosettaRemodel™ using specific sets of amino acids defined in the blueprint files at each position of the N-terminal and C-terminal linkers while preventing repacking of EF-hand motif sidechain rotamers required for chelating Ca²⁺. Out of 396 domain insertion simulations, 86 successfully closed the N-terminal and C-terminal linkers producing single-chain decoys. On each decoy, a custom PyRosetta™ script was run to append a Ca²⁺ ion into the EF-hand motif. Decoys were then relaxed via Monte Carlo sampling of protein sidechain repacking and protein sidechain and backbone minimization steps with a full-atom Cartesian coordinate energy function (38) with coordinate constraints applied to the aspartate and glutamate residues chelating the Ca²⁺ ion. The 86 resulting designs were scored in RosettaScripts™ (36) with an in-house XML script. Concomitantly, each of the 86 designs were forward folded (20) after temporarily stripping out the Ca²⁺ ion from each decoy, and the ff_metric algorithm used to evaluate funnels (46). To select designs for experimental validation, the following computational protein design metric filters were applied: buns_all_heavy_ball ≤1.0; buns_all_heavy_ball_interface ≤1.0: total_score_res ≤−3.7; geometry=1.0. Filtered designs were ranked ascending primarily on buns_all_heavy_ball, ascending secondarily on ff_metric, and ascending tertiarily on total_score_res. To experimentally test designs at the three domain insertion sites, the top three ranked designs at each of the three domain insertion sites were selected. To experimentally test designs with the shortest N-terminal and C-terminal linkers, the top three ranked designs with up to a 3-residue N-terminal linker and up to a 2-residue C-terminal linker were selected. 12 designs in total were selected for experimental characterization after mutating positions compatible with disulfide bonds to cysteines.

### Recombinant Expression and Purification of the Designed Proteins for Biophysical Studies

Synthetic genes encoding for the selected amino acid sequences were ordered from Genscript and cloned into the pET-28b+ expression vector, with the genes of interest inserted within NdeI and XhoI restriction sites and the pET28b backbone encoding an N-terminal, thrombin-cleavable His6-tag. Escherichia coli BL21 (DE3) competent cells were transformed with these plasmids, and starter cultures from single colonies were grown overnight at 37° C. in Luria-Bertani (LB) medium supplemented with kanamycin. Overnight cultures were used to inoculate 50 ml of Studier autoinduction media (47) with antibiotic as done in a previous study (48). Cells were harvested by centrifugation and resuspended in a 25 mL lysis buffer (20 mM imidazole in PBS containing protease inhibitors), and lysed by microfluidizer. PBS buffer contained 20 mM NaPO₄, 150 mM NaCl, pH 7.4. After removal of insoluble pellets, the lysates were loaded onto nickel affinity gravity columns to purify the designed proteins by immobilized metal-affinity chromatography (IMAC). The expression of purified proteins was assessed by SDS-polyacrylamide gel; and protein concentrations were estimated from the absorbance at 280 nm measured on a NanoDrop™ spectrophotometer (ThermoScientific) with extinction coefficients predicted from the amino acid sequences using the ProtParam tool. Proteins were further purified by size-exclusion chromatography using a Superdex™ 75 10/300 GL (GE Healthcare) column.

### Circular Dichroism

Far-UV circular dichroism measurements were carried out with a JASCO™ spectrometer. Wavelength scans were measured from 260 to 195 nm at temperatures between 25 and 95° C. with a 1 mm path-length cuvette. Protein samples were prepared in PBS buffer (pH 7.4) at a concentration of 0.3-0.4 mg/mL. GdnCl solutions were prepared by dissolving GdnCl salt into PBS buffer and checking the refractive index.

### Size-Exclusion Chromatography Coupled to Multiple-Angle Light Scattering (SEC-MALS)

To ascertain the oligomerisation state of dIG proteins, SEC-MALS was performed in a Dawn Helios™ II apparatus (Wyatt Technologies) coupled to a SEC Superdex™ 75 Increase 10/300 column. The column was equilibrated with PBS or buffer B at 25° C. and operated at a flow rate of 0.5 mL/min. A total volume of 100-165 μL of protein solution at 1-3.0 mg/mL was employed for each sample. Data processing and analysis proceeded with Astra 7 software (Wyatt Technologies), for which a typical dn/dc value for proteins (0.185 mL/g) was assumed.

### Protein Production for Crystallization Studies

The original thrombin site of plasmids pET28-dIG8-CC and pET28-dIG14 was replaced with a Tobacco-Etch-Virus peptidase (TEV) recognition site via NcoI and Nde employing forward and reverse primers (Eurofins). The generated plasmids, pET28*-dIG8-CC and pET28*-dIG14, were mixed at 100 mg each in Takara buffer (50 mM Tris-HCl, 10 mM magnesium chloride, 1 mM dithiothreitol, 100 mM sodium chloride, pH 7.5), annealed by slowly cooling down the sample to room temperature following 4 minutes at 94° C., and ligated into the doubly digested plasmid. For pET28*-dIG14, the original thrombin-cleavable N-terminal His₆-tag was removed and four histidine residues were added to the protein C-terminus by PCR using NcoI and XhoI sites. Of note, due to the cloning strategy, dIG18-CC and dIG-14 proteins were preceded by a G-H-M and a M-G motif, respectively. All PCR reactions and ligations were performed using Phusion™ High Fidelity DNA polymerase and T4 Ligase, and ligation products were transformed into chemically competent E. coli DH5-α, cells for multiplication (all Thermo Fisher Scientific). Plasmids were purified with the E.Z.N.A.™ Plasmid Mini Kit I (Omega Bio-Tek) and verified by sequencing (Eurofins and Macrogen).

For protein expression, competent E. coli BL21 (DE3) cells (Sigma) were transformed with the pET28*-dIG8-CC and pET28*-dIG14 plasmids and grown on LB plates supplemented with 100 μg/mL kanamycin. Single colonies were selected to inoculate 5-mL starter cultures of this medium and incubated overnight at 37° C. under shaking. Respective 1-mL aliquots were used to inoculate 500 mL of the same medium. Once cultures reached OD₆₀₀≈0.6, protein expression was induced with 0.5 mM IPTG (Fisher Bioreagents), and cultures were incubated overnight at 18° C. Cells were harvested by centrifugation (3,500×g, 30 min, 4° C.) and resuspended in cold buffer A (50 mM Tris·HCl, 250 mM sodium chloride, pH 7.5), supplemented with 10 mM imidazole, EDTA-free cOmplete™ Protease Inhibitor Cocktail (Roche Life Sciences), and DNase I (Roche Life Sciences). Cells were lysed using a cell disrupter (Constant Systems) operated at 135 MPa, and soluble protein was clarified by centrifugation (50,000×g, 1 h, 4° C.) and subsequently passed through a 0.22-μm filter (Merck Millipore).

For immobilized metal-affinity chromatography (IMAC (49)), proteins were captured on nickel-sepharose HisTrap™ HP columns (Cytiva), which had previously been washed and pre-equilibrated with buffer A plus either 500 mM or 20 mM imidazole, respectively. Column-bound dIG14 was extensively washed with a gradient of 20-to-150 mM imidazole in buffer A and eluted with a gradient of 200-to-300 mM imidazole in buffer A. Column-bound dIG8-CC was washed and eluted with buffer A containing 20 mM and 300 mM imidazole, respectively.

Fractions containing the dIG8-CC protein were then buffer-exchanged to buffer B (20 mM Tris·HCl, 150 mM sodium chloride, pH 7.5) in a HiPrep™ 26/10 desalting column (GE Healthcare), and incubated overnight at 4° C. with inhouse-produced His₆-tagged TEV peptidase at a peptidase:substrate ratio of 1:20 (w/w) for fusion-tag removal. After centrifugation (50,000×g. 1 h, 4° C.) and filtration (0.22-μm), the clarified dIG8-CC protein was loaded again onto the HisTrap HP column for reverse IMAC with buffer A plus 20 mM imidazole, which retained tagged protein and TEV, and had untagged dIG8-CC in the flow-through. The bound proteins were eventually eluted with buffer A plus 300 mM imidazole for column regeneration.

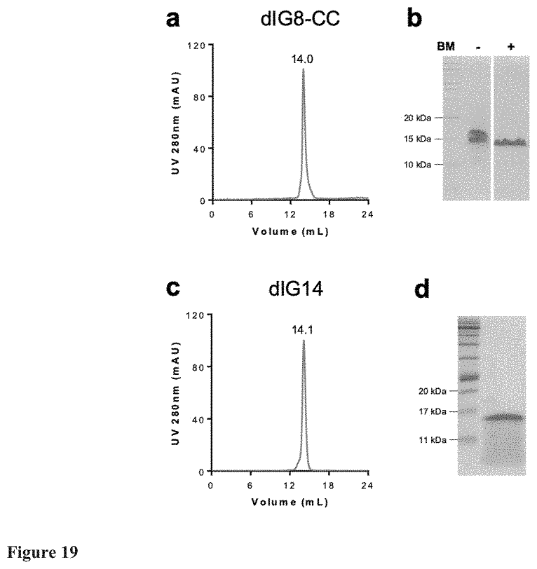

Untagged dIG8-CC and dIG14 were polished by size-exclusion chromatography (SEC) with buffer B in a Superdex™ 75 Increase 10/300 GL column (Cytiva) attached to an ÄKTA™ Purifier 10 apparatus. Protein purity was assessed by 20% SDS-PAGE stained with Coomassie Brilliant Blue (Sigma). PageRule™ Unstained Broad Range Protein Ladder and PageRuler™ Plus Prestained Protein Ladder (both Thermo Fisher Scientific) were used as molecular-mass markers. To concentrate protein samples, ultrafiltration was performed using Vivaspin 15 and Vivaspin 2 Hydrosart™ devices (Sartorius Stedim Biotech) of 2-kDa molecular-mass cutoff. Protein concentrations were determined either by the BCA Protein Assay Kit (Thermo Fisher Scientific) with bovine serum albumin as a standard or by A₂₈₀ using a BioDrop™ Duo+ apparatus (Biochrom). FIG. 19 provides proof of the effective protein purification procedures.

### Protein Crystallization

Crystallization screenings using the sitting-drop vapor diffusion method were performed at the joint IRB/IBMB Automated Crystallography Platform at Barcelona Science Park (Catalonia, Spain). Screening solutions were prepared and dispensed into the reservoir wells of 96×2-well MRC crystallization plates (Innovadyne Technologies) by a Freedom EVO™ robot (Tecan). These reservoir solutions were employed to pipet crystallization nanodrops of 100 nL each of reservoir and protein solution into the shallow crystallization wells of the plates, which were subsequently incubated in steady-temperature crystal farms (Bruker) at 4° C. or 20° C.

After refinement of initial hit conditions, suitable dIG14 crystals appeared at 20° C. in drops consisting of 0.5 μL protein solution (at 1.9 mg/mL in buffer B) and 0.5 μL reservoir solution (0.1 M sodium acetate, 0.2 M calcium chloride, 20% w/v polyethylene glycol [PEG]1500, pH 5.5). Crystals were cryoprotected with reservoir solution supplemented with 20% glycerol, harvested using 0.1-0.2 mm nylon loops (Hampton), and flash-vitrified in liquid nitrogen. The best tetragonal dIG8-CC crystals were obtained at 20° C. in drops containing 0.5 μL protein solution (at 30 mg/mL in buffer B) and 0.5 μL reservoir solution (0.1 M Bis-Tris, 0.2 M calcium chloride, 20% w/v PEG 3350, 10% v/v ethylene glycol, pH 6.5). Crystals were directly harvested using 0.1-0.2 mm loops, and flash-vitrified in liquid nitrogen. Proper orthorhombic dIG8-CC crystals resulted from the same condition as the tetragonal ones except that magnesium chloride and glycerol replaced calcium chloride and ethylene glycol, respectively. Furthermore, 0.25 mL of 5% n-dodecyl-N,N-dimethylamine-N-oxide (w/v) was included as an additive. These crystals were cryoprotected with reservoir solution supplemented with 20% glycerol, harvested with elliptical 0.02-0.2 mm LithoLoops™ (Molecular Dimensions), and flash-vitrified in liquid nitrogen.

### Diffraction Data Collection and Structure Solution

X-ray diffraction data were recorded at 100 K on a Pilatus™ 6M pixel detector (Dectris) at the XALOC beamline (50) of the ALBA synchrotron (Cerdanyola, Catalonia, Spain) and on an EIGER™ X 4M detector (Dectris) at the ID30A-3 beamline (51) of the ESRF™ synchrotron (Grenoble, France). Diffraction data were processed with programs Xds (52) and Xscale, and transformed with Xdsconv to MTZ-format for the Phenix (53) and CCP4 (54) suites of programs. Analysis of the data with Xtriage (55) within Phenix and Pointless (56) within CCP4 confirmed the respective space groups and indicated absence of twinning and translational non-crystallographic symmetry. Table 10 provides essential statistics on data collection and processing.

The structure of dIG8-CC, both in its tetragonal (P4₁2₁2; 2.30 Å) and orthorhombic (C222₁; 2.05 Å) space groups, was solved by molecular replacement with the Phaser (57) program employing the coordinates of the designed structure. The tetragonal crystals contained four protomers (chains A-D) in the asymmetric unit (a.u.) arranged as two dimers, and the calculations gave final refined values of the translation function Z-score (TFZ) and log-likelihood gain (LLG) of 14.5 and 307, respectively. Subsequently, the adequately rotated and translated molecules were subjected to successive rounds of manual model building with the Coot program (58) alternating with crystallographic refinement with the Refine protocol of Phenix (59), which included translation/libration/screw-motion (TLS) refinement and non-crystallographic symmetry (NCS) restraints. The final model included residues R¹-G⁷⁰ of each protomer preceded by M⁰, H⁻¹, and, in chain D only, G⁻² from the upstream linker, as well as 22 solvent molecules. The orthorhombic crystals were solved as the tetragonal ones with final refined TFZ and LLG values of 11.9 and 263, respectively. Model building and refinement proceeded as above. The final model encompassed residues R¹-G⁷⁰ of each protomer preceded by M⁰ and H⁻¹, plus one magnesium cation and 34 solvent molecules. Unexpectedly, cysteines C²¹ and C⁶⁰ were present in both disulfide-linked and unbound conformations in all protomers of both crystal forms.

The structure of dIG14 in a yet different space group (P4₃2₁2; 2.50 Å) with two molecules per a.u. was likewise solved by molecular replacement, with final refined TFZ and LLG values amounting to 17.4 and 269, respectively. The phases derived from the adequately rotated and translated molecules were subjected to a density modification and automatic model building step under twofold averaging with the Autobuild routine of Phenix, which produced a Fourier map that assisted model building as aforementioned. Crystallographic refinement was also performed as above except that both Phenix and the BUSTER package (61) were employed. The final model comprised R¹-G⁶⁸ of protomer A and R¹-F⁷⁴ of protomer B, either preceded by G⁰ and M⁻¹ from the upstream linker, as well as 15 solvent molecules. Table 9 provides essential statistics on the final refined models, which were validated through the wwPDB Validation Service.

### Tb³⁺ Luminescence Assay

Designs dIG8-CC and EF61_dIG8-CC were expressed and purified by IMAC and size-exclusion chromatography (SEC) in phosphate-buffered saline (PBS; 25.0 mM phosphate, 150 mM NaCl, pH 7.40). Control proteins EF1p2_mFAP2b and mFAP2b were expressed and purified by IMAC as described previously (62) by large-scale protein purification in low salt Tev cleavage buffer (20.0 mM Tris, 50.0 mM NaCl, pH 7.40). Protein concentrations were measured with a QuBit™ 2.0 fluorimeter (ThermoFisher Scientific, Q32866) and QuBit™ Protein Assay Kit (ThermoFisher Scientific, Q33212), and protein concentrations normalized to 580 μg·mL⁻¹ in their respective buffers. A stock solution of 72.5 mM terbium(III) chloride (TbCl₃) (Sigma-Aldrich, 451304-1G) was prepared in low salt Tev cleavage buffer. To measure the Tb³⁺ luminescence of samples, luminescence emission spectra and intensities were measured on a Synergy™ Neo2 hybrid multi-mode reader (BioTek) in flat bottom, black polystyrene, non-binding surface 96-well half-area microplates (Corning 3686). In technical triplicates, 6.90 μL of 72.5 mM TbCl₃ was mixed with either 43.1 μL of 580 μg·mL⁻¹ protein or 43.1 μL of the corresponding protein sample buffer (either low salt Tev cleavage buffer or PBS) to final concentrations of 10.0 mM TbCl₃ and either 500 μg·mL⁻¹ protein or 0 μg·mL⁻¹ protein in 50.0 μL final volumes per well. Luminescence emission spectra were measured using excitation wavelength λₑₓ=280 nm and emission wavelengths λₑₘ=510-580 nm, and the mean luminescence emission intensity and s.d. of the mean per wavelength reported after smoothing data with Savitzky-Golay filter of order 3 (FIG. 4e). Luminescence intensities were measured using excitation wavelength λₑₓ=280 nm and emission wavelength λₑₘ=544 nm, and background-subtracted data reported by subtracting the mean luminescence intensity of wells with protein sample buffer from the mean luminescence intensity of wells with protein (FIG. 4f).

### Data Availability

Coordinates and structure factors have been deposited in the Research Collaboratory for Structural Bioinformatics Protein Data Bank with the accession codes 7SKN (dIG8-CC, tetragonal), 7SKO (dIG8-CC, orthorhombic) and 7SKP (dIG14). Other data are available from the corresponding authors upon request.

### METHODS REFERENCES

34. W. Kabsch, C. Sander, Dictionary of protein secondary structure: Pattern recognition of hydrogen-bonded and geometrical features. Biopolymers. 22, 2577-2637 (1983).
35. A. Andreeva, E. Kulesha, J. Gough, A. G. Murzin, The SCOP database in 2020: expanded classification of representative family and superfamily domains of known protein structures. Nucleic Acids Research. 48, D376-D382 (2020).
36. S. J. Fleishman et al., RosettaScripts: A Scripting Language Interface to the Rosetta Macromolecular Modeling Suite. PLoS ONE. 6, e20161 (2011).
37. G. Bhardwaj et al., Accurate de novo design of hyperstable constrained peptides. Nature. 538, 329-335 (2016).
38. R. F. Alford et al., The Rosetta All-Atom Energy Function for Macromolecular Modeling and Design. J. Chem. Theory Comput. 13, 3031-3048 (2017).
39. W. Sheffler, D. Baker, RosettaHoles2: A volumetric packing measure for protein structure refinement and validation: RosettaHoles2 for Protein Structure. Protein Science. 19, 1991-1995 (2010).
40. G. C. P. van Zundert et al., The HADDOCK2.2 Web Server: User-Friendly Integrative Modeling of Biomolecular Complexes. Journal of Molecular Biology. 428, 720-725 (2016).
41. A. Courbet et al., “Computational design of nanoscale rotational mechanics in de novo protein assemblies” (preprint, Synthetic Biology, 2021), doi:10.1101/2021.11.11.468255.
42. M. Siedlecka et al., Alpha-helix nucleation by a calcium-binding peptide loop. Proceedings of the National Academy of Sciences. 96, 903-908 (1999).
43. A. S. Ford, B. D. Weitzner, C. D. Bahl, Integration of the Rosetta suite with the python software stack via reproducible packaging and core programming interfaces for distributed simulation. Protein Science. 29, 43-51 (2020).
44. K. H. Le et al., PyRosetta Jupyter Notebooks Teach Biomolecular Structure Prediction and Design. The Biophysicist. 2, 108-122 (2021).
45. M. Rocklin, (Austin, Tex., 2015; conference.scipy.org/proceedings/scipy2015/matthew_rocklin.html), pp. 126-132.
46. T. Brunette et al., Modular repeat protein sculpting using rigid helical junctions. Proc Natl Acad Sci USA. 117, 8870-8875 (2020).
47. F. W. Studier, Protein production by auto-induction in high-density shaking cultures. Protein Expr Purif. 41, 207-234 (2005).
48. I. Anishchenko et al., De novo protein design by deep network hallucination. Nature (2021), doi:10.1038/s41586-021-04184-w.
49. H. Block et al., in Methods in Enzymology (Elsevier, 2009; linkinghub.elsevier.com/retrieve/pii/S0076687909630275), vol. 463, pp. 439-473.
50. J. Juanhuix et al., Developments in optics and performance at BL13-XALOC, the macromolecular crystallography beamline at the Alba Synchrotron. J Synchrotron Rad. 21, 679-689 (2014).
51. D. von Stetten et al., ID30A-3 (MASSIF-3)—a beamline for macromolecular crystallography at the ESRF with a small intense beam. J Synchrotron Rad. 27, 844-851 (2020).
52. W. Kabsch, XDS. Acta Crystallogr D Biol Crystallogr. 66, 125-132 (2010).
53. P. D. Adams et al., PHENIX: a comprehensive Python-based system for macromolecular structure solution. Acta Crystallogr D Biol Crystallogr. 66, 213-221 (2010).
54. M. D. Winn et al., Overview of the CCP4 suite and current developments. Acta Crystallogr D Biol Crystallogr. 67, 235-242 (2011).
55. P. H. Zwart, R. W. Grosse-Kunstleve, P. D. Adams, CCP4 Newsletter on Protein Crystallography Vol. 43 (Winter 2005) (ed F. Remacle) 27-35 (Daresbury Laboratory) (2005).
56. P. R. Evans, An introduction to data reduction: space-group determination, scaling and intensity statistics. Acta Crystallogr D Biol Crystallogr. 67, 282-292 (2011).
57. A. J. McCoy et al., Phaser crystallographic software. J Appl Crystallogr. 40, 658-674 (2007).
58. A. Casafial, B. Lohkamp, P. Emsley, Current developments in Coot for macromolecular model building of Electron Cryo-microscopy and Crystallographic Data. Protein Science. 29, 1055-1064 (2020).
59. D. Liebschner et al., Macromolecular structure determination using X-rays, neutrons and electrons: recent developments in Phenix. Acta Crystallogr D Struct Biol. 75, 861-877 (2019).
60. T. C. Terwilliger et al., Iterative model building, structure refinement and density modification with the PHENIX AutoBuild wizard. Acta Crystallogr D Biol Crystallogr. 64, 61-69(2008).
61. BUSTER version 2.10 (Global Phasing Ltd., Cambridge (UK) (2017).
62. J. C. Klima et al., Bacterial expression and protein purification of mini-fluorescence-activating proteins (2021), doi:10.21203/rs.3.pex-1077/v1.

### Example 2. dIG14-scdim Design

The dIG14 structure was effectively formed by two 6-stranded Ig monomers, suggesting that the C-terminal β-strand was dispensable for proper folding. As the sixth β-strand of one monomer and the first β-strand of the second build an antiparallel interface (and therefore orient their C- and N-termini in close proximity), we reasoned that the two Ig interacting monomers could be fused with a short linker forming a β-hairpin at the interface. Based on the dIG14 crystal structure, we removed the last 9 residues of the sequence, and to find the shortest connection between the two chains we performed Rosetta™ fragment-based insertion of poly-glycine loops (ranging between 2 and 5 amino acids) connecting W68 or G69 of one monomer with G1 or R2 of the other monomer. We found that the gap could be easily bridged with minimal backbone strain with loops equal or larger than 2 connecting G69 with G1. For these single-chain dimers, AlphaFold2™ (AF2) generated highly confident predictions (pLDDT>90) across all residue positions and that matched very closely the design model (Cα-RMSD=0.6 Å).

Encouraged by the confident predictions, we selected the dIG14-scdim designed with a GG linker for experimental characterization, and ordered a synthetic gene encoding for the designed sequence. We express it in Escherichia coli, purified it by affinity and size-exclusion chromatography, and it was found to be well-expressed, soluble and monomeric by size-exclusion chromatography combined with multi-angle light-scattering (SEC-MALS). Moreover, it was found to have far-UV circular dichroism spectra characteristic of all-β proteins and turned out to be hyperstable by circular dichroism—the protein remains folded in 6 M GdnCl. We succeeded in solving a crystal structure of dIG14-scdim at 2.8 Å resolution, and was found in excellent agreement with the computational model across the 12 β-strands (Cα-RMSD=0.8 Å). This structure can be regarded as a flattened β-barrel. At the bottom, the designed linker is surrounded by a tightly packed area stabilized by aromatic stacking, and at the top it was found a cavity binding a glycerol molecule (as crystallization component) that is surrounded by the two β-arch helices—the structure of this area thus could be also diversified for designing small-molecule binding sites. The complex β-sheet arrangement of the structure constitutes a novel all-β domain topology, given that the closest structural analogues found in the PDB or the AlphaFold™ Structure Database had low TM-scores (<=0.65), and those were β-sandwiches formed by a different number of β-strands and strand pairing organization.

We claim:

1. A polypeptide comprising the formula X1-X2-X3-X4-X5-X6-X7-X8-X9-X10-X11-X12-X13-X14-X15, wherein:
X1 is optional, and when present comprises 1 2, or 3 residues with loop secondary structure;
X2 comprises 5, 6, 7, or 8 residues with β-strand secondary structure;
X3 comprises 2, 3 or 4 residues with loop secondary structure, forming a β-hairpin tertiary structure motif;
X4 comprises 6, 7, or 8 residues with β-strand secondary structure;
X5 comprises 3, 4, 5 or 6 residues with loop secondary structure, forming a β-arch tertiary structure motif (i.e., a connection between X4 and X6);
X6 comprises 6 or 7 residues with β-strand secondary structure;
X7 comprises 2, 3, or 4 residues with loop secondary structure, forming a β-hairpin tertiary structure motif;
X8 comprises 6 or 7 residues with β-strand secondary structure;
X9 comprises 3, 4 or 5 residues with loop secondary structure, forming a β-arch tertiary structure motif (i.e., a connection between X8 and X10);
X10 comprises 4, 5, 6, 7 or 8 residues with β-strand secondary structure;
X11 comprises one of the following, forming a β-arch tertiary structure motif:
3, 4, 5, 6, 7, or 8 residues with loop secondary structure; or
2, 3, or 4 residues with loop secondary structure, followed by 3, 4, 5, or 6 residues with α-helical secondary structure, followed by 1, 2, or 3 residues with loop secondary structure;
X12 comprises 6, 7, or 8 residues with β-strand secondary structure;
X13 comprises 2, 3, or 4 residues with loop secondary structure, forming a β-hairpin tertiary structure motif;
X14 comprises 5, 6, 7, or 8 residues with β-strand secondary structure; and
X15 is optional, and when present comprises 1, 2 or 3 residues with loop secondary structure.

2. The polypeptide of claim 1, wherein: neither X1 nor X15 are present; one of X1 or X15 is present (for example, X1 is present; or X15 is present); or X1 and X15 are both present.

3. The polypeptide of claim 1, wherein 1, 2, or all 3 β-arch motifs (X5, X9, and X11) have atoms involved in hydrogen bonds between (i) two backbone atoms, (ii) one backbone and a sidechain atom, or (iii) two-sidechain atoms.

4. The polypeptide of claim 1, wherein 1, 2, 3, 4, 5, 6, 7, or all 8 of the following are true:
(a) X2 forms an antiparallel β-strand pairing with X4;
(b) X4 forms an antiparallel β-strand pairing with X10;
(c) X2, X4, and X10 form a first layer of β-sheets, with X2 and X10 as edge β-strands;
(d) X6 forms an antiparallel β-strand pairing with X8;
(e) X6 forms an antiparallel β-strand pairing with X12;
(f) X12 forms an antiparallel β-strand pairing with X14;
(g) X6, X8, X12 and X14 form a second layer of β-sheets, with X8 and X14 as edge β-strands; and/or
(h) the first layer of β-sheets and the second layer of β-sheets form a β-sandwich tertiary structure motif.

5. The polypeptide of claim 1, wherein X4, X6, and X12 comprise alternating hydrophobic and hydrophilic residues, and optionally wherein 1, 2, 3, or all 4 of X2, X8, X10, and X14 comprise alternating hydrophobic and hydrophilic residues.

6. The polypeptide of claim 1, where X4, X6, and X12 independently comprise the amino acid sequence selected from the group consisting of SEQ ID NO:1-87.

7. The polypeptide of claim 1, wherein 1, 2, 3, or all 4 of X2, X8, X10, and X14 comprise alternating hydrophobic and hydrophilic residues, independently comprising the amino acid sequence selected from the following group consisting of SEQ ID NO:88-123.

8. The polypeptide of claim 1, wherein X2, X8, X10, and X14 comprise at least one polar amino acid residue selected from Arg, Lys, Glu, Gln, and His.

9. The polypeptide of claim 8, wherein X2, X8, X10, and X14 independently comprise the amino acid sequence selected from the group consisting of SEQ ID NO:88-203.

10. The polypeptide of claim 1, wherein X2, X4, X6, X8, X10, X12, and X14 independently comprise an amino acid sequence selected from the group consisting of SEQ ID NO:1-203.

11. The polypeptide of claim 1, wherein X5, X9, and X11 comprise (i) at least one polar amino acid selected from Asn, Ser, Thr, Glu, and Gln in the domain or in the residue immediately preceding or following the domain, where the polar residue is involved in at least one hydrogen bond between (i) two backbone atoms, (ii) one backbone and a sidechain atom, or (iii) two-sidechain atoms, and (iv) a glycine or proline residue.

12. The polypeptide of claim 1, wherein the X3, X7, and X13 domains each comprise at least one glycine residue.

13. The polypeptide of claim 1, wherein at least two non-contiguous β-strands include a cysteine residue, wherein the at least two non-contiguous β-strand cysteine residues are capable of forming a disulfide bond.

14. The polypeptide of claim 1, comprising an amino acid sequence at least 50% identical, not including any functional domain insertions, to the amino acid sequence selected from group consisting of SEQ ID NO: 204-235 and 291-301.

15. A polypeptide comprising an amino acid sequence at least 50% identical, not including any functional domain insertions, to the amino acid sequence selected from the following group consisting of SEQ ID NO: 204-235 and 291-301.

16. The polypeptide of claim 1, further comprising one or more functional domains inserted into the polypeptide.

17. A multimer, comprising 2, or more copies of the polypeptide of claim 1.

18. A nucleic acid encoding the polypeptide of claim 1.

19. An expression vector, comprising the nucleic acid of claim 18 operatively linked to a suitable control sequence.

20. A host cell comprising the expression vector of claim 19.


SEQUENCE LISTING (303 sequences)

SEQ ID NO: 1 - protein, 7 aa, synthetic construct
  REGION 1..7: note = Synthetic peptide
AEIEVRW

SEQ ID NO: 2 - protein, 7 aa, synthetic construct
  REGION 1..7: note = Synthetic peptide
AEIHLEF

SEQ ID NO: 3 - protein, 7 aa, synthetic construct
  REGION 1..7: note = Synthetic peptide
AEVEVEC

SEQ ID NO: 4 - protein, 7 aa, synthetic construct
  REGION 1..7: note = Synthetic peptide
AEVEVEV

SEQ ID NO: 5 - protein, 7 aa, synthetic construct
  REGION 1..7: note = Synthetic peptide
AEVRFHY

SEQ ID NO: 6 - protein, 7 aa, synthetic construct
  REGION 1..7: note = Synthetic peptide
AEVRIEK

SEQ ID NO: 7 - protein, 7 aa, synthetic construct
  REGION 1..7: note = Synthetic peptide
AEVRLEI

SEQ ID NO: 8 - protein, 7 aa, synthetic construct
  REGION 1..7: note = Synthetic peptide
ARIEIKV

SEQ ID NO: 9 - protein, 7 aa, synthetic construct
  REGION 1..7: note = Synthetic peptide
ARVEFEY

SEQ ID NO: 10 - protein, 7 aa, synthetic construct
  REGION 1..7: note = Synthetic peptide
ARVEIEV

SEQ ID NO: 11 - protein, 7 aa, synthetic construct
  REGION 1..7: note = Synthetic peptide
ARVEMEV

SEQ ID NO: 12 - protein, 7 aa, synthetic construct
  REGION 1..7: note = Synthetic peptide
ARVEVEE

SEQ ID NO: 13 - protein, 7 aa, synthetic construct
  REGION 1..7: note = Synthetic peptide
ATLEVEK

SEQ ID NO: 14 - protein, 7 aa, synthetic construct
  REGION 1..7: note = Synthetic peptide
ATVHVEK

SEQ ID NO: 15 - protein, 7 aa, synthetic construct
  REGION 1..7: note = Synthetic peptide
ATVRIEY

SEQ ID NO: 16 - protein, 7 aa, synthetic construct
  REGION 1..7: note = Synthetic peptide
CRVRVTA

SEQ ID NO: 17 - protein, 6 aa, synthetic construct
  REGION 1..6: note = Synthetic peptide
EFHVEA

SEQ ID NO: 18 - protein, 6 aa, synthetic construct
  REGION 1..6: note = Synthetic peptide
EIEVEV

SEQ ID NO: 19 - protein, 8 aa, synthetic construct
  REGION 1..8: note = Synthetic peptide
EVEVEVKA

SEQ ID NO: 20 - protein, 8 aa, synthetic construct
  REGION 1..8: note = Synthetic peptide
EVEVTVEY

SEQ ID NO: 21 - protein, 8 aa, synthetic construct
  REGION 1..8: note = Synthetic peptide
EVRVEVEM

SEQ ID NO: 22 - protein, 8 aa, synthetic construct
  REGION 1..8: note = Synthetic peptide
EYEVEVEN

SEQ ID NO: 23 - protein, 7 aa, synthetic construct
  REGION 1..7: note = Synthetic peptide
GKVRIEF

SEQ ID NO: 24 - protein, 7 aa, synthetic construct
  REGION 1..7: note = Synthetic peptide
GRLELEY

SEQ ID NO: 25 - protein, 7 aa, synthetic construct
  REGION 1..7: note = Synthetic peptide
GRVEFEV

SEQ ID NO: 26 - protein, 7 aa, synthetic construct
  REGION 1..7: note = Synthetic peptide
GRVEIKA

SEQ ID NO: 27 - protein, 7 aa, synthetic construct
  REGION 1..7: note = Synthetic peptide
GRVEVQV

SEQ ID NO: 28 - protein, 7 aa, synthetic construct
  REGION 1..7: note = Synthetic peptide
GRVEVRF

SEQ ID NO: 29 - protein, 7 aa, synthetic construct
  REGION 1..7: note = Synthetic peptide
GTIEIEV

SEQ ID NO: 30 - protein, 7 aa, synthetic construct
  REGION 1..7: note = Synthetic peptide
GTVEIEV

SEQ ID NO: 31 - protein, 7 aa, synthetic construct
  REGION 1..7: note = Synthetic peptide
GTVEVEV

SEQ ID NO: 32 - protein, 7 aa, synthetic construct
  REGION 1..7: note = Synthetic peptide
IRFEMTM

SEQ ID NO: 33 - protein, 7 aa, synthetic construct
  REGION 1..7: note = Synthetic peptide
IRFEMVM

SEQ ID NO: 34 - protein, 7 aa, synthetic construct
  REGION 1..7: note = Synthetic peptide
IRFEVEV

SEQ ID NO: 35 - protein, 7 aa, synthetic construct
  REGION 1..7: note = Synthetic peptide
IRVEVEV

SEQ ID NO: 36 - protein, 6 aa, synthetic construct
  REGION 1..6: note = Synthetic peptide
KFEFRN

SEQ ID NO: 37 - protein, 6 aa, synthetic construct
  REGION 1..6: note = Synthetic peptide
KIEVEN

SEQ ID NO: 38 - protein, 8 aa, synthetic construct
  REGION 1..8: note = Synthetic peptide
KIEVRVTN

SEQ ID NO: 39 - protein, 7 aa, synthetic construct
  REGION 1..7: note = Synthetic peptide
KMRVRLR

SEQ ID NO: 40 - protein, 8 aa, synthetic construct
  REGION 1..8: note = Synthetic peptide
KMRVRLRN

SEQ ID NO: 41 - protein, 8 aa, synthetic construct
  REGION 1..8: note = Synthetic peptide
KVEIRVRS

SEQ ID NO: 42 - protein, 8 aa, synthetic construct
  REGION 1..8: note = Synthetic peptide
KVEVHVEA

SEQ ID NO: 43 - protein, 6 aa, synthetic construct
  REGION 1..6: note = Synthetic peptide
KVEVRN

SEQ ID NO: 44 - protein, 6 aa, synthetic construct
  REGION 1..6: note = Synthetic peptide
KVHIRT

SEQ ID NO: 45 - protein, 8 aa, synthetic construct
  REGION 1..8: note = Synthetic peptide
KYELRIRN

SEQ ID NO: 46 - protein, 7 aa, synthetic construct
  REGION 1..7: note = Synthetic peptide
MRIEVRA

SEQ ID NO: 47 - protein, 7 aa, synthetic construct
  REGION 1..7: note = Synthetic peptide
MRVEFTW

SEQ ID NO: 48 - protein, 6 aa, synthetic construct
  REGION 1..6: note = Synthetic peptide
QFTIEW

SEQ ID NO: 49 - protein, 8 aa, synthetic construct
  REGION 1..8: note = Synthetic peptide
QWEVRIRN

SEQ ID NO: 50 - protein, 8 aa, synthetic construct
  REGION 1..8: note = Synthetic peptide
RIEIQIHN

SEQ ID NO: 51 - protein, 8 aa, synthetic construct
  REGION 1..8: note = Synthetic peptide
RIRVEVQE

SEQ ID NO: 52 - protein, 6 aa, synthetic construct
  REGION 1..6: note = Synthetic peptide
RIRVRN

SEQ ID NO: 53 - protein, 8 aa, synthetic construct
  REGION 1..8: note = Synthetic peptide
RLEVEIRN

SEQ ID NO: 54 - protein, 8 aa, synthetic construct
  REGION 1..8: note = Synthetic peptide
RLRIHVEV

SEQ ID NO: 55 - protein, 7 aa, synthetic construct
  REGION 1..7: note = Synthetic peptide
RVEIEIV

SEQ ID NO: 56 - protein, 6 aa, synthetic construct
  REGION 1..6: note = Synthetic peptide
RVEIEN

SEQ ID NO: 57 - protein, 6 aa, synthetic construct
  REGION 1..6: note = Synthetic peptide
RVEIEY

SEQ ID NO: 58 - protein, 8 aa, synthetic construct
  REGION 1..8: note = Synthetic peptide
RVEVRVEN

SEQ ID NO: 59 - protein, 8 aa, synthetic construct
  REGION 1..8: note = Synthetic peptide
RVEVRVQS

SEQ ID NO: 60 - protein, 6 aa, synthetic construct
  REGION 1..6: note = Synthetic peptide
RVRLEY

SEQ ID NO: 61 - protein, 6 aa, synthetic construct
  REGION 1..6: note = Synthetic peptide
RVRVRN

SEQ ID NO: 62 - protein, 6 aa, synthetic construct
  REGION 1..6: note = Synthetic peptide
RYEFRN

SEQ ID NO: 63 - protein, 8 aa, synthetic construct
  REGION 1..8: note = Synthetic peptide
RYRLEIRT

SEQ ID NO: 64 - protein, 8 aa, synthetic construct
  REGION 1..8: note = Synthetic peptide
TFEVEIRN

SEQ ID NO: 65 - protein, 8 aa, synthetic construct
  REGION 1..8: note = Synthetic peptide
TIEFRFEN

SEQ ID NO: 66 - protein, 6 aa, synthetic construct
  REGION 1..6: note = Synthetic peptide
TIEIRS

SEQ ID NO: 67 - protein, 8 aa, synthetic construct
  REGION 1..8: note = Synthetic peptide
TIHVEVEN

SEQ ID NO: 68 - protein, 6 aa, synthetic construct
  REGION 1..6: note = Synthetic peptide
TIQLHQ

SEQ ID NO: 69 - protein, 6 aa, synthetic construct
  REGION 1..6: note = Synthetic peptide
TIRVEN

SEQ ID NO: 70 - protein, 7 aa, synthetic construct
  REGION 1..7: note = Synthetic peptide
TIRVEVR

SEQ ID NO: 71 - protein, 8 aa, synthetic construct
  REGION 1..8: note = Synthetic peptide
TIRVTVEN

SEQ ID NO: 72 - protein, 6 aa, synthetic construct
  REGION 1..6: note = Synthetic peptide
TVEVTV

SEQ ID NO: 73 - protein, 7 aa, synthetic construct
  REGION 1..7: note = Synthetic peptide
VEVHIKV

SEQ ID NO: 74 - protein, 7 aa, synthetic construct
  REGION 1..7: note = Synthetic peptide
VEVRMEK

SEQ ID NO: 75 - protein, 7 aa, synthetic construct
  REGION 1..7: note = Synthetic peptide
VEVRVRA

SEQ ID NO: 76 - protein, 7 aa, synthetic construct
  REGION 1..7: note = Synthetic peptide
VEVRVRN

SEQ ID NO: 77 - protein, 7 aa, synthetic construct
  REGION 1..7: note = Synthetic peptide
VHVEVEI

SEQ ID NO: 78 - protein, 7 aa, synthetic construct
  REGION 1..7: note = Synthetic peptide
VRFRVVG

SEQ ID NO: 79 - protein, 7 aa, synthetic construct
  REGION 1..7: note = Synthetic peptide
VRFTVEM

SEQ ID NO: 80 - protein, 6 aa, synthetic construct
  REGION 1..6: note = Synthetic peptide
VRIRVT

SEQ ID NO: 81 - protein, 7 aa, synthetic construct
  REGION 1..7: note = Synthetic peptide
VRITVEV

SEQ ID NO: 82 - protein, 7 aa, synthetic construct
  REGION 1..7: note = Synthetic peptide
VRVEVEV

SEQ ID NO: 83 - protein, 7 aa, synthetic construct
  REGION 1..7: note = Synthetic peptide
VRVEVKA

SEQ ID NO: 84 - protein, 7 aa, synthetic construct
  REGION 1..7: note = Synthetic peptide
VRVEVVY

SEQ ID NO: 85 - protein, 7 aa, synthetic construct
  REGION 1..7: note = Synthetic peptide
VRVRVTA

SEQ ID NO: 86 - protein, 7 aa, synthetic construct
  REGION 1..7: note = Synthetic peptide
VTLEVEF

SEQ ID NO: 87 - protein, 7 aa, synthetic construct
  REGION 1..7: note = Synthetic peptide
YRVEVEN

SEQ ID NO: 88 - protein, 7 aa, synthetic construct
  REGION 1..7: note = Synthetic peptide
AKYELKG

SEQ ID NO: 89 - protein, 7 aa, synthetic construct
  REGION 1..7: note = Synthetic peptide
AKYEVEL

SEQ ID NO: 90 - protein, 4 aa, synthetic construct
  REGION 1..4: note = Synthetic peptide
ELEI

SEQ ID NO: 91 - protein, 7 aa, synthetic construct
  REGION 1..7: note = Synthetic peptide
ELEIRMD

SEQ ID NO: 92 - protein, 6 aa, synthetic construct
  REGION 1..6: note = Synthetic peptide
ELELHF

SEQ ID NO: 93 - protein, 5 aa, synthetic construct
  REGION 1..5: note = Synthetic peptide
ELEVE

SEQ ID NO: 94 - protein, 6 aa, synthetic construct
  REGION 1..6: note = Synthetic peptide
ELRIEF

SEQ ID NO: 95 - protein, 7 aa, synthetic construct
  REGION 1..7: note = Synthetic peptide
EVEVTFS

SEQ ID NO: 96 - protein, 8 aa, synthetic construct
  REGION 1..8: note = Synthetic peptide
FEVRVQWS

SEQ ID NO: 97 - protein, 6 aa, synthetic construct
  REGION 1..6: note = Synthetic peptide
IEVRVD

SEQ ID NO: 98 - protein, 6 aa, synthetic construct
  REGION 1..6: note = Synthetic peptide
IEVRVK

SEQ ID NO: 99 - protein, 7 aa, synthetic construct
  REGION 1..7: note = Synthetic peptide
KVQLELH

SEQ ID NO: 100 - protein, 7 aa, synthetic construct
  REGION 1..7: note = Synthetic peptide
KYSYEYT

SEQ ID NO: 101 - protein, 4 aa, synthetic construct
  REGION 1..4: note = Synthetic peptide
LEIR

SEQ ID NO: 102 - protein, 6 aa, synthetic construct
  REGION 1..6: note = Synthetic peptide
LEVRME

SEQ ID NO: 103 - protein, 6 aa, synthetic construct
  REGION 1..6: note = Synthetic peptide
MEVRVS

SEQ ID NO: 104 - protein, 4 aa, synthetic construct
  REGION 1..4: note = Synthetic peptide
MEYR

SEQ ID NO: 105 - protein, 7 aa, synthetic construct
  REGION 1..7: note = Synthetic peptide
QMRVEIS

SEQ ID NO: 106 - protein, 7 aa, synthetic construct
  REGION 1..7: note = Synthetic peptide
RIEVEVT

SEQ ID NO: 107 - protein, 7 aa, synthetic construct
  REGION 1..7: note = Synthetic peptide
RLELHFQ

SEQ ID NO: 108 - protein, 7 aa, synthetic construct
  REGION 1..7: note = Synthetic peptide
RLTVEFT

SEQ ID NO: 109 - protein, 5 aa, synthetic construct
  REGION 1..5: note = Synthetic peptide
RVEVE

SEQ ID NO: 110 - protein, 7 aa, synthetic construct
  REGION 1..7: note = Synthetic peptide
RVRFEFR

SEQ ID NO: 111 - protein, 5 aa, synthetic construct
  REGION 1..5: note = Synthetic peptide
SVEVR

SEQ ID NO: 112 - protein, 5 aa, synthetic construct
  REGION 1..5: note = Synthetic peptide
TIEVQ

SEQ ID NO: 113 - protein, 6 aa, synthetic construct
  REGION 1..6: note = Synthetic peptide
TLRLRG

SEQ ID NO: 114 - protein, 6 aa, synthetic construct
  REGION 1..6: note = Synthetic peptide
TVRVEF

SEQ ID NO: 115 - protein, 6 aa, synthetic construct
  REGION 1..6: note = Synthetic peptide
VEIRYK

SEQ ID NO: 116 - protein, 6 aa, synthetic construct
  REGION 1..6: note = Synthetic peptide
VEVRIS

SEQ ID NO: 117 - protein, 6 aa, synthetic construct
  REGION 1..6: note = Synthetic peptide
VEVRLE

SEQ ID NO: 118 - protein, 8 aa, synthetic construct
  REGION 1..8: note = Synthetic peptide
VEVRVEFE

SEQ ID NO: 119 - protein, 7 aa, synthetic construct
  REGION 1..7: note = Synthetic peptide
VEVRVTY

SEQ ID NO: 120 - protein, 7 aa, synthetic construct
  REGION 1..7: note = Synthetic peptide
VRLRFTV

SEQ ID NO: 121 - protein, 5 aa, synthetic construct
  REGION 1..5: note = Synthetic peptide
VRVEV

SEQ ID NO: 122 - protein, 8 aa, synthetic construct
  REGION 1..8: note = Synthetic peptide
VRWTWRIS

SEQ ID NO: 123 - protein, 8 aa, synthetic construct
  REGION 1..8: note = Synthetic peptide
WEVRVRWK

SEQ ID NO: 124 - protein, 7 aa, synthetic construct
  REGION 1..7: note = Synthetic peptide
EEVEIEV

SEQ ID NO: 125 - protein, 7 aa, synthetic construct
  REGION 1..7: note = Synthetic peptide
EKRTVTV

SEQ ID NO: 126 - protein, 8 aa, synthetic construct
  REGION 1..8: note = Synthetic peptide
EKRTYEFQ

SEQ ID NO: 127 - protein, 8 aa, synthetic construct
  REGION 1..8: note = Synthetic peptide
EKVRTRYR

SEQ ID NO: 128 - protein, 7 aa, synthetic construct
  REGION 1..7: note = Synthetic peptide
EKYEVRI

SEQ ID NO: 129 - protein, 7 aa, synthetic construct
  REGION 1..7: note = Synthetic peptide
EKYRFQL

SEQ ID NO: 130 - protein, 6 aa, synthetic construct
  REGION 1..6: note = Synthetic peptide
EKYTYT

SEQ ID NO: 131 - protein, 6 aa, synthetic construct
  REGION 1..6: note = Synthetic peptide
ERIEVQ

SEQ ID NO: 132 - protein, 7 aa, synthetic construct
  REGION 1..7: note = Synthetic peptide
ERIQFEA

SEQ ID NO: 133 - protein, 6 aa, synthetic construct
  REGION 1..6: note = Synthetic peptide
ETLEVE

SEQ ID NO: 134 - protein, 7 aa, synthetic construct
  REGION 1..7: note = Synthetic peptide
ETREYTV

SEQ ID NO: 135 - protein, 7 aa, synthetic construct
  REGION 1..7: note = Synthetic peptide
ETVEVEV

SEQ ID NO: 136 - protein, 7 aa, synthetic construct
  REGION 1..7: note = Synthetic peptide
EVRVQEK

SEQ ID NO: 137 - protein, 8 aa, synthetic construct
  REGION 1..8: note = Synthetic peptide
FRVEVREK

SEQ ID NO: 138 - protein, 8 aa, synthetic construct
  REGION 1..8: note = Synthetic peptide
IHVELRKE

SEQ ID NO: 139 - protein, 8 aa, synthetic construct
  REGION 1..8: note = Synthetic peptide
IRIEVRSS

SEQ ID NO: 140 - protein, 7 aa, synthetic construct
  REGION 1..7: note = Synthetic peptide
IRVEIEK

SEQ ID NO: 141 - protein, 7 aa, synthetic construct
  REGION 1..7: note = Synthetic peptide
KKFTYTV

SEQ ID NO: 142 - protein, 7 aa, synthetic construct
  REGION 1..7: note = Synthetic peptide
KKLEYQY

SEQ ID NO: 143 - protein, 7 aa, synthetic construct
  REGION 1..7: note = Synthetic peptide
KKYEYTV

SEQ ID NO: 144 - protein, 7 aa, synthetic construct
  REGION 1..7: note = Synthetic peptide
KREERTF

SEQ ID NO: 145 - protein, 7 aa, synthetic construct
  REGION 1..7: note = Synthetic peptide
KREEYHM

SEQ ID NO: 146 - protein, 8 aa, synthetic construct
  REGION 1..8: note = Synthetic peptide
KRERWRFR

SEQ ID NO: 147 - protein, 7 aa, synthetic construct
  REGION 1..7: note = Synthetic peptide
KRERYTL

SEQ ID NO: 148 - protein, 7 aa, synthetic construct
  REGION 1..7: note = Synthetic peptide
KRETYTV

SEQ ID NO: 149 - protein, 7 aa, synthetic construct
  REGION 1..7: note = Synthetic peptide
KRHEVHL

SEQ ID NO: 150 - protein, 7 aa, synthetic construct
  REGION 1..7: note = Synthetic peptide
KRYEYTA

SEQ ID NO: 151 - protein, 6 aa, synthetic construct
  REGION 1..6: note = Synthetic peptide
KTEETQ

SEQ ID NO: 152 - protein, 6 aa, synthetic construct
  REGION 1..6: note = Synthetic peptide
LEVRRK

SEQ ID NO: 153 - protein, 8 aa, synthetic construct
  REGION 1..8: note = Synthetic peptide
LEVRVRKS

SEQ ID NO: 154 - protein, 7 aa, synthetic construct
  REGION 1..7: note = Synthetic peptide
QREEYRM

SEQ ID NO: 155 - protein, 7 aa, synthetic construct
  REGION 1..7: note = Synthetic peptide
QREEYTL

SEQ ID NO: 156 - protein, 7 aa, synthetic construct
  REGION 1..7: note = Synthetic peptide
QRERYHV

SEQ ID NO: 157 - protein, 7 aa, synthetic construct
  REGION 1..7: note = Synthetic peptide
QRETYTA

SEQ ID NO: 158 - protein, 7 aa, synthetic construct
  REGION 1..7: note = Synthetic peptide
QRETYTV

SEQ ID NO: 159 - protein, 8 aa, synthetic construct
  REGION 1..8: note = Synthetic peptide
QRFTFEFT

SEQ ID NO: 160 - protein, 7 aa, synthetic construct
  REGION 1..7: note = Synthetic peptide
QRFTYRY

SEQ ID NO: 161 - protein, 6 aa, synthetic construct
  REGION 1..6: note = Synthetic peptide
QRQHFQ

SEQ ID NO: 162 - protein, 6 aa, synthetic construct
  REGION 1..6: note = Synthetic peptide
QRQRYQ

SEQ ID NO: 163 - protein, 7 aa, synthetic construct
  REGION 1..7: note = Synthetic peptide
QRREYTG

SEQ ID NO: 164 - protein, 7 aa, synthetic construct
  REGION 1..7: note = Synthetic peptide
QRREYTV

SEQ ID NO: 165 - protein, 7 aa, synthetic construct
  REGION 1..7: note = Synthetic peptide
QRYEFTA

SEQ ID NO: 166 - protein, 7 aa, synthetic construct
  REGION 1..7: note = Synthetic peptide
QRYEYHM

SEQ ID NO: 167 - protein, 6 aa, synthetic construct
  REGION 1..6: note = Synthetic peptide
QRYRFE

SEQ ID NO: 168 - protein, 8 aa, synthetic construct
  REGION 1..8: note = Synthetic peptide
QRYTFEVR

SEQ ID NO: 169 - protein, 8 aa, synthetic construct
  REGION 1..8: note = Synthetic peptide
QRYTYEVR

SEQ ID NO: 170 - protein, 7 aa, synthetic construct
  REGION 1..7: note = Synthetic peptide
QRYTYTL

SEQ ID NO: 171 - protein, 5 aa, synthetic construct
  REGION 1..5: note = Synthetic peptide
QTVEY

SEQ ID NO: 172 - protein, 7 aa, synthetic construct
  REGION 1..7: note = Synthetic peptide
REYRYKL

SEQ ID NO: 173 - protein, 8 aa, synthetic construct
  REGION 1..8: note = Synthetic peptide
RKIETELT

SEQ ID NO: 174 - protein, 7 aa, synthetic construct
  REGION 1..7: note = Synthetic peptide
RLRYRTK

SEQ ID NO: 175 - protein, 7 aa, synthetic construct
  REGION 1..7: note = Synthetic peptide
RREEYTA

SEQ ID NO: 176 - protein, 7 aa, synthetic construct
  REGION 1..7: note = Synthetic peptide
RREEYTL

SEQ ID NO: 177 - protein, 7 aa, synthetic construct
  REGION 1..7: note = Synthetic peptide
RRERYEA

SEQ ID NO: 178 - protein, 7 aa, synthetic construct
  REGION 1..7: note = Synthetic peptide
RRERYTA

SEQ ID NO: 179 - protein, 7 aa, synthetic construct
  REGION 1..7: note = Synthetic peptide
RRETYTV

SEQ ID NO: 180 - protein, 7 aa, synthetic construct
  REGION 1..7: note = Synthetic peptide
RRFTYTG

SEQ ID NO: 181 - protein, 6 aa, synthetic construct
  REGION 1..6: note = Synthetic peptide
RRYEVR

SEQ ID NO: 182 - protein, 6 aa, synthetic construct
  REGION 1..6: note = Synthetic peptide
SELRVT

SEQ ID NO: 183 - protein, 8 aa, synthetic construct
  REGION 1..8: note = Synthetic peptide
SEVHVRFE

SEQ ID NO: 184 - protein, 7 aa, synthetic construct
  REGION 1..7: note = Synthetic peptide
STKWRFE

SEQ ID NO: 185 - protein, 7 aa, synthetic construct
  REGION 1..7: note = Synthetic peptide
TEMRVEI

SEQ ID NO: 186 - protein, 7 aa, synthetic construct
  REGION 1..7: note = Synthetic peptide
TEVRVEQ

SEQ ID NO: 187 - protein, 6 aa, synthetic construct
  REGION 1..6: note = Synthetic peptide
TLETTA

SEQ ID NO: 188 - protein, 8 aa, synthetic construct
  REGION 1..8: note = Synthetic peptide
TQYRYEFE

SEQ ID NO: 189 - protein, 6 aa, synthetic construct
  REGION 1..6: note = Synthetic peptide
TRIEFE

SEQ ID NO: 190 - protein, 6 aa, synthetic construct
  REGION 1..6: note = Synthetic peptide
TRYEYE

SEQ ID NO: 191 - protein, 7 aa, synthetic construct
  REGION 1..7: note = Synthetic peptide
TTETYTV

SEQ ID NO: 192 - protein, 8 aa, synthetic construct
  REGION 1..8: note = Synthetic peptide
TTITVEGT

SEQ ID NO: 193 - protein, 7 aa, synthetic construct
  REGION 1..7: note = Synthetic peptide
TTYEYTV

SEQ ID NO: 194 - protein, 7 aa, synthetic construct
  REGION 1..7: note = Synthetic peptide
TTYQRTI

SEQ ID NO: 195 - protein, 7 aa, synthetic construct
  REGION 1..7: note = Synthetic peptide
TTYRYEV

SEQ ID NO: 196 - protein, 7 aa, synthetic construct
  REGION 1..7: note = Synthetic peptide
TTYRYRL

SEQ ID NO: 197 - protein, 8 aa, synthetic construct
  REGION 1..8: note = Synthetic peptide
VEFRLREE

SEQ ID NO: 198 - protein, 8 aa, synthetic construct
  REGION 1..8: note = Synthetic peptide
VEIEVRTK

SEQ ID NO: 199 - protein, 8 aa, synthetic construct
  REGION 1..8: note = Synthetic peptide
VEVRIREE

SEQ ID NO: 200 - protein, 8 aa, synthetic construct
  REGION 1..8: note = Synthetic peptide
VEVRIRKN

SEQ ID NO: 201 - protein, 8 aa, synthetic construct
  REGION 1..8: note = Synthetic peptide
VEVRITEK

SEQ ID NO: 202 - protein, 6 aa, synthetic construct
  REGION 1..6: note = Synthetic peptide
VEVRQS

SEQ ID NO: 203 - protein, 7 aa, synthetic construct
  REGION 1..7: note = Synthetic peptide
VRTSYTM

SEQ ID NO: 204 - protein, 72 aa, synthetic construct
  REGION 1..72: note = Synthetic peptide
TVEVRIRKNGNEYEVEVENRSDRPAEVRFHYDGTTETYTVPPGTRLRYRTKLTKPMRIEVRAGNTTYEYTVS

SEQ ID NO: 205 - protein, 72 aa, synthetic construct
  REGION 1..72: note = Synthetic peptide
EIHVELRKEGDRVEVRVENRSSQPGTVEIEVDGQRYEFTANPGERIQFEARGKTPVRVEVVYGNTTYRYEVR

SEQ ID NO: 206 - protein, 70 aa, synthetic construct
  REGION 1..70: note = Synthetic peptide
RVRVEVKNNKIEVENNSDQPAEIHLEFGGRRFTYTGNKGERIEVQISPEEAKNARIEIKVGDKKLEYQYH

SEQ ID NO: 207 - protein, 70 aa, synthetic construct
  REGION 1..70: note = Synthetic peptide
RVEVRISGNTIRVENRSDRPARVEFEYGGRREEYTAPPGSELRVTISPEELKNARVEIEYGGQRYRFEVT

SEQ ID NO: 208 - protein, 76 aa, synthetic construct
  REGION 1..76: note = Synthetic peptide
KIRIEVRSSGNTIHVEVENNSDRPVRIRVTAPGTTLETTANPGERVRFEFRGVPPGGEVEVEVKAGDEKVRTRYRS

SEQ ID NO: 209 - protein, 72 aa, synthetic construct
  REGION 1..72: note = Synthetic peptide
TVEVRITEKNGQWEVRIRNRSSQPARVEVEEGGRREEYTLNPGDELELHFTSPKPVRITVEVGGQRYTYTLR

SEQ ID NO: 210 - protein, 70 aa, synthetic construct
  REGION 1..70: note = Synthetic peptide
RMEVRVSNGRVEIENKSSQPGRVEVRFNGKRYEYTANPGERVEVEVSPEELKNLRVRLEYDGKTEETQYS

SEQ ID NO: 211 - protein, 70 aa, synthetic construct
  REGION 1..70: note = Synthetic peptide
RIEVRVDNGRVRVRNGTDRPVRVRVTAGGETREYTVNPGTELEVELSPEQQNNAEVEVEVGNEKYRFQLG

SEQ ID NO: 212 - protein, 72 aa, synthetic construct
  REGION 1..72: note = Synthetic peptide
SIRVEIEKRGDSYRVEVENRSDQPAEIEVRWNGRRERYEANKGETVEVEVRAPSPVEVRVRAGNTEVRVEQR

SEQ ID NO: 213 - protein, 67 aa, synthetic construct
  REGION 1..67: note = Synthetic peptide
RVEVRISGNTIEIRSEGPGRLELEYNGQREEYTLNPGTRIEFEGRPGEEVRVEVEMNGQRYTFEVRF

SEQ ID NO: 214 - protein, 69 aa, synthetic construct
  REGION 1..69: note = Synthetic peptide
RLEVRMEGKKVEVRNNSDRPMRVEFTWNGQRERYHVNPGETLEVEVQPGARVEVRVQSGDTQYRYEFEL

SEQ ID NO: 215 - protein, 72 aa, synthetic construct
  REGION 1..72: note = Synthetic peptide
SLEVRVRKSGNTFEVEIRNKSDRPAEVRLEIGGRRETYTVPPGSTLRLRGPGKRPGRVEIKAGDAKYEVELR

SEQ ID NO: 216 - protein, 66 aa, synthetic construct
  REGION 1..66: note = Synthetic peptide
YVEIRYKGEKVHIRTNGPVTLEVEFEGKRERYTLNPGEELEIRIRARRIRVEVQEGDRKIETELTF

SEQ ID NO: 217 - protein, 77 aa, synthetic construct
  REGION 1..77: note = Synthetic peptide
RVEVRVEFEGDKMRVRLRNDSSTPVEVHIKVGDEKRTVTVNPGEEVEVTFSANDPHKFNRPQFTIEWGGQRQHFQHH

SEQ ID NO: 218 - protein, 77 aa, synthetic construct
  REGION 1..77: note = Synthetic peptide
RPKVQLELHGNKMRVRLRNDSSTPVEVHIKVGDEKRTVTVNPGEEVEVTFSTTDPRELKNATIQLHQGDQTVEYRVD

SEQ ID NO: 219 - protein, 76 aa, synthetic construct
  REGION 1..76: note = Synthetic peptide
EVEIEVRTKNGKIEVRVTNRSDRPVEVRMEKGGQRETYTAPPGSTVRVEFSPSDDRQKRPTVEVTVNGRRYEVRVH

SEQ ID NO: 220 - protein, 71 aa, synthetic construct
  REGION 1..71: note = Synthetic peptide
RVEFRLREEGDRYRLEIRTDRPGTIEIEVNGRRERYTANPGTTITVEGTRGEEVEVTVEYDGKRERWRFRM

SEQ ID NO: 221 - protein, 74 aa, synthetic construct
  REGION 1..74: note = Synthetic peptide
RVRWTWRISGNTIEFRFENNSDRPARVEIEVDGQRREYTVNPGERLELHFQAGAREIRVEVEVGKEKYEVRIRF

SEQ ID NO: 222 - protein, 73 aa, synthetic construct
  REGION 1..73: note = Synthetic peptide
RVEVRIREEGDKYELRIRNRSDRPAEVRIEKGGKRETYTVNPGEELRIEFPPGAPPGRVEVQVGDKKYEYTVK

SEQ ID NO: 223 - protein, 74 aa, synthetic construct
  REGION 1..74: note = Synthetic peptide
VVEVRLEGERIRVRNNSDRPATVHVEKDGQRETYTVNPGEELEITSPDSSQNKGLRLRIHVEVNGQRFTFEFTM

SEQ ID NO: 224 - protein, 68 aa, synthetic construct
  REGION 1..68: note = Synthetic peptide
SIEVRVKGDRYEFRNNSDKPATLEVEKNGKREEYHMNPGESVEVRGEPGQDIRFEMVMEGTTYRYRLS

SEQ ID NO: 225 - protein, 68 aa, synthetic construct
  REGION 1..68: note = Synthetic peptide
SIEVRVKGDRYEFRNNSDKPATLEVEKNGKREEYHMNPGESVEVRGPPGQDIRFEMTMDGTTYRYRLS

SEQ ID NO: 226 - protein, 68 aa, synthetic construct
  REGION 1..68: note = Synthetic peptide
DLEVRRKDGKFEFRNNSDKPATLEVEKDGQREEYRMNPGETIEVQAPPGQDVRFTVEMPGREYRYKLD

SEQ ID NO: 227 - protein, 74 aa, synthetic construct
  REGION 1..74: note = Synthetic peptide
TFEVRVQWSGNTIRVTVENQSDRPATVRIEYGNTTYQRTINPGDRLTVEFTGGPGEVHVEVEINGKREERTFTK

SEQ ID NO: 228 - protein, 79 aa, synthetic construct
  REGION 1..79: note = Synthetic peptide
EVQMRVEISGDTIRVEVRNNSDRPGRVEFEVGGVRTSYTMNPGERIEVEVTVSTAEKQGIKVEVHVEAGDEKRTYEFQM

SEQ ID NO: 229 - protein, 71 aa, synthetic construct
  REGION 1..71: note = Synthetic peptide
RVEVRVQEKNGKVEIRVRSDGPVRVEVEVGGQRREYTGNPGEEVEIEVTADQPVRVEVKAGDKKFTYTVSE

SEQ ID NO: 230 - protein, 75 aa, synthetic construct
  REGION 1..75: note = Synthetic peptide
MFRVEVREKNGRVEVRVENRSDRPGTVEVEVGGVRLRFTVNPGEELEIRMDVPNGRRVEIEIVGKGVKYSYEYTV

SEQ ID NO: 231 - protein, 70 aa, synthetic construct
  REGION 1..70: note = Synthetic peptide
SWEVRVRWKNGRLEVEIRNNSSQPGKVRIEFDGKRHEVHLNPGESTKWRFENPGGEFHVEAGKEKYTYTV

SEQ ID NO: 232 - protein, 67 aa, synthetic construct
  REGION 1..67: note = Synthetic peptide
RVEVRQSGNTIEIRSEGPGRLELEYNGQREEYTLNPGTRYEYEGRPGEEVRVEVEMNGQRYTYEVRS

SEQ ID NO: 233 - protein, 73 aa, synthetic construct
  REGION 1..73: note = Synthetic peptide
RSEVHVRFEGERIEIQIHNGTDKPARVEMEVNGQRYEYHMPPNSKMEYRVPLRQEIRFEVEVGGQRFTYRYTS

SEQ ID NO: 234 - protein, 71 aa, synthetic construct
  REGION 1..71: note = Synthetic peptide
RVEVRVTYKGNRVEVRVRNNSDRPVRFRVVGPGAKYELKGNPGTEMRVEIRVPNAREIEVEVNGQRQRYQM

SEQ ID NO: 235 - protein, 70 aa, synthetic construct
  REGION 1..70: note = Synthetic peptide
RIEVRVDNGRVRVRNGTDRPCRVRVTAGGETREYTVNPGTELEVELSPEQQNNAEVEVECGNEKYRFQLG

SEQ ID NO: 236 - protein, 5 aa, synthetic construct
  REGION 1..5: note = Synthetic peptide
AGARE

SEQ ID NO: 237 - protein, 10 aa, synthetic construct
  REGION 1..10: note = Synthetic peptide
ANDPHKFNRP

SEQ ID NO: 238 - protein, 6 aa, synthetic construct
  REGION 1..6: note = Synthetic peptide
APPGQD

SEQ ID NO: 239 - protein, 9 aa, synthetic construct
  REGION 1..9: note = Synthetic peptide
VSPEELKNL

SEQ ID NO: 240 - protein, 9 aa, synthetic construct
  REGION 1..9: note = Synthetic peptide
VSTAEKQGI

SEQ ID NO: 241 - protein, 5 aa, synthetic construct
  REGION 1..5: note = Synthetic peptide
DSSTP

SEQ ID NO: 242 - protein, 5 aa, synthetic construct
  REGION 1..5: note = Synthetic peptide
VQPGA

SEQ ID NO: 243 - protein, 6 aa, synthetic construct
  REGION 1..6: note = Synthetic peptide
GEPGQD

SEQ ID NO: 244 - protein, 5 aa, synthetic construct
  REGION 1..5: note = Synthetic peptide
GGPGE

SEQ ID NO: 245 - protein, 6 aa, synthetic construct
  REGION 1..6: note = Synthetic peptide
GPPGQD

SEQ ID NO: 246 - protein, 5 aa, synthetic construct
  REGION 1..5: note = Synthetic peptide
GRPGE

SEQ ID NO: 247 - protein, 5 aa, synthetic construct
  REGION 1..5: note = Synthetic peptide
GTDKP

SEQ ID NO: 248 - protein, 5 aa, synthetic construct
  REGION 1..5: note = Synthetic peptide
GTDRP

SEQ ID NO: 249 - protein, 6 aa, synthetic construct
  REGION 1..6: note = Synthetic peptide
GVPPGG

SEQ ID NO: 250 - protein, 4 aa, synthetic construct
  REGION 1..4: note = Synthetic peptide
IRAR

SEQ ID NO: 251 - protein, 8 aa, synthetic construct
  REGION 1..8: note = Synthetic peptide
ISPEEAKN

SEQ ID NO: 252 - protein, 9 aa, synthetic construct
  REGION 1..9: note = Synthetic peptide
ISPEELKNA

SEQ ID NO: 253 - protein, 5 aa, synthetic construct
  REGION 1..5: note = Synthetic peptide
KSDRP

SEQ ID NO: 254 - protein, 5 aa, synthetic construct
  REGION 1..5: note = Synthetic peptide
KSSQP

SEQ ID NO: 255 - protein, 8 aa, synthetic construct
  REGION 1..8: note = Synthetic peptide
LSPEQQNN

SEQ ID NO: 256 - protein, 4 aa, synthetic construct
  REGION 1..4: note = Synthetic peptide
LTKP

SEQ ID NO: 257 - protein, 6 aa, synthetic construct
  REGION 1..6: note = Synthetic peptide
NDSSTP

SEQ ID NO: 258 - protein, 6 aa, synthetic construct
  REGION 1..6: note = Synthetic peptide
VPLRQE

SEQ ID NO: 259 - protein, 5 aa, synthetic construct
  REGION 1..5: note = Synthetic peptide
VPNGR

SEQ ID NO: 260 - protein, 6 aa, synthetic construct
  REGION 1..6: note = Synthetic peptide
NNSDRP

SEQ ID NO: 261 - protein, 10 aa, synthetic construct
  REGION 1..10: note = Synthetic peptide
TTDPRELKNA

SEQ ID NO: 262 - protein, 4 aa, synthetic construct
  REGION 1..4: note = Synthetic peptide
NPGD

SEQ ID NO: 263 - protein, 4 aa, synthetic construct
  REGION 1..4: note = Synthetic peptide
NPGE

SEQ ID NO: 264 - protein, 5 aa, synthetic construct
  REGION 1..5: note = Synthetic peptide
NPGEE

SEQ ID NO: 265 - protein, 4 aa, synthetic construct
  REGION 1..4: note = Synthetic peptide
NPGG

SEQ ID NO: 266 - protein, 4 aa, synthetic construct
  REGION 1..4: note = Synthetic peptide
NPGT

SEQ ID NO: 267 - protein, 5 aa, synthetic construct
  REGION 1..5: note = Synthetic peptide
NSDKP

SEQ ID NO: 268 - protein, 5 aa, synthetic construct
  REGION 1..5: note = Synthetic peptide
NSDQP

SEQ ID NO: 269 - protein, 5 aa, synthetic construct
  REGION 1..5: note = Synthetic peptide
NSDRP

SEQ ID NO: 270 - protein, 5 aa, synthetic construct
  REGION 1..5: note = Synthetic peptide
NSSQP

SEQ ID NO: 271 - protein, 5 aa, synthetic construct
  REGION 1..5: note = Synthetic peptide
PGKRP

SEQ ID NO: 272 - protein, 5 aa, synthetic construct
  REGION 1..5: note = Synthetic peptide
TSPKP

SEQ ID NO: 273 - protein, 6 aa, synthetic construct
  REGION 1..6: note = Synthetic peptide
PPGAPP

SEQ ID NO: 274 - protein, 4 aa, synthetic construct
  REGION 1..4: note = Synthetic peptide
PPGS

SEQ ID NO: 275 - protein, 4 aa, synthetic construct
  REGION 1..4: note = Synthetic peptide
PPGT

SEQ ID NO: 276 - protein, 5 aa, synthetic construct
  REGION 1..5: note = Synthetic peptide
PPNSK

SEQ ID NO: 277 - protein, 5 aa, synthetic construct
  REGION 1..5: note = Synthetic peptide
QSDRP

SEQ ID NO: 278 - protein, 5 aa, synthetic construct
  REGION 1..5: note = Synthetic peptide
RAPSP

SEQ ID NO: 279 - protein, 11 aa, synthetic construct
  REGION 1..11: note = Synthetic peptide
TSPDSSQNKGL

SEQ ID NO: 280 - protein, 5 aa, synthetic construct
  REGION 1..5: note = Synthetic peptide
RGKTP

SEQ ID NO: 281 - protein, 5 aa, synthetic construct
  REGION 1..5: note = Synthetic peptide
RSDQP

SEQ ID NO: 282 - protein, 5 aa, synthetic construct
  REGION 1..5: note = Synthetic peptide
RSDRP

SEQ ID NO: 283 - protein, 5 aa, synthetic construct
  REGION 1..5: note = Synthetic peptide
RSSQP

SEQ ID NO: 284 - protein, 6 aa, synthetic construct
  REGION 1..6: note = Synthetic peptide
RVPNAR

SEQ ID NO: 285 - protein, 10 aa, synthetic construct
  REGION 1..10: note = Synthetic peptide
SPSDDRQKRP

SEQ ID NO: 286 - protein, 5 aa, synthetic construct
  REGION 1..5: note = Synthetic peptide
TADQP

SEQ ID NO: 287 - protein, 4 aa, synthetic construct
  REGION 1..4: note = Synthetic peptide
APGT

SEQ ID NO: 288 - protein, 4 aa, synthetic construct
  REGION 1..4: note = Synthetic peptide
GKGV

SEQ ID NO: 289 - protein, 4 aa, synthetic construct
  REGION 1..4: note = Synthetic peptide
KGNR

SEQ ID NO: 290 - protein, 4 aa, synthetic construct
  REGION 1..4: note = Synthetic peptide
RGDS

SEQ ID NO: 291 - protein, 140 aa, synthetic construct
  REGION 1..140: note = Synthetic peptide
GRVEVRVEFEGDKMRVRLRNDSSTPVEVHIKVGDEKRTVTVNPGEEVEVTFSANDPHKFNRPQFTIEWGGGGRVEVRVEFEGDKMRVRLRNDSSTPVEVHIKVGDEKRTVTVNPGEEVEVTFSANDPHKFNRPQFTIEWG

SEQ ID NO: 292 - protein, 160 aa, synthetic construct
  REGION 1..160: note = Synthetic peptide
TETIEVRVDNGRVRVRNGTDRPIRVRVTAGGETREYTVNPGTELEVELSPEQQNQAIVVIHIGNEVFMFVLARDEEWVKRAEKLAEELNVRILVIVLNGRVRVRNGTDRPMRVRVTAGGETREYTVNPGTELEVELSPEQQNNAEVEVEVGNRKWRFQLG

SEQ ID NO: 293 - protein, 158 aa, synthetic construct
  REGION 1..158: note = Synthetic peptide
RIEVRVDNGRVRVRNGTDRPIRVRVTAGGETREYTVNPGTELEVELSPEQQNQAIVVVHIGNRVFMWVLARDEEWVKRAEKLAEELNVRILVIVLNGRVRVRNGTDRPMRVRVTAGGETREYTVNPGTELEVELSPEQQNNAEVEVEVGNEKWRFQLG

SEQ ID NO: 294 - protein, 159 aa, synthetic construct
  REGION 1..159: note = Synthetic peptide
TQTIEVRVDNGRVRVRNGTDRPIRVRVTAGGETREYTVNPGTELEVELSPEQQNNAMVEVQVGNEIVFFILAHNEELAKRWWEEAKQRAKILVMVLNGRVRVRNGTDRPMRVRVTAGGETREYTVNPGTELEVELSPEQQNNAEVEVEVGNNKYRFQLG

SEQ ID NO: 295 - protein, 152 aa, synthetic construct
  REGION 1..152: note = Synthetic peptide
GRVEVRVEFEGDKMRVRLRNDSSTPVEVHIKVGDEKRTVTVNPGEEVEVTFSANDPHKFNRPQFTIEWKDDKDGDGYISAAEKGRVEVRVEFEGDKMRVRLRNDSSTPVEVHIKVGDEKRTVTVNPGEEVEVTFSANDPHKFNRPQFTIEWG

SEQ ID NO: 296 - protein, 169 aa, synthetic construct
  REGION 1..169: note = Synthetic peptide
GRVEVRVEFEPIEDKDGDGYISAAEAAAAKMRVRLRNDSSTPVEVHIKVGDEKRTVTVNPGEEVEVTFSANDPHKFNRPQFTIEWKDDKDGDGYISAAEKGRVEVRVEFEGDKMRVRLRNDSSTPVEVHIKVGDEKRTVTVNPGEEVEVTFSANDPHKFNRPQFTIEWG

SEQ ID NO: 297 - protein, 172 aa, synthetic construct
  REGION 1..172: note = Synthetic peptide
GRVEVRVEFEGDKMRVRLRNDSSTPVEVHIKVGGDKDGDGYISAAEAKDAEKDAEKRTVTVNPGEEVEVTFSANDPHKFNRPQFTIEWKDDKDGDGYISAAEKGRVEVRVEFEGDKMRVRLRNDSSTPVEVHIKVGDEKRTVTVNPGEEVEVTFSANDPHKFNRPQFTIEWG

SEQ ID NO: 298 - protein, 174 aa, synthetic construct
  REGION 1..174: note = Synthetic peptide
GRVEVRVEFEPIEDKDGDGYISAAEAAAAKMRVRLRNDSSTPVEVHIKVGDEKRTVTVNPGEEVEVTFSANDPHKFNRPQFTIEWGGGGRVEVRVEFEPIEDKDGDGYISAAEAAAAKMRVRLRNDSSTPVEVHIKVGDEKRTVTVNPGEEVEVTFSANDPHKFNRPQFTIEWG

SEQ ID NO: 299 - protein, 169 aa, synthetic construct
  REGION 1..169: note = Synthetic peptide
GRVEVRVEFEGDKMRVRLRNDSSTPVEVHIKVGDEKRTVTVNPGEEVEVTFSANDPHKFNRPQFTIEWKDDKDGDGYISAAEKGRVEVRVEFEPIEDKDGDGYISAAEAAAAKMRVRLRNDSSTPVEVHIKVGDEKRTVTVNPGEEVEVTFSANDPHKFNRPQFTIEWG

SEQ ID NO: 300 - protein, 86 aa, synthetic construct
  REGION 1..86: note = Synthetic peptide
RIEVRVDNGRVRVRNGTDRPCRVRVTAGGETREYTVNPGTELEVELSPEQQNNAEVEVECTVDDKDGDGYISAAEAAVEKYRFQLG

SEQ ID NO: 301 - protein, 140 aa, synthetic construct; identical to SEQ ID NO: 291
  REGION 1..140: note = Synthetic peptide

SEQ ID NO: 302 - protein, 5 aa, synthetic construct
  REGION 1..5: note = Synthetic peptide
TQKRE

SEQ ID NO: 303 - protein, 12 aa, synthetic construct
  REGION 1..12: note = Synthetic peptide
DKDGDGYISAAE